SEQUENCE LISTING (15 sequences)

SEQ ID NO: 1 - RNA, 41 nt, Unknown
  Description of Unknown target coronavirus sequence
uaauuucuacuaaguguagaucccccagcgcuucagcguuc

SEQ ID NO: 2 - DNA, 10 bp, Artificial Sequence
  Description of Artificial Sequence Synthetic oligonucleotide
gtatgttaac

SEQ ID NO: 3 - DNA, 12 bp, Artificial Sequence
  Description of Artificial Sequence Synthetic oligonucleotide
cgcgttaacata

SEQ ID NO: 4 - DNA, 12 bp, Artificial Sequence
  Description of Artificial Sequence Synthetic oligonucleotide
caatagatcgcg

SEQ ID NO: 5 - DNA, 19 bp, Artificial Sequence
  Description of Artificial Sequence Synthetic oligonucleotide
tgggtagggcgggttggga

SEQ ID NO: 6 - DNA, 13 bp, Artificial Sequence
  Description of Artificial Sequence Synthetic oligonucleotide
  modified_base (8)..(8): Abasic nucleotide or mismatch base
tgggaggnatcta

SEQ ID NO: 7 - DNA, 22 bp, Artificial Sequence
  Description of Artificial Sequence Synthetic oligonucleotide
  modified_base (4)..(4): Abasic nucleotide or mismatch base
agcnctggtgggtagggcgggt

SEQ ID NO: 8 - DNA, 14 bp, Artificial Sequence
  Description of Artificial Sequence Synthetic oligonucleotide
tgggtagggcgggt

SEQ ID NO: 9 - DNA, 37 bp, Artificial Sequence
  Description of Artificial Sequence Synthetic oligonucleotide
gtaacgcgtagttgagctgtcacactgggggatctac

SEQ ID NO: 10 - DNA, 29 bp, Artificial Sequence
  Description of Artificial Sequence Synthetic oligonucleotide
ctgaagcgtgtgacgttgaagcgcgttac

SEQ ID NO: 11 - DNA, 24 bp, Artificial Sequence
  Description of Artificial Sequence Synthetic oligonucleotide
cgcgttaacatacaatagatcgcg

SEQ ID NO: 12 - DNA, 19 bp, Artificial Sequence
  Description of Artificial Sequence Synthetic oligonucleotide
tagatccctgtatgttaac

SEQ ID NO: 13 - DNA, 17 bp, Artificial Sequence
  Description of Artificial Sequence Synthetic oligonucleotide
gatctatttccagcgct

SEQ ID NO: 14 - DNA, 22 bp, Artificial Sequence
  Description of Artificial Sequence Synthetic oligonucleotide
tagatccctgggtagggcgggt

SEQ ID NO: 15 - DNA, 13 bp, Artificial Sequence
  Description of Artificial Sequence Synthetic oligonucleotide
tgggaccagcgct

(19) United States
(12) Patent Application Publication
(10) Pub. No.: US 2023/0212702 A1
(43) Pub. Date: Jul. 6, 2023

(54) MULTI-SENSING OF NUCLEIC ACID AND SMALL MOLECULE MARKERS

(71) Applicant: LogicInk Corporation, San Francisco, CA (US)

(72) Inventors: Hendrik DIETZ, Haar (DE); Carlos Edel OLGUIN ALVAREZ, San Francisco, CA (US)

(21) Appl. No.: 18/000,615

(22) PCT Filed: Jun. 4, 2021
(86) PCT No.: PCT/US2021/035993

§ 371 Date: Dec. 2, 2022

Related U.S. Application Data
(60) Provisional application No. 63/039,247, filed on Jun. 15, 2020.
(60) Provisional application No. 63/034,824, filed on Jun. 4, 2020.

(51) Int. Cl. C12Q 1/70; C12Q 1/6818

(52) U.S. Cl. CPC C12Q 1/701; C12Q 1/6818

(57) ABSTRACT

Systems and methods for determining the presence or absence of an analyte including a nucleic acid (e.g., DNA and RNA), a small molecule (e.g., proteins and amino acid chains), and one or more electrolytes (e.g., Na⁺ and K⁺). The system or method may detect multiple analytes (e.g., a first DNA and a second DNA) and/or multiple types of analytes (e.g., an RNA and an antibody protein). The signal readout provided by the system or method may be readily understood and may be correlated with a health condition (e.g., hydration or exposure to an infectious agent). The system may be wearable and may analyze one or more biofluids.

### CROSS-REFERENCE TO RELATED APPLICATIONS

This application claims priority under 35 U.S.C. § 119(e) to U.S. Provisional Application No. 63/034,824, entitled “Multi-Sensing of Markers from Body, Surfaces, and Environment,” filed Jun. 4, 2020, and U.S. Provisional Application No. 63/039,247, entitled “Multi-Sensing of Markers from Body, Surfaces, and Environment,” filed Jun. 15, 2020, each of which is incorporated by reference herein in its entirety.

### SEQUENCE LISTING

The instant application contains a Sequence Listing which has been submitted electronically in ASCII format and is hereby incorporated by reference in its entirety. Said ASCII copy, created on Jun. 4, 2021, is named 2212508_00136W02_SL.txt and is 3,976 bytes in size.

### BACKGROUND

### Field of the Invention

This application generally relates to systems and methods to detect nucleic acid and/or small molecule targets and/or provide a readily understandable readout indicating specific environmental or exposure conditions.

### Description of Related Art

There is a need for simple and readily understandable, low-cost, non-invasive, semi-continuous use, accumulated and/or real-time multi-sensing of markers from a broad set of sources including human bodies, animals, object surfaces, environments without requiring the use of additional electronic devices.

A person is exposed to a range of environments on a daily basis. The conditions of these environments and the length of exposure to these conditions may impact a person's mental and/or physical state. Several of these conditions may go undetected. Further, their impact on a person exposed to these conditions are not immediately apparent. For example, the microbiome present on a person's skin may be indicative of the individual's health and is not immediately apparent.

Human bodies area continuously exposed to microbial cells and their byproducts which can include toxic metabolites. Circulation of toxic metabolites may contribute to the onset of cancer. In addition, microbes may migrate throughout the human body and become associated with tumor development. Further, the presence or absence of specific microflora in a microbiome has been found to be associated with various health conditions including cancer, chronic inflammation, hydration levels, skin hydration levels, immune system disfunction, atopic dermatitis, psoriasis, acne vulgaris, skin ulcers, and conditions associated with aging. These microbiomes include those from a subject's gut, skin, and other topical areas of the body.

Additionally, a person commonly comes in contact with a myriad of infectious agents including microbial cells such as bacterial cells and virions. An immediately pressing example of an environmental condition that is not immediately apparent is the presence of viral components, such as those of the novel corona virus (e.g., SARS-CoV-2). Thus, it would be beneficial to identify and analyze the presence of infections agents such as viral components to determine exposure to such components.

Thus, it would be beneficial to identify and analyze the microbiome of a subject (or nucleic acids and/or small molecules thereof) and/or indicators of environmental exposures that may be deleterious (e.g., nucleic acids present in the SARS-CoV-2 virion or produced by the human body as a consequence of exposure to SARS-CoV-2) to be used to detect or predict via correlation the occurrence of carcinogenic conditions, inflammation disorders, potential infections, and other health conditions.

Despite the above needs, traditional electronic devices are bulky, battery-powered, expensive, and/or difficult to learn. Further, previous technology commonly relied on not readily available laboratory equipment such as gel electrophoretic equipment. Thus, there is a need for technology that is capable of identifying and analyzing the instant condition or exposure and highly sensitive, specific, low-cost, instrument-free, capable to work at body temperature, and/or wearable either for a day or more.

### BRIEF SUMMARY OF INVENTION

The present system or method disclosed herein may be directed towards a multi-sensing system and methods for detecting the presence of a target analyte such as a specific nucleic acid or a small molecule. The system or method further may include a method of signal amplification and/or a readily understandable readout.

In at least one aspect the invention is a system of detecting a nucleic acid target comprising a first target sequence and a second target sequence, wherein the system comprises:

a first nucleotide comprising a first nucleotide sequence configured to reversibly hybridize the first target sequence;
a second nucleotide comprising a second nucleotide sequence configured to reversibly hybridize the second target sequence;
wherein the first nucleotide and the second nucleotide are configured to dimerize to form a first dimer upon reversible hybridization of the first nucleotide sequence to the first target sequence and reversible hybridization of the second nucleotide sequence to the second target sequence;
a first reporter comprising a first reporter moiety and a first reporter sequence coupled to the first reporter moiety, wherein the first reporter sequence is configured to reversibly hybridize the first nucleotide sequence; and
a second reporter comprising a second reporter moiety and a second reporter sequence coupled to the second reporter moiety, wherein the second reporter sequence is configured to reversibly hybridize the second nucleotide sequence, and wherein the second reporter sequence is configured to reversibly hybridize the first reporter sequence;
wherein the first reporter and the second reporter are configured to dimerize to form a reporter dimer upon reversible hybridization of the first reporter sequence to the first nucleotide sequence of the first dimer and reversible hybridization of the second reporter sequence to the second nucleotide sequence of the first dimer,
wherein the first reporter moiety is configured to produce a first reporter moiety signal,
wherein the second reporter moiety is configured to alter the first reporter moiety signal when the first reporter moiety and the second reporter moiety are in proximity,
wherein reversible hybridization of the first reporter sequence to the second reporter sequence is configured to bring the first reporter moiety and the second reporter moiety into proximity, and
wherein reversible hybridization of one or more of the first reporter sequence to the first nucleotide sequence of the first dimer and the second reporter sequence to the second nucleotide sequence of the first dimer is configured to bring the first reporter moiety and the second reporter moiety out of proximity.

In at least one aspect the invention is a system of detecting a nucleic acid target comprising a first target sequence and a second target sequence, wherein the system comprises:

a first nucleotide comprising a first nucleotide sequence configured to reversibly hybridize the first target sequence;
a second nucleotide comprising a second nucleotide sequence configured to reversibly hybridize the second target sequence;
wherein the first nucleotide and the second nucleotide are configured to dimerize to form a first dimer upon reversible hybridization of the first nucleotide sequence to the first target sequence and reversible hybridization of the second nucleotide sequence to the second target sequence;
a first probe comprising a first probe sequence and a second probe sequence, wherein the first probe sequence is configured to reversibly hybridize the first nucleotide sequence;
a second probe comprising a third probe sequence and a fourth probe sequence, wherein the third probe sequence is configured to reversibly hybridize the second nucleotide sequence;
wherein the first probe and the second probe are configured to dimerize to form a first probe dimer upon reversible hybridization of the first probe sequence to the first nucleotide
sequence of the first dimer and reversible hybridization of the third probe sequence to the second nucleotide sequence of the first dimer; and
a reporter comprising:

a first reporter sequence configured to reversibly hybridize the second probe sequence,

a second reporter sequence coupled to the first reporter sequence, wherein the second reporter sequence is configured to reversibly hybridize the fourth probe sequence, and wherein the second reporter sequence is configured to reversibly hybridize the first reporter sequence,

a first reporter moiety coupled to first reporter sequence, and

a second reporter moiety coupled to the second reporter sequence;

wherein the first reporter moiety is configured to produce a first reporter moiety signal,
wherein the second reporter moiety is configured to alter the first reporter moiety signal when the first reporter moiety and the second reporter moiety are in proximity,
wherein reversible hybridization of the first reporter sequence to the second reporter sequence is configured to bring the first reporter moiety and the second reporter moiety into proximity, and
wherein reversible hybridization of one or more of the first reporter sequence to the second probe sequence of the first probe dimer and the second reporter sequence to the fourth probe sequence of the first probe dimer is configured to bring the first reporter moiety and the second reporter moiety out of proximity.

In at least one aspect the invention is a system of detecting a nucleic acid target comprising a first target sequence and a second target sequence, wherein the system comprises:

a first nucleotide comprising a first nucleotide sequence and a first enzymatic sequence coupled to the first nucleotide sequence, wherein the first nucleotide sequence is configured to reversibly hybridize the first target sequence;
a second nucleotide comprising a second nucleotide sequence and a second enzymatic sequence coupled to the second nucleotide sequence, wherein the second nucleotide sequence is configured to reversibly hybridize the second target sequence;
wherein the first nucleotide and the second nucleotide are configured to dimerize to form a first enzymatically active dimer upon reversible hybridization of the first nucleotide sequence to the first target sequence and reversible hybridization of the second nucleotide sequence to the second target sequence; and
one or more first substrates;
wherein the first enzymatically active dimer is configured to convert the one or more first substrates into one or more first products.

In at least one embodiment of any one of the aspects the system further comprises: a third nucleotide comprising a third nucleotide sequence and a third enzymatic sequence coupled to the third nucleotide sequence, wherein the third nucleotide sequence is configured to reversibly hybridize the first nucleotide sequence; and

a fourth nucleotide comprising a fourth nucleotide sequence and a fourth enzymatic sequence coupled to the fourth nucleotide sequence, wherein the fourth nucleotide sequence is configured to reversibly hybridize the second nucleotide sequence;
wherein the third nucleotide and the fourth nucleotide are configured to dimerize to form a second enzymatically active dimer upon reversible hybridization of the third nucleotide sequence to the first nucleotide sequence of the first enzymatically active dimer and the
fourth nucleotide sequence to the second nucleotide sequence of the first enzymatically active dimer, and
wherein the second enzymatically active dimer is configured to convert the one or more first substrates into one or more first products.

In at least one embodiment of any one of the aspects the system further comprises: a first seed nucleotide comprising a first seed sequence configured to reversibly hybridize the first nucleotide sequence; and

a second seed nucleotide comprising a second seed sequence configured to reversibly hybridize the second nucleotide sequence;
wherein the first seed nucleotide and the second seed nucleotide are configured to dimerize to form a first seed dimer upon reversible hybridization of the first seed nucleotide to the first nucleotide sequence of the first enzymatically active dimer and reversible hybridization of the second seed sequence to the second nucleotide sequence of the first enzymatically active dimer; and
wherein the first nucleotide and the second nucleotide are configured to dimerize to form the first enzymatically active dimer upon reversible hybridization of the first nucleotide sequence to the first seed sequence and reversible hybridization of the second nucleotide sequence to the second seed sequence.

In at least one embodiment of any one of the aspects one or more of the nucleic acid target, the first nucleotide, and the second nucleotide is a DNA molecule.

In at least one embodiment of any one of the aspects the first nucleotide is a DNA molecule, the second nucleotide is a DNA molecule, the first nucleotide comprises a first thymine base, the second nucleotide comprises a second thymine base, the first thymine base and the second thymine base are configured to be brought into proximity by the reversible hybridization of the first nucleotide sequence to the first target sequence and the reversible hybridization of the second nucleotide sequence to the second target sequence, and the first thymine base and the second thymine base are configured to dimerize when exposed to ultraviolet light.

In at least one embodiment of any one of the aspects one or more of the first probe and the second probe is a DNA molecule.

In at least one embodiment of any one of the aspects the first probe is a DNA molecule, the second probe is a DNA molecule, the first probe comprises a first thymine base, the second probe comprises a second thymine base, the first thymine base and the second thymine base are configured to be brought into proximity by the reversible hybridization of the first nucleotide sequence to the first probe sequence and the reversible hybridization of the second nucleotide sequence to the third probe sequence, and the first thymine base and the second thymine base are configured to dimerize when exposed to ultraviolet light.

In at least one embodiment of any one of the aspects one or more of the first reporter and the second reporter is a DNA molecule.

In at least one embodiment of any one of the aspects the first reporter is a DNA molecule, the second reporter is a DNA molecule, the first reporter comprises a first thymine base, the second reporter comprises a second thymine base, the first thymine base and the second thymine base are configured to be brought into proximity by the reversible hybridization of the first reporter sequence to the first nucleotide sequence and the reversible hybridization of the second reporter sequence to the second nucleotide sequence, and the first thymine base and the second thymine base are configured to dimerize when exposed to ultraviolet light.

In at least one embodiment of any one of the aspects the reporter is a DNA molecule.

In at least one embodiment of any one of the aspects one or more of the third nucleotide and the fourth nucleotide is a DNA molecule.

In at least one embodiment of any one of the aspects the third nucleotide is a DNA molecule, the fourth nucleotide is a DNA molecule, the third nucleotide comprises a first thymine base, the fourth nucleotide comprises a second thymine base, the first thymine base and the second thymine base are configured to be brought into proximity by the reversible hybridization of the third nucleotide sequence to the first nucleotide sequence and the reversible hybridization of the fourth nucleotide sequence to the second nucleotide sequence, and the first thymine base and the second thymine base are configured to dimerize when exposed to ultraviolet light.

In at least one embodiment of any one of the aspects one or more of the first seed nucleotide and the second seed nucleotide is a DNA molecule.

In at least one embodiment of any one of the aspects the first seed nucleotide is a DNA molecule, the second seed nucleotide is a DNA molecule, the first seed nucleotide comprises a first thymine base, the second seed nucleotide comprises a second thymine base, the first thymine base and the second thymine base are configured to be brought into proximity by the reversible hybridization of the first seed nucleotide sequence to the first nucleotide sequence and the reversible hybridization of the second seed nucleotide sequence to the second nucleotide sequence, and the first thymine base and the second thymine base are configured to dimerize when exposed to ultraviolet light.

In at least one embodiment of any one of the aspects one or more of the nucleic acid target, the first nucleotide, and the second nucleotide is an RNA molecule.

In at least one embodiment of any one of the aspects one or more of the first probe and the second probe is an RNA molecule.

In at least one embodiment of any one of the aspects one or more of the first reporter and the second reporter is an RNA molecule.

In at least one embodiment of any one of the aspects the reporter is an RNA molecule.

In at least one embodiment of any one of the aspects one or more of the third nucleotide and the fourth nucleotide is an RNA molecule.

In at least one embodiment of any one of the aspects one or more of the first seed nucleotide and the second seed nucleotide is an RNA molecule.

In at least one embodiment of any one of the aspects the first enzymatic sequence and the second enzymatic sequence are configured to form a deoxyribozyme or a ribozyme.

In at least one embodiment of any one of the aspects the third enzymatic sequence and the fourth enzymatic sequence are configured form a deoxyribozyme or a ribozyme.

In at least one embodiment of any one of the aspects one or more of the first nucleotide and the second nucleotide comprises one or more abasic sites configured to decrease the energy associated with dissociating the first nucleotide or the second nucleotide and a hybridization partner.

In at least one embodiment of any one of the aspects the first nucleotide sequence comprises one or more mismatch bases compared to the first target sequence configured to decrease the energy associated with dissociating the first nucleotide sequence and the first target sequence, and/or the second nucleotide sequence comprises one or more mismatch bases compared to the second target sequence configured to decrease the energy associated with dissociating the first nucleotide sequence and the first target sequence.

In at least one embodiment of any one of the aspects the first reporter sequence comprises one or more mismatch bases compared to the second reporter sequence configured to decrease the energy associated with dissociating the first reporter sequence and the second reporter sequence.

In at least one embodiment of any one of the aspects the first probe sequence comprises one or more mismatch bases compared to the first nucleotide sequence configured to decrease the energy associated with dissociating the first probe sequence and the first nucleotide sequence,

the second probe sequence comprises one or more mismatch bases compared to the first reporter sequence configured to decrease the energy associated with dissociating the second probe sequence and the first reporter sequence, the third probe sequence comprises one or more mismatch bases compared to the second nucleotide sequence configured to decrease the energy associated with dissociating the third probe sequence and the second nucleotide sequence, and/or
the fourth probe sequence comprises one or more mismatch bases compared to the second reporter sequence configured to decrease the energy associated with dissociating the fourth probe sequence and the second nucleotide sequence.

In at least one embodiment of any one of the aspects the first nucleotide sequence comprises one or more mismatch bases compared to the third nucleotide sequence configured to decrease the energy associated with dissociating the first nucleotide sequence and the third nucleotide sequence, and/or the second nucleotide sequence comprises one or more mismatch bases compared to the fourth nucleotide sequence configured to decrease the energy associated with dissociating the second nucleotide sequence and the fourth nucleotide sequence.

In at least one embodiment of any one of the aspects the first nucleotide sequence comprises one or more mismatch bases compared to the first seed sequence configured to decrease the energy associated with dissociating the first nucleotide sequence and the first seed sequence, and/or the second nucleotide sequence comprises one or more mismatch bases compared to the second seed sequence configured to decrease the energy associated with dissociating the second nucleotide sequence and the second seed sequence.

In at least one embodiment of any one of the aspects the first reporter moiety in proximity with the second reporter moiety is configured to increase fluorescence at a predetermined wavelength.

In at least one embodiment of any one of the aspects the first reporter moiety and the second reporter moiety are configured for Forster resonance energy transfer.

In at least one embodiment of any one of the aspects the first reporter moiety in proximity with the second reporter moiety is configured to decrease fluorescence at a predetermined wavelength.

In at least one embodiment of any one of the aspects the first reporter moiety is a fluorophore, and the second reporter moiety is a quencher.

In at least one embodiment of any one of the aspects the first enzymatic sequence and the second enzymatic sequence are configured to form a peroxidase-mimicking G-quadruplex deoxyribozyme.

In at least one embodiment of any one of the aspects the third enzymatic sequence and the fourth enzymatic sequence are configured to form a peroxidase-mimicking G-quadruplex deoxyribozyme.

In at least one embodiment of any one of the aspects the one or more first substrates comprises 2,2′-azino-bis(3-ethylbenzothiozoline-6-sulfonic acid) (ABTS) or 3,3′,5,5′-tetramethylbenzidine (TMB), the system further comprises hydrogen peroxide (H₂O₂), and the system further comprises hemin.

In at least one embodiment of any one of the aspects a second target comprises a third target sequence and a fourth target sequence, and wherein the system further comprises: a fifth nucleotide comprising a fifth nucleotide sequence configured to reversibly hybridize the third target sequence;

a sixth nucleotide comprising a sixth nucleotide sequence configured to reversibly hybridize the fourth target sequence;
wherein the fifth nucleotide and the sixth nucleotide are configured to dimerize to form a second dimer upon reversible hybridization of the fifth nucleotide sequence to the third target sequence and reversible hybridization of the sixth nucleotide sequence to the fourth target sequence;
a third reporter comprising a third reporter moiety and a third reporter sequence coupled to the third reporter moiety, wherein the third reporter sequence is configured to reversibly hybridize the fifth nucleotide sequence; and
a fourth reporter comprising a fourth reporter moiety and a fourth reporter sequence coupled to the fourth reporter moiety, wherein the fourth reporter sequence is configured to reversibly hybridize the sixth nucleotide sequence, and wherein the fourth reporter sequence is configured to reversibly hybridize the third reporter sequence;
wherein the third reporter and the fourth reporter are configured to dimerize to form a second reporter dimer upon reversible hybridization of the third reporter sequence to the fifth nucleotide sequence of the second dimer and reversible hybridization of the fourth reporter sequence to the sixth nucleotide sequence of the second dimer,
wherein the third reporter moiety is configured to produce a second reporter moiety signal, wherein the fourth reporter moiety is configured to alter the second reporter moiety signal when the third reporter moiety and the fourth reporter moiety are in proximity,
wherein reversible hybridization of the third reporter sequence to the fourth reporter sequence is configured to bring the third reporter moiety and the fourth reporter moiety into proximity, and
wherein reversible hybridization of one or more of the third reporter sequence to the fifth nucleotide sequence of the second dimer and the fourth reporter sequence to the sixth nucleotide sequence of the second dimer is configured to bring the third reporter moiety and the fourth reporter moiety out of proximity.

In at least one embodiment of any one of the aspects a second target comprises a third target sequence and a fourth target sequence, and wherein the system further comprises:

a fifth nucleotide comprising a fifth nucleotide sequence configured to reversibly hybridize the third target sequence;
a sixth nucleotide comprising a sixth nucleotide sequence configured to reversibly hybridize the fourth target sequence;
wherein the fifth nucleotide and the sixth nucleotide are configured to dimerize to form a second dimer upon reversible hybridization of the fifth nucleotide sequence to the third target sequence and reversible hybridization of the sixth nucleotide sequence to the fourth target sequence;
a third probe comprising a fifth probe sequence and a sixth probe sequence, wherein the fifth probe sequence is configured to reversibly hybridize the fifth nucleotide sequence;
a fourth probe comprising a seventh probe sequence and an eighth probe sequence, wherein the seventh probe sequence is configured to reversibly hybridize the sixth nucleotide sequence;
wherein the third probe and the fourth probe are configured to dimerize to form a second probe dimer upon reversible hybridization of the fifth probe sequence to the fifth nucleotide sequence of the second dimer and reversible hybridization of the seventh probe sequence to the sixth nucleotide sequence of the second dimer; and
a second reporter comprising:

a third reporter sequence configured to reversibly hybridize the sixth probe sequence,

a fourth reporter sequence coupled to the third reporter sequence, wherein the fourth reporter sequence is configured to reversibly hybridize the eighth probe sequence, and wherein the fourth reporter sequence is configured to reversibly hybridize the third reporter sequence,

a third reporter moiety coupled to third reporter sequence, and

a fourth reporter moiety coupled to the fourth reporter sequence;

wherein the third reporter moiety is configured to produce a second reporter moiety signal, wherein the fourth reporter moiety is configured to alter the second reporter moiety signal when the third reporter moiety and the fourth reporter moiety are in proximity,
wherein reversible hybridization of the third reporter sequence to the fourth reporter sequence is configured to bring the third reporter moiety and fourth reporter moiety into proximity, and wherein reversible hybridization of one or more of the third reporter sequence to the sixth probe sequence of the second probe dimer and the fourth reporter sequence to the eighth probe sequence of the second probe dimer is configured to bring the third reporter moiety and the fourth reporter moiety out of proximity.

In at least one embodiment of any one of the aspects a second target comprises a third target sequence and a fourth target sequence, and wherein the system further comprises: a fifth nucleotide comprising a fifth nucleotide sequence and a third enzymatic sequence coupled to the fifth nucleotide sequence, wherein the fifth nucleotide sequence is configured to reversibly hybridize the third target sequence;

a sixth nucleotide comprising a sixth nucleotide sequence and a fourth enzymatic sequence coupled to the sixth nucleotide sequence, wherein the sixth nucleotide sequence is configured to reversibly hybridize the fourth target sequence;
wherein the fifth nucleotide and the sixth nucleotide are configured to dimerize to form a third enzymatically active dimer upon reversible hybridization of the fifth nucleotide sequence to the third target sequence and reversible hybridization of the sixth nucleotide sequence to the fourth target sequence; and
one or more second substrates;
wherein the third enzymatically active dimer is configured to convert the one or more second substrates into one or more second products, and
wherein the one or more second products are different from the one or more first products.

In at least one embodiment of any one of the aspects the system further comprises: a seventh nucleotide comprising a seventh nucleotide sequence and a fifth enzymatic sequence coupled to the seventh nucleotide sequence, wherein the seventh nucleotide sequence is configured to reversibly hybridize the fifth nucleotide sequence; and an eighth nucleotide comprising an eighth nucleotide sequence and a sixth enzymatic sequence coupled to the eighth nucleotide sequence, wherein the eighth nucleotide sequence is configured to reversibly hybridize the sixth nucleotide sequence;

wherein the seventh nucleotide and the eighth nucleotide are configured to dimerize to form a fourth enzymatically active dimer upon reversible hybridization of the seventh nucleotide sequence to the fifth nucleotide sequence of the third enzymatically active dimer and the eighth nucleotide sequence to the sixth nucleotide sequence of the third enzymatically active dimer, and
wherein the fourth enzymatically active dimer is configured to convert the one or more second substrates into one or more second products.

In at least one embodiment of any one of the aspects the system further comprises: a third seed nucleotide comprising a third seed sequence configured to reversibly hybridize the fifth nucleotide sequence; and

a fourth seed nucleotide comprising a fourth seed sequence configured to reversibly hybridize the sixth nucleotide sequence;
wherein the third seed nucleotide and the fourth seed nucleotide are configured to dimerize to form a second seed dimer upon reversible hybridization of the third seed nucleotide to the fifth nucleotide sequence of the third enzymatically active dimer and reversible hybridization of the fourth seed sequence to the sixth nucleotide sequence of the third enzymatically active dimer; and
wherein the fifth nucleotide and the sixth nucleotide are configured to dimerize to form the third enzymatically active dimer upon reversible hybridization of the fifth nucleotide sequence to the third seed sequence and reversible hybridization of the sixth nucleotide sequence to the fourth seed sequence.

In at least one embodiment of any one of the aspects the one or more first substrates is 2,2′-azino-bis(3-ethylbenzothiozoline-6-sulfonic acid) (ABTS), the one or more first products is an ABTS radical, the one or more second substrates is 3,3′,5,5′-tetramethylbenzidine (TMB), and the one or more second products is a TMB radical.

In at least one embodiment of any one of the aspects the nucleic acid target comprises the second target.

In at least one embodiment of any one of the aspects the system further comprises a module comprising:

a sensor configured to detect ultraviolet light;
a pathway configured to output a comparison comparing the cumulative ultraviolet light detected by the sensor to a predetermined threshold; and
a display configured to display a value associated with one or more of the ultraviolet light detected by the sensor, the cumulative ultraviolet light detected by the sensor, and the comparison.

In at least one embodiment of any one of the aspects the system further comprises an ultraviolet light source.

In at least one aspect the invention is a method of detecting a nucleic acid target comprising a first target sequence and a second target sequence, wherein the method comprises:

providing a first nucleotide comprising a first nucleotide sequence;
reversibly hybridizing the first nucleotide sequence to the first target sequence;
providing a second nucleotide comprising a second nucleotide sequence;
reversibly hybridizing the second nucleotide sequence to the second target sequence;
dimerizing the first nucleotide and the second nucleotide to form a first dimer upon reversibly hybridizing the first nucleotide sequence to the first target sequence and reversibly hybridizing the second nucleotide sequence to the second target sequence;
dissociating the first dimer and the nucleic acid target;
providing a reporter complex comprising:
a first reporter comprising a first reporter moiety and a first reporter sequence coupled to the first reporter moiety, wherein the first reporter moiety is configured to produce a first reporter moiety signal, and
a second reporter comprising a second reporter moiety and a second reporter sequence coupled to the second reporter moiety, wherein the second reporter sequence is reversibly hybridized to the first reporter sequence, wherein the second reporter moiety is configured to alter the first reporter moiety signal when the first reporter moiety and the second reporter moiety are in proximity, and wherein reversible hybridization of the first reporter sequence to the second reporter sequence is configured to bring the first reporter moiety and the second reporter moiety into proximity;

dissociating the first reporter sequence and the second reporter sequence;

reversibly hybridizing the first reporter sequence to the first nucleotide sequence;

reversibly hybridizing the second reporter sequence to the second nucleotide sequence; bringing the first reporter moiety and the second reporter moiety out of proximity by reversibly hybridizing the first reporter sequence to the first nucleotide sequence of the first dimer and/or reversibly hybridizing the second reporter sequence to the second nucleotide sequence of the first dimer, and
detecting a change in the first reporter moiety signal.

In at least one embodiment of any one of the aspects the method further comprises dimerizing the first reporter and the second reporter to form a reporter dimer upon reversibly hybridizing the first reporter sequence to the first nucleotide sequence of the first dimer and reversibly hybridizing the second reporter sequence to the second nucleotide sequence of the first dimer.

In at least one embodiment of any one of the aspects the method further comprises dissociating the first dimer and the first reporter dimer.

In at least one aspect the invention is a method of detecting a nucleic acid target comprising a first target sequence and a second target sequence, wherein the method comprises:

providing a first nucleotide comprising a first nucleotide sequence;
reversibly hybridizing the first nucleotide sequence to the first target sequence; providing a second nucleotide comprising a second nucleotide sequence;
reversibly hybridizing the second nucleotide sequence to the second target sequence;
dimerizing the first nucleotide and the second nucleotide to form a first dimer upon reversibly hybridizing the first nucleotide sequence to the first target sequence and reversibly hybridizing the second nucleotide sequence to the second target sequence;
dissociating the first dimer and the nucleic acid target;
providing a first probe comprising a first probe sequence and a second probe sequence;
reversibly hybridizing the first probe sequence to the first nucleotide sequence;
providing a second probe comprising a third probe sequence and a fourth probe sequence;
reversibly hybridizing the third probe sequence to the second nucleotide sequence; provide a reporter comprising:

a first reporter moiety configured to produce a first reporter moiety signal,

a first reporter sequence coupled to the first reporter moiety, wherein the first reporter sequence is configured to reversibly hybridize the second probe sequence,

a second reporter moiety configured to alter the first reporter moiety signal when the first reporter moiety and the second reporter moiety are in proximity, and

a second reporter sequence, wherein the second reporter sequence is coupled to the second reporter moiety, wherein the second reporter sequence is coupled to the first reporter sequence, wherein the second reporter sequence is reversibly hybridized to the first reporter sequence, wherein the second reporter sequence is configured to reversibly hybridize the second nucleotide sequence, and wherein reversible hybridization of the first reporter sequence to the second reporter sequence is configured to bring the first reporter moiety and the second reporter moiety into proximity; and

dissociating the first reporter sequence and the second reporter sequence;
reversibly hybridizing the first reporter sequence to the second probe sequence;
reversibly hybridizing the second reporter sequence to the fourth probe sequence; bringing the first reporter moiety and the second reporter moiety out of proximity by reversible hybridizing the first reporter sequence to the second probe sequence and/or reversible hybridizing the second reporter sequence to the fourth probe sequence, and detecting a change in the first reporter moiety signal.

In at least one embodiment of any one of the aspects the method further comprises dimerizing the first probe and the second probe to form a first probe dimer upon reversible hybridizing the first probe sequence to the first nucleotide sequence of the first dimer and reversibly hybridizing the third probe sequence to the second nucleotide sequence of the first dimer.

In at least one aspect the invention is a method of detecting a nucleic acid target comprising a first target sequence and a second target sequence, wherein the method comprises:

providing a first nucleotide comprising a first nucleotide sequence and a first enzymatic sequence coupled to the first nucleotide sequence;
reversibly hybridizing the first nucleotide sequence to the first target sequence; providing a second nucleotide comprising a second nucleotide sequence and a second enzymatic sequence coupled to the second nucleotide sequence;
reversibly hybridizing the second nucleotide sequence to the second target sequence;
dimerizing the first nucleotide and the second nucleotide to form a first enzymatically active dimer upon reversibly hybridizing the first nucleotide sequence to the first target sequence and reversibly hybridizing the second nucleotide sequence to the second target sequence, wherein the first enzymatically active dimer is configured to convert one or more first substrates into one or more first products;
providing the one or more first substrates;
converting the one or more first substrates to the one or more first products; and detecting one or more of a decrease in amount of the one or more first substrates, a decrease in the concentration of the one or more first substrates, an increase in amount of the one or more first products, and an increase in concentration of the one or more first products.

In at least one embodiment of any one of the aspects the method further comprises: dissociating the first enzymatically active dimer and the nucleic acid target;

providing a first seed nucleotide comprising a first seed sequence;
reversibly hybridizing the first seed sequence the first nucleotide sequence;
providing a second seed nucleotide comprising a second seed sequence;
reversibly hybridizing the second seed nucleotide to the second nucleotide sequence; and dimerizing the first seed nucleotide and the second seed nucleotide to form a first seed dimer upon reversibly hybridizing the first seed nucleotide to the first nucleotide sequence of the first enzymatically active dimer and reversibly hybridizing the second seed sequence to the second nucleotide sequence of the first enzymatically active dimer.

In at least one embodiment of any one of the aspects the method further comprises: dissociating the first enzymatically active dimer and the nucleic acid target;

providing a third nucleotide comprising a third nucleotide sequence and a third enzymatic sequence coupled to the third nucleotide sequence;
reversibly hybridizing the third nucleotide sequence to the first nucleotide sequence; providing a fourth nucleotide comprising a fourth nucleotide sequence and a fourth enzymatic sequence coupled to the fourth nucleotide sequence;
reversibly hybridizing the fourth nucleotide sequence to the second nucleotide sequence; and dimerizing the third nucleotide and the fourth nucleotide to form a second enzymatically active dimer upon reversibly hybridizing the third nucleotide sequence to the first nucleotide sequence of the first enzymatically active dimer and reversibly hybridizing the fourth nucleotide sequence to the second nucleotide sequence of the first enzymatically active dimer, wherein the second enzymatically active dimer is configured to convert the one or more first substrates into the one or more first products.

In at least one embodiment of any one of the aspects first nucleotide comprises a first nucleotide thymine base; the second nucleotide comprises a second nucleotide thymine base; and the step of dimerizing the first nucleotide and the second nucleotide comprises: bringing the first nucleotide thymine base into proximity with the second nucleotide thymine base, providing an ultraviolet light source, and forming one or more bonds coupling the first nucleotide thymine base and the second nucleotide thymine base.

In at least one embodiment of any one of the aspects the first reporter comprises a first reporter thymine base; the second reporter comprises a second reporter thymine base; and the step of dimerizing the first reporter and the second reporter comprises: bringing the first reporter thymine base into proximity with the second reporter thymine base, providing an ultraviolet light source, and forming one or more bonds coupling the first reporter thymine base and the second reporter thymine base.

In at least one embodiment of any one of the aspects the third nucleotide comprises a third nucleotide thymine base; the fourth nucleotide comprises a fourth nucleotide thymine base; and the step of dimerizing the third nucleotide and the fourth nucleotide comprises: bringing the third nucleotide thymine base into proximity with the fourth nucleotide thymine base, providing an ultraviolet light source, and forming one or more bonds coupling the third nucleotide thymine base and the fourth nucleotide thymine base.

In at least one embodiment of any one of the aspects the first seed nucleotide comprises a first seed thymine base; the second seed nucleotide comprises a second seed nucleotide thymine base; and the step of dimerizing the first seed nucleotide and the second seed nucleotide comprises: bringing the first seed nucleotide thymine base into proximity with the second seed nucleotide thymine base, providing an ultraviolet light source, and forming one or more bonds coupling the first seed nucleotide thymine base and the second seed nucleotide thymine base.

In at least one embodiment of any one of the aspects the step of detecting the change in a signal produced by one or more of the first reporter moiety and the second reporter moiety comprises detecting one or more of an increase in fluorescence at a first predetermined wavelength and a decrease in fluorescence at a second predetermined wavelength.

In at least one embodiment of any one of the aspects the increase in fluorescence at the first predetermined wavelength is due to Forster resonance energy transfer, or the decrease in fluorescence at the second wavelength is due to Forster resonance energy transfer.

In at least one embodiment of any one of the aspects the first reporter moiety is a fluorophore, the second reporter moiety is a quencher, and the decrease in fluorescence at the second predetermined wavelength is due to quenching a fluorescent signal emitted by the first reporter moiety.

In at least one embodiment of any one of the aspects the first reporter moiety is a fluorophore, the second reporter moiety is a quencher, and the increase in fluorescence at the first predetermined wavelength is due to de-quenching a fluorescent signal emitted by the first reporter moiety.

In at least one embodiment of any one of the aspects the step of detecting one or more of the decrease in amount of the one or more first substrates, the decrease in the concentration of the one or more first substrates, the increase in amount of one or more first products, and the increase in concentration of one or more first products comprises detecting the increase in the amount of a 2,2′-azino-bis(3-ethylbenzothiozoline-6-sulfonic acid) (ABTS) radical, detecting the increase in the concentration of an ABTS radical, detecting the increase in the amount of a 3,3′,5,5′-tetramethylbenzidine (TMB) radical, or detecting the increase in the concentration of a TMB radical.

In at least one embodiment of any one of the aspects a second target comprises a third target sequence and a fourth target sequence, the method further comprises:

providing a fifth nucleotide comprising a fifth nucleotide sequence;
reversibly hybridizing the fifth nucleotide sequence to the third target sequence;
providing a sixth nucleotide comprising a sixth nucleotide sequence;
reversibly hybridizing the sixth nucleotide sequence to the fourth target sequence;
dimerizing the fifth nucleotide and the sixth nucleotide to form a second dimer, upon reversibly hybridizing the fifth nucleotide sequence to the third target sequence and reversibly hybridizing the sixth nucleotide sequence to the fourth target sequence;
dissociating the second dimer and the target;
providing a second reporter complex comprising:
a third reporter comprising a third reporter moiety and a third reporter sequence coupled to the third reporter moiety, and wherein the third reporter moiety is configured to produce a second reporter moiety signal, and
a fourth reporter comprising a fourth reporter moiety and a fourth reporter sequence coupled to the fourth reporter moiety, wherein the fourth reporter sequence is reversibly hybridized to the third reporter sequence, wherein the fourth reporter moiety is configured to alter the second reporter moiety signal when the third reporter moiety and the fourth reporter moiety are in proximity, and wherein reversible hybridization of the third reporter sequence to the fourth reporter sequence is configured to bring the third reporter moiety and the fourth reporter moiety into proximity;

dissociating the third reporter sequence and the fourth reporter sequence;

reversibly hybridizing the third reporter sequence to the fifth nucleotide sequence;

reversibly hybridizing the fourth reporter sequence to the sixth nucleotide sequence;

bringing the third reporter moiety and the fourth reporter moiety out of proximity by reversibly hybridizing the third reporter sequence to the fifth nucleotide sequence of the second dimer and/or reversibly hybridizing the fourth reporter sequence to the sixth nucleotide sequence of the second dimer, and

detecting a change in the second reporter moiety signal.

In at least one embodiment of any one of the aspects the method further comprises dimerizing the third reporter and the fourth reporter to form a second reporter dimer upon reversibly hybridizing the third reporter sequence to the fifth nucleotide sequence and reversibly hybridizing the fourth reporter sequence to the sixth nucleotide sequence.

In at least one embodiment of any one of the aspects the method further comprises dissociating the second dimer and the second reporter dimer.

In at least one embodiment of any one of the aspects a second target comprises a third target sequence and a fourth target sequence, the method further comprises:

providing a fifth nucleotide comprising a fifth nucleotide sequence;
reversibly hybridizing the fifth nucleotide sequence to the third target sequence;
providing a sixth nucleotide comprising a sixth nucleotide sequence;
reversibly hybridizing the sixth nucleotide sequence to the fourth target sequence;
dimerizing the fifth nucleotide and sixth nucleotide to form a second dimer upon reversibly hybridizing the fifth nucleotide sequence to the third target sequence and reversibly hybridizing the sixth nucleotide sequence to the fourth target sequence;
dissociating the second dimer and the second target;
providing a third probe comprising a fifth probe sequence and a sixth probe sequence;
reversibly hybridizing the fifth probe sequence to the fifth nucleotide sequence;
providing a fourth probe comprising a seventh probe sequence and an eighth probe sequence;
reversibly hybridizing the seventh probe sequence to the sixth nucleotide sequence;
providing a second reporter comprising:

a third reporter moiety configured to produce a second reporter moiety signal, a third reporter sequence coupled to the third reporter moiety, wherein the third reporter sequence is configured to reversibly hybridize the sixth probe sequence,

a fourth reporter moiety configured to alter the second reporter moiety signal when the third reporter moiety and the fourth reporter moiety are in proximity, and a fourth reporter sequence, wherein the fourth reporter sequence is coupled to the fourth reporter moiety, wherein the fourth reporter sequence is coupled to the third reporter sequence, wherein the fourth reporter sequence is reversibly hybridized to the third reporter sequence, wherein the fourth reporter sequence is configured to reversibly hybridize the eighth probe sequence, and wherein reversible hybridization of the third reporter sequence to the fourth reporter sequence is configured to bring the third reporter moiety and the fourth reporter moiety into proximity, and

dissociating the third reporter sequence and the fourth reporter sequence;
reversibly hybridizing the third reporter sequence to the sixth probe sequence;
reversibly hybridizing the fourth reporter sequence to the eighth probe sequence;
bringing the third reporter moiety and the fourth reporter moiety out of proximity by reversibly hybridizing the third reporter sequence to the sixth probe sequence and/or reversibly hybridizing the fourth reporter sequence to the eighth probe sequence, and

detecting a change in the second reporter moiety signal.

In at least one embodiment of any one of the aspects the method further comprises dimerizing the third probe and the fourth probe to form a second probe dimer upon reversibly hybridizing the fifth probe sequence to the fifth nucleotide sequence and reversibly hybridizing the seventh probe sequence to the sixth nucleotide sequence.

In at least one embodiment of any one of the aspects a second target comprises a third target sequence and a fourth target sequence, the method further comprises:

providing a fifth nucleotide comprising a fifth nucleotide sequence and a third enzymatic sequence coupled to the fifth nucleotide sequence;
reversibly hybridizing the fifth nucleotide sequence to the third target sequence; providing a sixth nucleotide comprising a sixth nucleotide sequence and a fourth enzymatic sequence coupled to the sixth nucleotide sequence;
reversibly hybridizing the sixth nucleotide sequence to the fourth target sequence;
dimerizing the fifth nucleotide and the sixth nucleotide to form a third enzymatically active dimer upon reversibly hybridizing the fifth nucleotide sequence to the third target sequence and reversibly hybridizing the sixth nucleotide sequence to the fourth target sequence, wherein the third enzymatically active dimer is configured to convert one or more second substrates into one or more second products, wherein the one or more second products are different from the one or more first products;
providing the one or more second substrates;
converting the one or more second substrates into the one or more second products; and
detecting one or more of a decrease in amount of the one or more second substrates, a decrease in the concentration of the one or more second substrates, an increase in amount of the one or more second products, and an increase in concentration of the one or more second products.

In at least one embodiment of any one of the aspects the method further comprises: dissociating the third enzymatically active dimer and the second target;

providing a third seed nucleotide comprising a third seed sequence;
reversibly hybridizing the third seed sequence to the fifth nucleotide sequence;
providing a fourth seed nucleotide comprising a fourth seed sequence;
reversibly hybridizing the fourth seed sequence to the sixth nucleotide sequence; and
dimerizing the third seed nucleotide and the fourth seed nucleotide to form a second seed dimer upon reversibly hybridizing the third seed sequence to the fifth nucleotide sequence of the third enzymatically active dimer and reversibly hybridizing the fourth seed sequence to the sixth nucleotide of the third enzymatically active dimer.

In at least one embodiment of any one of the aspects the method further comprises: dissociating the third enzymatically active dimer and the second target;

providing a seventh nucleotide comprising a seventh nucleotide sequence and a fifth enzymatic sequence coupled to the seventh nucleotide sequence;
reversibly hybridizing the seventh nucleotide sequence to the fifth nucleotide sequence; providing an eighth nucleotide comprising an eighth nucleotide sequence and a sixth enzymatic sequence coupled to the eighth nucleotide sequence;
reversibly hybridizing the eighth nucleotide sequence to the sixth nucleotide sequence; and dimerizing the seventh nucleotide and the eighth nucleotide to form a fourth enzymatically active dimer upon reversibly hybridizing the seventh nucleotide sequence to the fifth nucleotide sequence of the third enzymatically active dimer and reversibly hybridizing the eighth nucleotide sequence to the sixth nucleotide sequence of the third enzymatically active dimer, wherein the fourth enzymatically active dimer is configured to convert the one or more second substrates into the one or more second products.

In at least one embodiment of any one of the aspects the one or more first substrates is 2,2′-azino-bis(3-ethylbenzothiozoline-6-sulfonic acid) (ABTS), the one or more first products is an ABTS radical, the one or more second substrates is 3,3′,5,5′-tetramethylbenzidine (TMB), and the one or more second products is a TMB radical.

In at least one embodiment of any one of the aspects the nucleic acid target comprises the second target.

In at least one embodiment of any one of the aspects the method further comprising determining one or more of the concentrations of one or more cations, anions, and salts and the amount of one or more cations, anions, and salts of a composition comprising the nucleic acid target.

### BRIEF DESCRIPTION OF THE DRAWINGS

FIG. 1 depicts an exemplary embodiment of detecting the presence of a target nucleic acid.

FIG. 2 depicts an exemplary embodiment of detecting the presence of nucleic acids from the novel coronavirus, SARS-CoV2. FIG. 2 discloses SEQ ID NOS 1, 9, and 10, respectively, in order of appearance.

FIGS. 3A-3B depict embodiments for detecting a nucleic acid target. FIG. 3A depicts an embodiment for detecting nucleic acids from SARS-CoV-2. FIG. 3A discloses SEQ ID NOS 11-13 and 1 in order of appearance. FIG. 3B depicts another design to detect a nucleic acid.

FIG. 4 depicts an embodiment to detect a nucleic acid.

FIG. 5 depicts an embodiment to for detecting two nucleic acid targets.

FIG. 6 depicts an exemplary visual indicator.

FIG. 7 depicts an embodiment for detecting a target nucleic acid involving a Deoxyribozyme (DNAzyme).

FIG. 8 depicts an example of a Deoxyribozyme (DNAzyme) embodiment for detecting an RNA associated with SARS-CoV-2. FIG. 8 discloses SEQ ID NOS 14, 15, 1, 6, and 7, in order of appearance.

FIG. 9 depicts exemplary embodiments for detecting a nucleic acid target involving a Deoxyribozyme (DNAzyme). FIG. 9 discloses SEQ ID NOS 1, 6, 7, 14, and 15 in order of appearance.

FIG. 10 depicts an exemplary embodiment for detecting ionic strength of a solution.

FIG. 11 depicts a reaction network for an exemplary Deoxyribozyme (DNAzyme) embodiment.

### DETAILED DESCRIPTION

In one aspect, the present system or method is directed towards systems and/or methods for detecting one or more analytes using multimerization (e.g., dimerization) to provide one or more detectable signals. In some embodiments the detectable signal is amplified. In some embodiments there is a many-to-one correspondence between each molecule contributing to the detectable signal and each molecule of the analyte. In some embodiments, each molecule of the analyte corresponds to multiple molecules contributing to the detectable signal, e.g., each molecule of the analyte may correspond to two, tens, hundreds, thousands, or tens of thousands of molecules contributing to the detectable signal.

An analyte can be any detectable molecule of interest including but not limited to a nucleic acid and a small molecule. In some embodiments, the analyte is a nucleic acid, for example a DNA or an RNA. An analyte may be obtained from any appropriate source including, but not limited to, saliva, exhalation, sweat, the skin microbiome, or an object's surface. An analyte may be extracted by any suitable method known in the art. For example, extraction of a nucleic acid may be achieved by including a lysis buffer such as 10% protease K, 0.7 M NaCl, 0.1% Hexadecyl trimethyl ammonium Bromide (CTAB) and IVIES at pH 5.0. Other methods known in the art suitable for nucleic acids are contemplated.

In some embodiments, the analyte may undergo a pre-detection amplification step such as whole genome amplification. This amplification increases the amount of a genome (e.g., a viral genome) available that may possess the analyte of interest. Such whole genome amplification may increase the likelihood that there is sufficient analyte available to generate detectable signal without the use of an additional instrument (e.g., by the naked eye). Exemplary whole genome amplification systems may be based on Phi29 or any known polymerase. In some embodiments, the polymerase is isothermal and is enzymatically active at skin temperature (e.g., Phi29).

In another aspect, the generated signal undergoes an exponential amplification for subsequent detection. In some embodiments, no analyte amplification step (e.g., whole genome amplification) may be needed. In some embodiments, an analyte amplification step may precede signal amplification.

In at least one aspect, the system is directed towards detecting a nucleic acid target comprising a first target sequence and a second target sequence. In some embodiments, the nucleic acid target is a DNA molecule. In certain embodiments, the nucleic acid is an RNA molecule.

In some embodiments, a system is described, including a first nucleotide comprising a first nucleotide sequence configured to reversibly hybridize to the first target sequence; a second nucleotide comprising a second nucleotide sequence configured to reversibly hybridize to the second target sequence; a first reporter comprising a first reporter moiety and a first reporter sequence coupled to the first reporter moiety; and a second reporter comprising a second reporter moiety and a second reporter sequence coupled to the second reporter moiety. In at least one embodiment, the first reporter sequence is configured to reversibly hybridize the first nucleotide sequence, the second reporter sequence is configured to reversibly hybridize the second nucleotide sequence, and/or the second reporter sequence is configured to reversibly hybridize to the first reporter sequence. In at least one embodiment, the first nucleotide and the second nucleotide are configured to dimerize and/or the first reporter and the second reporter are configured to dimerize. In at least one embodiment, reversible hybridization of the first reporter sequence to the second reporter sequence is configured to bring the first reporter moiety and second reporter moiety into proximity. In some embodiments, reversible hybridization of one or more of the first reporter sequence to the first nucleotide sequence and the second reporter sequence to the second nucleotide sequence is configured to separate (bring out of proximity) the first reporter moiety and the second reporter moiety.

The reporter moieties are in proximity at any suitable distance. In some embodiments the reporter moieties are in proximity when they are about 0.1 nm to about 20 nm, about 0.5 nm to about 15 nm, about 1 nm to about 10 nm, about 2 nm to about 10 nm, about 1 nm, about 2 nm, about 3 nm, about 4 nm, about 5 nm, about 6 nm, about 7 nm, about 8 nm, about 9 nm, about 10 nm, about 11 nm, about 12 nm about 13 nm about 14 nm, or about 15 nm from one another. In some embodiments, both of the reporter moieties are fluorophores, and they are in proximity when they are about 1 nm to about 10 nm, 1 nm, about 2 nm, about 3 nm, about 4 nm, about 5 nm, about 6 nm, about 7 nm, about 8 nm, about 9 nm, or about 10 nm from one another. In some embodiments, one of the reporter moieties is a fluorophore and another reporter moiety is a quencher, and they are in proximity when they are about 1 nm to about 10 nm, about 2 nm to about 10 nm, about 1 nm, about 2 nm, about 3 nm, about 4 nm, about 5 nm, about 6 nm, about 7 nm, about 8 nm, about 9 nm, or about 10 nm from one another.

In certain embodiments, the first reporter moiety and the second reporter moiety provide a fluorescent signal that increases or decreases at a predetermined wavelength. A change in signal may be due to, for example, Förster resonance energy transfer (FRET) or quenching or de-quenching of a fluorophore by a quencher. Suitable fluorophore pairs known in the art for producing FRET signals are contemplated. In embodiments where the change is due to FRET, the first reporter moiety and the second reporter moiety may both be fluorophores. In embodiments where the change is due to quenching or de-quenching, the first reporter moiety may be a fluorophore (e.g., 6-Carboxyfluorescein, FAM) and the second moiety may be a quenching moiety (e.g., BHQ1). Other suitable fluorophore-quencher pairs known in the art for producing de-quenching signals are contemplated.

In certain embodiments, one or more of the first nucleotide and the second nucleotide are DNA molecules. In certain embodiments, one or more of the first nucleotide and the second nucleotide are RNA molecules. In some embodiments, the first nucleotide is a DNA molecule, and the second nucleotide is a DNA molecule. In at least one embodiment where the first nucleotide and the second nucleotide are DNA molecules, the first nucleotide comprises a first thymine base and the second nucleotide comprises a second thymine base. In at least one embodiment, the first thymine base and the second thymine base are configured to be brought into proximity by reversible hybridization of the first nucleotide sequence to the first target sequence and the second nucleotide sequence to the second target sequence. In at least one embodiment, the first thymine base and the second thymine base are configured to dimerize. In some embodiments, the first thymine base and the second thymine base are configured to dimerize when exposed to ultraviolet light. The thymine bases or other bases are in proximity at any suitable distance. In some embodiments the thymine bases or other bases are in proximity when they are about one base pair distance from one another. In some embodiments, the thymine bases or other bases are in proximity when they are about 0.1 nm to about 1 nm, about 0.1 nm to about 0.75 nm, about 0.1 nm to about 0.5 nm, about 0.25 nm to about 0.5 nm, or about 0.1 nm to about 0.25 nm from one another. In some embodiments, the thymine bases or other bases are in proximity when they are about 0.5 nm from one another. In some embodiments, the thymine bases or other bases are in proximity when they are about 0.25 nm from one another. In some embodiments, the thymine bases or other bases are in proximity when they are about 0.3 nm to about 0.4 nm from one another. In some embodiments, the thymine bases or other bases are in proximity when they are about 0.35 nm from one another.

In certain embodiments, the first nucleotide sequence of the first nucleotide is complementary to the first target sequence. In certain embodiments, the second nucleotide sequence of the second nucleotide is complementary to the second target sequence. In certain embodiments, one or more of the complementarities between the first nucleotide sequence and the first target sequence and between the second nucleotide sequence and the second target sequence are perfectly complementary, imperfectly complementary, or semi-complementary. Methods of producing imperfect or semi-complementary sequences include, but are not limited to, abasic sites and mismatches in the sequences. Sequences may comprise both one or more abasic sites and/or one or more mismatches to control for the complementarity of the sequence.

In certain embodiments, one or more of the first nucleotide and the second nucleotide comprises one or more abasic sites. An abasic site may also be referred to as an apurinic or apyrimidinic site. In some embodiments, at an abasic site there is neither a purine nor a pyrimidine base, though the phosphate backbone of the RNA or DNA is still present. It is understood that by introducing an abasic site at, e.g., the 5′ end of a nucleotide, hybridization between the nucleotide and another nucleic acid may be destabilized. Such destabilization encourages dissociation between two reversibly hybridizable nucleic acids.

In certain embodiments, the first nucleotide sequence and the first target sequence comprise one or more mismatched bases. In certain embodiments, the second nucleotide sequence and the second target sequence comprise one or more mismatched bases. A matched base, or complementary base readily hybridize or base pair. For example, adenine and thymine hybridize, adenine and uracil hybridize, and guanine and cytosine hybridize. Mismatched bases include, but are not limited to, adenine and adenine, adenine and guanine, adenine and cytosine, thymine and thymine, thymine and uracil, thymine and guanine, thymine and cytosine, uracil and uracil, uracil and guanine, uracil and cytosine, guanine and guanine, and cytosine and cytosine. It is understood that by introducing one or more mismatched base the nucleotide and another nucleic acid will be destabilized. Such destabilization encourages dissociation between two reversibly hybridizable nucleic acids. Generally, the destabilization resulting from a mismatched base pair is less than the destabilization resulting from an abasic site.

In certain embodiments, one or more of the first reporter and the second reporter are DNA molecules. In certain embodiments, one or more of the first reporter and the second reporter are RNA molecules. In some embodiments, the first reporter is a DNA molecule, and the second reporter is a DNA molecule. In at least one embodiment where the first reporter and the second reporter are DNA molecules, the first reporter comprises a first thymine base and the second reporter comprises a second thymine base. In at least one embodiment, the first thymine base and the second thymine base are configured to be brought into proximity by reversible hybridization of the first reporter sequence to the first nucleotide sequence and the second reporter sequence to the second nucleotide sequence. In at least one embodiment, the first thymine base and the second thymine base are configured to dimerize. In some embodiments, the first thymine base and the second thymine base dimerize when exposed to ultraviolet light. The thymine bases or other bases are in proximity at any suitable distance. In some embodiments the thymine bases or other bases are in proximity when they are about one base pair distance from one another. In some embodiments, the thymine bases or other bases are in proximity when they are about 0.1 nm to about 1 nm, about 0.1 nm to about 0.75 nm, about 0.1 nm to about 0.5 nm, about 0.25 nm to about 0.5 nm, or about 0.1 nm to about 0.25 nm from one another. In some embodiments, the thymine bases or other bases are in proximity when they are about 0.5 nm from one another. In some embodiments, the thymine bases or other bases are in proximity when they are about 0.25 nm from one another. In some embodiments, the thymine bases or other bases are in proximity when they are about 0.3 nm to about 0.4 nm from one another. In some embodiments, the thymine bases or other bases are in proximity when they are about 0.35 nm from one another.

In certain embodiments, the first reporter sequence of the first reporter is complementary to the first nucleotide sequence. In certain embodiments, the second reporter sequence of the second reporter is complementary to the second nucleotide sequence. In certain embodiments, one or more of the complementarities between the first nucleotide sequence and the first reporter sequence and between the second nucleotide sequence and the second reporter sequence are perfectly complementary, imperfectly complementary, or semi-complementary. Methods of producing imperfect or semi-complementary sequences include, but are not limited to, abasic sites and mismatches in the sequences. Sequences may comprise both one or more abasic sites and one or more mismatches to control for the complementarity of the sequence.

In certain embodiments, one or more of the first reporter and the second reporter comprises one or more abasic sites. In certain embodiments, the first nucleotide sequence and the first reporter sequence comprise one or more mismatched bases. In certain embodiments, the second nucleotide sequence and the second reporter sequence comprise one or more mismatched bases.

In at least one aspect, a method of detecting a nucleic acid target is disclosed, including a first target sequence and a second target sequence. In at least one embodiment, the method comprises: providing a first nucleotide comprising a first nucleotide sequence; hybridizing the first nucleotide sequence to the first target sequence; providing a second nucleotide comprising a second nucleotide sequence; hybridizing the second nucleotide sequence to the second target sequence; dimerizing the first nucleotide and the second nucleotide to form a first dimer upon hybridizing the first nucleotide sequence to the first target sequence and hybridizing the second nucleotide sequence to the second target sequence; dissociating the first dimer and the nucleic acid target; providing a reporter complex comprising: a first reporter comprising a first reporter moiety and a first reporter sequence coupled to the first reporter moiety, wherein the first reporter moiety is configured to produce a first reporter moiety signal, and a second reporter comprising a second reporter moiety and a second reporter sequence coupled to the second reporter moiety, wherein the second reporter sequence is hybridized to the first reporter sequence, wherein the second reporter moiety is configured to alter the first reporter moiety signal when the first reporter moiety and the second reporter moiety are in proximity, and wherein hybridization of the first reporter sequence to the second reporter sequence is configured to bring the first reporter moiety and the second reporter moiety into proximity; dissociating the first reporter sequence and the second reporter sequence; hybridizing the first reporter sequence to the first nucleotide sequence; hybridizing the second reporter sequence to the second nucleotide sequence; bringing the first reporter moiety and the second reporter moiety out of proximity by hybridizing the first reporter sequence to the first nucleotide sequence of the first dimer and/or hybridizing the second reporter sequence to the second nucleotide sequence of the first dimer, and detecting a change in the first reporter moiety signal. In some embodiments, the hybridizations are reversible.

In some embodiments, the first nucleotide comprises a first nucleotide thymine base and the second nucleotide comprises a second nucleotide thymine base. In some embodiments, dimerization of the first nucleotide and the second nucleotide comprises bringing the first nucleotide thymine base into proximity with the second nucleotide thymine base, providing an ultraviolet light source, and forming one or more bonds coupling the first nucleotide thymine base and the second nucleotide thymine base. Other bases (e.g., other pyrimidines such as uracil or cytosine) may also be dimerized by forming bonds upon exposure to an ultraviolet light source. Other non-limiting dimerization conditions include enzymatic dimerization (e.g., the use of a ligase) and chemical dimerization (e.g., chemical crosslinking). The thymine bases or other bases are in proximity at any suitable distance. In some embodiments the thymine bases or other bases are in proximity when they are about one base pair distance from one another. In some embodiments, the thymine bases or other bases are in proximity when they are about 0.1 nm to about 1 nm, about 0.1 nm to about 0.75 nm, about 0.1 nm to about 0.5 nm, about 0.25 nm to about 0.5 nm, or about 0.1 nm to about 0.25 nm from one another. In some embodiments, the thymine bases or other bases are in proximity when they are about 0.5 nm from one another. In some embodiments, the thymine bases or other bases are in proximity when they are about 0.25 nm from one another. In some embodiments, the thymine bases or other bases are in proximity when they are about 0.3 nm to about 0.4 nm from one another. In some embodiments, the thymine bases or other bases are in proximity when they are about 0.35 nm from one another.

In some embodiments, the method further involved dimerizing the first reporter and the second reporter to form a reporter dimer. In some embodiments, the first reporter comprises a first reporter thymine base and the second reporter comprises a second reporter thymine base. In some embodiments, dimerization of the first reporter and the second reporter comprises bringing the first reporter thymine base into proximity with the second reporter thymine base, providing an ultraviolet light source, and forming one or more bonds coupling the first reporter thymine base and the second reporter thymine base. Other bases (e.g., other pyrimidines such as uracil or cytosine) may also be dimerized by forming bonds upon exposure to an ultraviolet light source. Other non-limiting dimerization conditions include enzymatic dimerization (e.g., the use of a ligase) and chemical dimerization (e.g., chemical crosslinking). The thymine bases or other bases are in proximity at any suitable distance. In some embodiments the thymine bases or other bases are in proximity when they are about one base pair distance from one another. In some embodiments, the thymine bases or other bases are in proximity when they are about 0.1 nm to about 1 nm, about 0.1 nm to about 0.75 nm, about 0.1 nm to about 0.5 nm, about 0.25 nm to about 0.5 nm, or about 0.1 nm to about 0.25 nm from one another. In some embodiments, the thymine bases or other bases are in proximity when they are about 0.5 nm from one another. In some embodiments, the thymine bases or other bases are in proximity when they are about 0.25 nm from one another. In some embodiments, the thymine bases or other bases are in proximity when they are about 0.3 nm to about 0.4 nm from one another. In some embodiments, the thymine bases or other bases are in proximity when they are about 0.35 nm from one another.

In some embodiments, the reporter dimer is dissociated from the first dimer. It will be readily understood that following dissociation from a first reporter dimer, the first dimer can hybridize another first reporter and another second reporter. This other first reporter and other second reporter will also provide a signal once hybridized to the first dimer, and this other first reporter and other second reporter can also be dimerized while hybridized to the first dimer. In this manner, the signal may be amplified (e.g., exponentially).

In some embodiments, detection of the change in a signal produced by one or more of the first reporter moiety and the second reporter moiety comprises detecting one or more of an increase in fluorescence at a first predetermined wavelength and a decrease in fluorescence at a second predetermined wavelength as a result of the reporter moieties coming into or being brought out of proximity to one another. Detection of the increase and/or decrease in fluorescence may be achieved by using any suitable technique. Suitable techniques include, but are not limited to, Förster resonance energy transfer (FRET), and fluorophore quenching or de-quenching. Suitable fluorophore pairs known in the art for producing FRET signals are contemplated. Suitable fluorophore-quencher pairs known in the art for producing de-quenching signals are contemplated.

In at least one aspect, a system for detecting a nucleic acid target including a first target sequence and a second target sequence is disclosed. In some embodiments, the nucleic acid target is a DNA molecule. In certain embodiments, the nucleic acid is an RNA molecule.

In some embodiments, a system is disclosed, including a first nucleotide comprising a first nucleotide sequence configured to reversibly hybridize to the first target sequence; a second nucleotide comprising a second nucleotide sequence configured to reversibly hybridize to the second target sequence; a first probe comprising a first probe sequence and a second probe sequence; a second probe comprising a third probe sequence and a fourth probe sequence; and a reporter comprising: a first reporter sequence configured to reversibly hybridize the second probe sequence, a second reporter sequence coupled to the first reporter sequence, a first reporter moiety coupled to first reporter sequence, and a second reporter moiety coupled to the second reporter sequence. In some embodiments, the first probe sequence is configured to reversibly hybridize the first nucleotide sequence. In some embodiments, the third probe sequence is configured to reversibly hybridize the second nucleotide sequence. In some embodiments, the second reporter sequence is configured to reversibly hybridize the fourth probe sequence. In some embodiments, the second reporter sequence is configured to reversibly hybridize to the first reporter sequence. In some embodiments, the first nucleotide and the second nucleotide are configured to dimerize. In some embodiments, reversible hybridization of the first reporter sequence to the second reporter sequence is configured to bring the first reporter moiety and second reporter moiety into proximity. In some embodiments, reversible hybridization of one or more of the first reporter sequence to the second probe sequence and the second reporter sequence to the fourth probe sequence is configured to separate (bring out of proximity) the first reporter moiety and the second reporter moiety.

The reporter moieties are in proximity at any suitable distance. In some embodiments the reporter moieties are in proximity when they are about 0.1 nm to about 20 nm, about 0.5 nm to about 15 nm, about 1 nm to about 10 nm, about 2 nm to about 10 nm, about 1 nm, about 2 nm, about 3 nm, about 4 nm, about 5 nm, about 6 nm, about 7 nm, about 8 nm, about 9 nm, about 10 nm, about 11 nm, about 12 nm about 13 nm about 14 nm, or about 15 nm from one another. In some embodiments, both of the reporter moieties are fluorophores, and they are in proximity when they are about 1 nm to about 10 nm, 1 nm, about 2 nm, about 3 nm, about 4 nm, about 5 nm, about 6 nm, about 7 nm, about 8 nm, about 9 nm, or about 10 nm from one another. In some embodiments, one of the reporter moieties is a fluorophore and another reporter moiety is a quencher, and they are in proximity when they are about 1 nm to about 10 nm, about 2 nm to about 10 nm, about 1 nm, about 2 nm, about 3 nm, about 4 nm, about 5 nm, about 6 nm, about 7 nm, about 8 nm, about 9 nm, or about 10 nm from one another.

In certain embodiments, one or more of the first nucleotide and the second nucleotide are DNA molecules. In certain embodiments, one or more of the first nucleotide and the second nucleotide are RNA molecules. In some embodiments, the first nucleotide is a DNA molecule, and the second nucleotide is a DNA molecule. In at least one embodiment where the first nucleotide and the second nucleotide are DNA molecules, the first nucleotide comprises a first thymine base and the second nucleotide comprises a second thymine base. In at least one embodiment, the first thymine base and the second thymine base are configured to be brought into proximity by reversible hybridization of the first nucleotide sequence to the first target sequence and the second nucleotide sequence to the second target sequence. In at least one embodiment, the first thymine base and the second thymine base are configured to dimerize when exposed to ultraviolet light. The thymine bases or other bases are in proximity at any suitable distance. In some embodiments the thymine bases or other bases are in proximity when they are about one base pair distance from one another. In some embodiments, the thymine bases or other bases are in proximity when they are about 0.1 nm to about 1 nm, about 0.1 nm to about 0.75 nm, about 0.1 nm to about 0.5 nm, about 0.25 nm to about 0.5 nm, or about 0.1 nm to about 0.25 nm from one another. In some embodiments, the thymine bases or other bases are in proximity when they are about 0.5 nm from one another. In some embodiments, the thymine bases or other bases are in proximity when they are about 0.25 nm from one another. In some embodiments, the thymine bases or other bases are in proximity when they are about 0.3 nm to about 0.4 nm from one another. In some embodiments, the thymine bases or other bases are in proximity when they are about 0.35 nm from one another.

In certain embodiments, the first nucleotide sequence of the first nucleotide is complementary to the first target sequence. In certain embodiments, the second nucleotide sequence of the second nucleotide is complementary to the second target sequence. In certain embodiments, one or more of the complementarities between the first nucleotide sequence and the first target sequence and between the second nucleotide sequence and the second target sequence are imperfectly complementary or semi-complementary. Methods of producing imperfect or semi-complementary sequences include, but are not limited to, including abasic sites and mismatches in the sequences. Sequences may comprise both one or more abasic sites and one or more mismatches to control for the complementarity of the sequence.

In certain embodiments, one or more of the first nucleotide and the second nucleotide comprises one or more abasic sites. An abasic site may also be referred to as an apurinic or apyrimidinic site. At an abasic site there is neither a purine nor a pyrimidine base, though the phosphate backbone of the RNA or DNA is still present. It is understood that by introducing an abasic site at, e.g., the 5′ end of a nucleotide hybridization between the nucleotide and another nucleic acid will be destabilized. Such destabilization encourages dissociation between two reversibly hybridizable nucleic acids.

In certain embodiments, the first nucleotide sequence and the first target sequence comprise one or more mismatched bases. In certain embodiments, the second nucleotide sequence and the second target sequence comprise one or more mismatched bases. A matched base, or complementary base readily hybridize or base pair. For example, adenine and thymine hybridize, adenine and uracil hybridize, and guanine and cytosine hybridize. Mismatched bases include, but are not limited to, adenine and adenine, adenine and guanine, adenine and cytosine, thymine and thymine, thymine and uracil, thymine and guanine, thymine and cytosine, uracil and uracil, uracil and guanine, uracil and cytosine, guanine and guanine, and cytosine and cytosine. It is understood that by introducing one or more mismatched base the nucleotide and another nucleic acid will be destabilized. Such destabilization encourages dissociation between two reversibly hybridizable nucleic acids. Generally, the destabilization resulting from a mismatched base pair is less than the destabilization resulting from an abasic site.

In certain embodiments, oner or more of the first probe and the second probe is a DNA molecule. In certain embodiments, one or more of the second probe is an RNA molecule. In some embodiments, the first probe is a DNA molecule and the second probe is a DNA molecule. In at least on embodiment where the first probe is a DNA molecule, the first comprises a first thymine base and the second probe comprises a second thymine base. In at least one embodiment, the first thymine base and the second thymine base are configured to be brought into proximity by reversible hybridization of the first nucleotide sequence to the first probe sequence and the second nucleotide sequence to the third probe sequence. In at least one embodiment, the first thymine base and the second thymine base are configured to dimerize when exposed to ultraviolet light. The thymine bases or other bases are in proximity at any suitable distance. In some embodiments the thymine bases or other bases are in proximity when they are about one base pair distance from one another. In some embodiments, the thymine bases or other bases are in proximity when they are about 0.1 nm to about 1 nm, about 0.1 nm to about 0.75 nm, about 0.1 nm to about 0.5 nm, about 0.25 nm to about 0.5 nm, or about 0.1 nm to about 0.25 nm from one another. In some embodiments, the thymine bases or other bases are in proximity when they are about 0.5 nm from one another. In some embodiments, the thymine bases or other bases are in proximity when they are about 0.25 nm from one another. In some embodiments, the thymine bases or other bases are in proximity when they are about 0.3 nm to about 0.4 nm from one another. In some embodiments, the thymine bases or other bases are in proximity when they are about 0.35 nm from one another.

In certain embodiments, the reporter is a DNA molecule. In certain embodiments, the reporter is an RNA molecule.

In certain embodiments, the first reporter sequence is complementary to the second reporter sequence. In certain embodiments, the complementarity between the first reporter sequence and the second reporter sequence is imperfectly complementary or semi-complementary. Methods of producing imperfect or semi-complementary sequences include, but are not limited to, including abasic sites and mismatches in the sequences. Sequences may comprise both one or more abasic sites and one or more mismatches to control for the complementarity of the sequence.

In certain embodiments, the first probe sequence is complementary to the first nucleotide sequence. In certain embodiments, the second probe sequence is complementary to the first reporter sequence. In certain embodiments, the third probe sequence is complementary to the second nucleotide sequence. In certain embodiments, the fourth probe sequence is complementary to the second reporter sequence. In certain embodiments, the complementary between one or more of the first probe sequence and the first nucleotide sequence, the second probe sequence and the first reporter sequence, the third probe sequence and the second nucleotide sequence, and the fourth probe sequence and the second reporter sequence is imperfectly complementary or semi-complementary. Methods of producing imperfect or semi-complementary sequences include, but are not limited to, including abasic sites and mismatches in the sequences. Sequences may comprise both one or more abasic sites and one or more mismatches to control for the complementarity of the sequence. For example, the first probe sequence may comprise one or more mismatch bases compared to the first nucleotide sequence, the second probe sequence may comprise one or more mismatch bases compared to the first reporter sequence, the third probe sequence may comprise one or more mismatch bases compared to the second nucleotide sequence, and/or the fourth probe sequence may comprise one or more mismatch bases compared to the second reporter sequence. As a further example, one or more of the first probe sequence, the first nucleotide sequence, the second probe sequence, the first reporter sequence, the third probe sequence, the second nucleotide sequence, and/or the fourth probe sequence may comprise one or more abasic sites.

In certain embodiments, the first reporter moiety and the second reporter moiety provide a fluorescent signal that increases or decreases at a predetermined wavelength. Any moiety that can produce a signal (e.g., a fluorophore) or alter (e.g., a quencher) may be used. In some embodiments, at least one moiety is a fluorophore that produces a signal (e.g., a fluorescent emission at a wavelength) when provided a stimulus (e.g., a light source at a different wavelength). In some embodiments, both moieties are fluorophores, and the second moiety provides a signal in response to the first moieties signal (e.g., FRET). A change in signal may be due to, for example, Förster resonance energy transfer (FRET) or quenching or de-quenching of a fluorophore by a quencher. Suitable fluorophore pairs known in the art for producing FRET signals are contemplated. In embodiments where the change is due to FRET, the first reporter moiety and the second reporter moiety may both be fluorophores. In embodiments where the change is due to quenching or de-quenching, the first moiety may be a fluorophore (e.g., 6-Carboxyfluorescein, FAM) and the second moiety may be a quenching moiety (e.g., BHQ1). Other suitable fluorophore-quencher pairs known in the art for producing de-quenching signals are contemplated.

In at least another aspect, a method of detecting a nucleic acid target comprising a first target sequence and a second target sequence is described, comprises providing a first nucleotide comprising a first nucleotide sequence; hybridizing the first nucleotide sequence to the first target sequence; providing a second nucleotide comprising a second nucleotide sequence; hybridizing the second nucleotide sequence to the second target sequence; dimerizing the first nucleotide and the second nucleotide to form a first dimer upon reversibly hybridizing the first nucleotide sequence to the first target sequence and reversibly hybridizing the second nucleotide sequence to the second target sequence; dissociating the first dimer and the nucleic acid target; providing a first probe comprising a first probe sequence and a second probe sequence; hybridizing the first probe sequence to the first nucleotide sequence; providing a second probe comprising a third probe sequence and a fourth probe sequence; hybridizing the third probe sequence to the second nucleotide sequence; provide a reporter comprising: a first reporter moiety configured to produce a first reporter moiety signal, a first reporter sequence coupled to the first reporter moiety, wherein the first reporter sequence is configured to reversibly hybridize the second probe sequence, a second reporter moiety configured to alter the first reporter moiety signal when the first reporter moiety and the second reporter moiety are in proximity, and a second reporter sequence, wherein the second reporter sequence is coupled to the second reporter moiety, wherein the second reporter sequence is coupled to the first reporter sequence, wherein the second reporter sequence is reversibly hybridized to the first reporter sequence, wherein the second reporter sequence is configured to reversibly hybridize the second nucleotide sequence, and wherein reversible hybridization of the first reporter sequence to the second reporter sequence is configured to bring the first reporter moiety and the second reporter moiety into proximity; and dissociating the first reporter sequence and the second reporter sequence; hybridizing the first reporter sequence to the second probe sequence; hybridizing the second reporter sequence to the fourth probe sequence; bringing the first reporter moiety and the second reporter moiety out of proximity by reversible hybridizing the first reporter sequence to the second probe sequence and/or reversible hybridizing the second reporter sequence to the fourth probe sequence, and detecting a change in the first reporter moiety signal. In at least one embodiment, the second reporter sequence is hybridized to the first reporter sequence before it is provided.

In some embodiments, the first nucleotide comprises a first nucleotide thymine base and the second nucleotide comprises a second nucleotide thymine base. In some embodiments, dimerization of the first nucleotide and the second nucleotide comprises bringing the first nucleotide thymine base into proximity with the second nucleotide thymine base, providing an ultraviolet light source, and forming one or more bonds coupling the first nucleotide thymine base and the second nucleotide thymine base. Other bases (e.g., other pyrimidines such as uracil or cytosine) may also be dimerized by forming bonds upon exposure to an ultraviolet light source. The thymine bases or other bases are in proximity at any suitable distance. In some embodiments the thymine bases or other bases are in proximity when they are about one base pair distance from one another. In some embodiments, the thymine bases or other bases are in proximity when they are about 0.1 nm to about 1 nm, about 0.1 nm to about 0.75 nm, about 0.1 nm to about 0.5 nm, about 0.25 nm to about 0.5 nm, or about 0.1 nm to about 0.25 nm from one another. In some embodiments, the thymine bases or other bases are in proximity when they are about 0.5 nm from one another. In some embodiments, the thymine bases or other bases are in proximity when they are about 0.25 nm from one another. In some embodiments, the thymine bases or other bases are in proximity when they are about 0.3 nm to about 0.4 nm from one another. In some embodiments, the thymine bases or other bases are in proximity when they are about 0.35 nm from one another.

In some embodiments, the method further involved dimerizing the first probe and the probe to form a probe dimer. In some embodiments, the first probe comprises a first probe thymine base and the second probe comprises a second probe thymine base. In some embodiments, dimerization of the first probe and the second probe comprises bringing the first probe thymine base into proximity with the second probe thymine base, providing an ultraviolet light source, and forming one or more bonds coupling the first probe thymine base and the second probe thymine base. Other bases (e.g., other pyrimidines such as uracil or cytosine) may also be dimerized by forming bonds upon exposure to an ultraviolet light source. The thymine bases or other bases are in proximity at any suitable distance. In some embodiments the thymine bases or other bases are in proximity when they are about one base pair distance from one another. In some embodiments, the thymine bases or other bases are in proximity when they are about 0.1 nm to about 1 nm, about 0.1 nm to about 0.75 nm, about 0.1 nm to about 0.5 nm, about 0.25 nm to about 0.5 nm, or about 0.1 nm to about 0.25 nm from one another. In some embodiments, the thymine bases or other bases are in proximity when they are about 0.5 nm from one another. In some embodiments, the thymine bases or other bases are in proximity when they are about 0.25 nm from one another. In some embodiments, the thymine bases or other bases are in proximity when they are about 0.3 nm to about 0.4 nm from one another. In some embodiments, the thymine bases or other bases are in proximity when they are about 0.35 nm from one another.

In some embodiments, the probe dimer is dissociated from the first dimer. It will be readily understood that following dissociation from a first probe dimer, the first dimer can hybridize another first probe and another second probe. This other first probe and other second probe can also be dimerized while hybridized to the first dimer. In this manner, the signal may be amplified (e.g., exponentially).

In some embodiments, detection of the change in a signal produced by one or more of the first reporter moiety and the second reporter moiety comprises detecting one or more of an increase in fluorescence at a first predetermined wavelength and a decrease in fluorescence at a second predetermined wavelength. Detection of the increase and/or decrease in fluorescence may be achieving by any suitable technique. Suitable techniques include, but are not limited to, Forster resonance energy transfer (FRET), and fluorophore quenching or de-quenching. Suitable fluorophore pairs known in the art for producing FRET signals are contemplated. Suitable fluorophore-quencher pairs known in the art for producing de-quenching signals are contemplated.

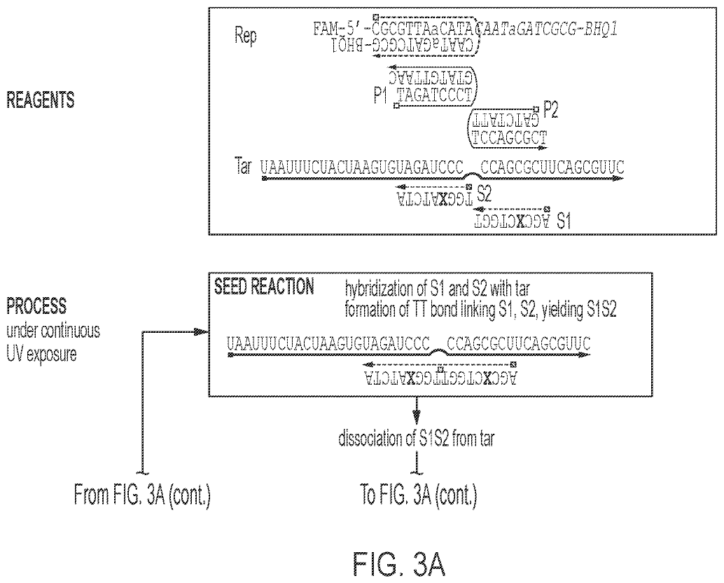

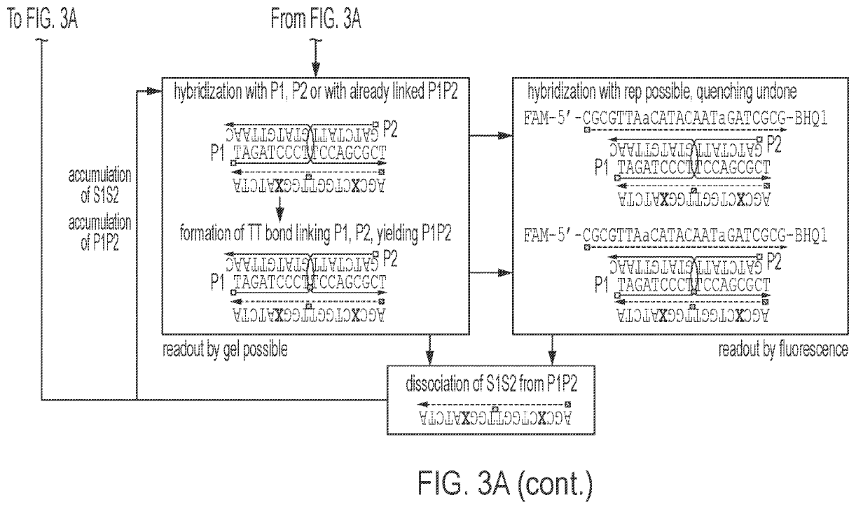

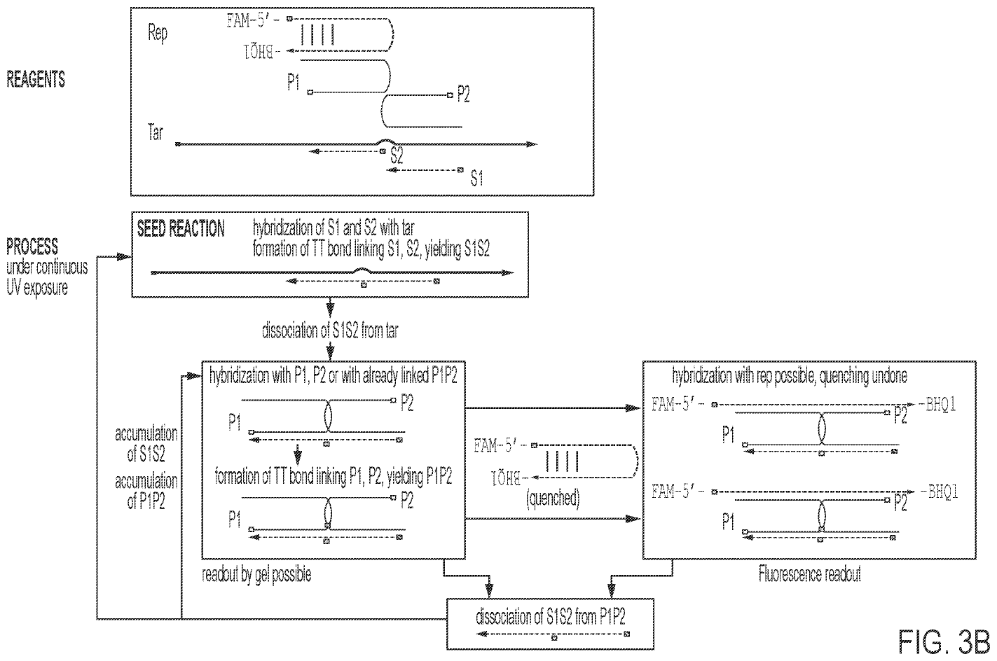

FIGS. 3A and 3B depict exemplary embodiments of an aspect of the system or method. FIG. 3A depicts an exemplary embodiment of the system or method to detect the N gene gRNA of the SARS-CoV-2 virus with the sequence:

(SEQ ID NO: 1)
5′-UAAUUUCUACUAAGUGUAGAUCCCCCAGCGCUUCA GCGUUC-3′.

In FIG. 3A two nucleotides are introduced (S1 (5′-AGCXCTGGT-3′) and S2 (5′-TGGXATCTA-3′)). The two nucleotides are semi-complementary to two separate sequences present on the SARS-CoV-2 virus target (Tar). Each of the two nucleotides in the example possess an abasic or mismatch base (X). The 5′ end of one nucleotide (S2) is a thymine base (T). The 3′ end of the other nucleotide (S1) is a thymine base (T). The two nucleotides reversibly hybridize to the target SARS-CoV-2 target (Tar). Upon hybridization to the target, the 3′ thymine of S2 and the 5′ thymine of S1 are held in proximity to one another. Exposure of the S1-Tar-S2 complex to an ultraviolet light results in dimerization between the two nucleotide thymines (black square) dimerizing the two nucleotides (S1S2). The dimerized S1S2 nucleic acid dissociates from (melts off of) the target SARS-CoV-2 (Tar). The dissociated target is free to complex another pair of non-dimerized first and second nucleotides, providing signal amplification.

Following dissociation, the nucleotide dimer (S1S2) hybridizes to two semi-complementary probes (P1 and P2). The first probe (P1) comprises a first sequence (5′-TAGATCCCT-3′) semi-complementary to the sequence of the second nucleotide and a second sequence (5′GTATGTTAAC-3′ (SEQ ID NO: 2)) complementary to a sequence of the 5′ end of the reporter (Rep). The second probe (P2) comprises a first sequence (5′-TCCAGCGCT-3′) semi-complementary to the sequence of the first nucleotide and a second sequence (5′-GATCTATT-3′) complementary to a sequence of the 3′ end of the reporter (Rep). Upon hybridization to the S1S2 nucleotide dimer, an internal thymine (T) in the first probe (P1) and another internal thymine (T) in the second probe (P2) are brought into proximity with each other. Exposure of the P1-S1S2-P2 complex to an ultraviolet light results in dimerization between the two internal probe thymines (square) dimerizing the two probes (P1P2). The dimerized S1S2 nucleic acid dissociates from (melts off of) the P1P2 probe dimer. The dissociated S1S2 nucleic acid is free to complex another pair of non-dimerized first and second probes, providing further signal amplification.

Following dissociation, the probe dimer (P1P2) is free to hybridize a self-hybridizing reporter. The reporter comprises a first sequence (5′-CGCGTTAaCATA-3′ (SEQ ID NO: 3)) and a second sequence (5′-CAATaGATCGCG-3′ (SEQ ID NO: 4)) and is semi-self-complementary with a base pair mismatch between two adenines (a). The first reporter sequence is complementary to the second sequence of the first probe. The second reporter sequence is complementary to the second sequence of the second probe. Hybridization of the first reporter sequence to the first sequence of the first probe and hybridization of the second reporter sequence to the second sequence of the second probe creates a P1P2-Rep complex. The P1P2-Rep complex distances the 5′ end of the reporter molecule from the 3′ end of the reporter molecule, thereby distancing a fluorescent moiety coupled to the 5′ end of the reporter molecule (FAM) from a quenching moiety coupled to the 3′ end of the reporter molecule (BHQ1). This separation (bringing out of proximity) leads to the de-quenching of the fluorescent moiety providing a detectable increase in fluorescence.

FIG. 3B depicts a generalized embodiment of the system to detect a nucleic acid of interest.

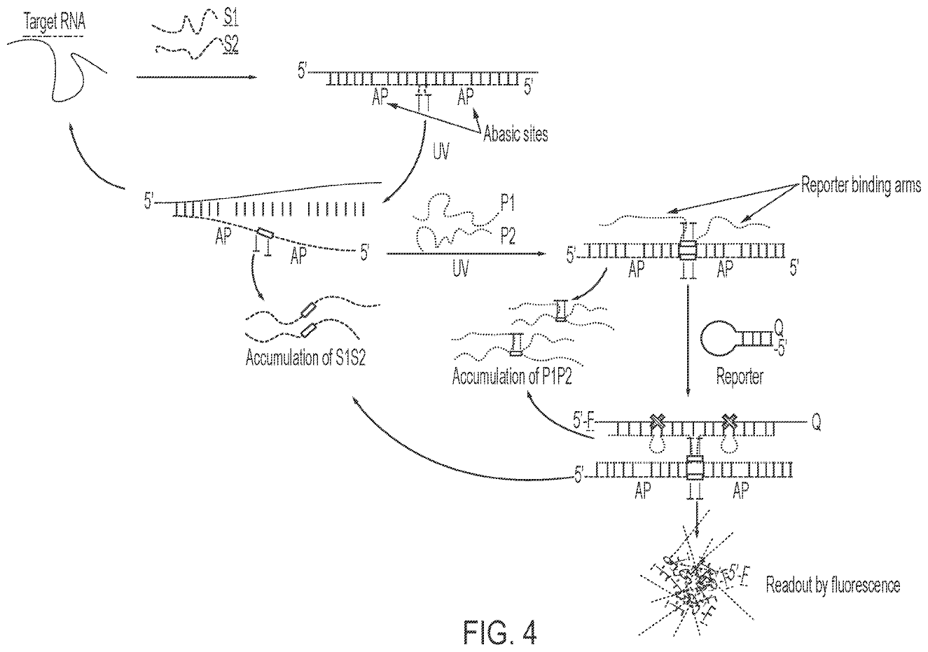

In some embodiments, the fluorescent moiety is not re-quenched upon dissociation of the probe dimer from the reporter. FIG. 4 depicts one such embodiment. In some embodiments where the fluorescent moiety of the reporter is not re-quenched upon dissociation, the dissociated P1P2 probe dimer is free to complex another self-hybridized reporter, providing further signal amplification.

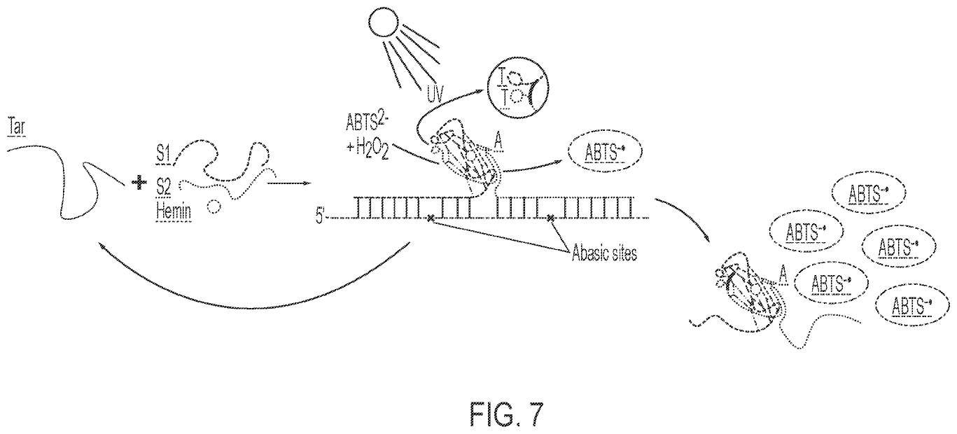

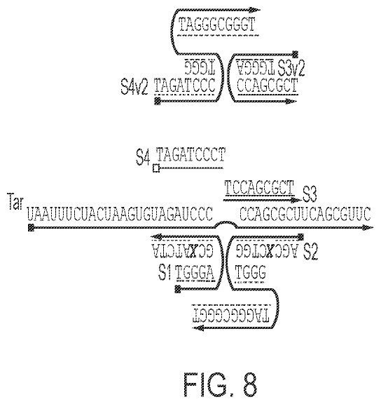

In at least another aspect, the system or method disclosed herein is based on a Deoxyribozyme (DNAzyme) or Ribozyme (RNAzyme). In some embodiments, the Deoxyribozyme (DNAzyme) is a peroxidase-mimicking G-quadruplex Deoxyribozyme (DNAzyme) that catalyzes generation of a colorimetric signal. Any suitable Deoxyribozyme (DNAzyme) or Ribozyme (RNAzyme) that is capable of producing a detectable signal may be used. The system or method may include any cofactors (e.g., hemin). In some embodiments, the Deoxyribozyme (DNAzyme) or Ribozyme (RNAzyme) is split at a site that can be dimerized (e.g., at two neighboring thymines). For example, FIGS. 7 and 8 depict an exemplary embodiment. In the depicted embodiment, the split site is between two adjacent thymines. These thymines are dimerizable in the presence of ultraviolet light. In some embodiments, the Deoxyribozyme (DNAzyme) activity may be boosted by adding a 3′ terminal adenine base. In some embodiments, the system or method provides for exponential signal amplification.

In at least one aspect, the system for a nucleic acid target comprising a first target sequence and a second target sequence is disclosed. In some embodiments, the nucleic acid target is a DNA molecule. In some embodiments, the nucleic acid target is an RNA molecule.

In some embodiments, a system is disclosed, comprising a first nucleotide comprising a first nucleotide sequence and a first enzymatic sequence coupled to the first nucleotide sequence; a second nucleotide comprising a second nucleotide sequence and a second enzymatic sequence coupled to the second nucleotide sequence; and one or more substrates. In some embodiments, the first nucleotide sequence is configured to reversibly hybridize to the first target sequence. In some embodiments, the second nucleotide sequence is configured to reversibly hybridize to the second target sequence. In some embodiments, the first nucleotide and the second nucleotide are configured to dimerize. In some embodiments, the dimerized first nucleotide and second nucleotide is configured to convert the one or more substrates into one or more products. In some embodiments, the system further comprises a first seed nucleotide comprising a first seed sequence configured to reversibly hybridize to the first nucleotide sequence; and a second seed nucleotide comprising a second seed sequence configured to reversibly hybridize to the second nucleotide sequence. In some embodiments, the first seed nucleotide and the second seed nucleotide are configured to dimerize.

In some embodiments, the system further comprises a third nucleotide comprising a third nucleotide sequence and a third enzymatic sequence coupled to the third nucleotide sequence; and a fourth nucleotide comprising a fourth nucleotide sequence and a fourth enzymatic sequence coupled to the fourth nucleotide sequence. In some embodiments, the third nucleotide sequence is configured to reversibly hybridize to the first nucleotide sequence wherein the fourth nucleotide sequence is configured to reversibly hybridize to the second nucleotide sequence. In some embodiments, the third nucleotide and the fourth nucleotide are configured to dimerize. In some embodiments, the dimerized third nucleotide and fourth nucleotide is configured to convert the one or more substrates into one or more products.

In some embodiments, one or more of the first nucleotide, the second nucleotide, the third nucleotide, and the fourth nucleotide is a DNA molecule. In some embodiments, one or more of the first nucleotide, the second nucleotide, the third nucleotide, and the fourth nucleotide is an RNA molecule.

In some embodiments, the first nucleotide is a DNA molecule, the second nucleotide is a DNA molecule, the first nucleotide comprises a first thymine base, the second nucleotide comprises a second thymine base, the first thymine base and the second thymine base are configured to be brought into proximity by reversible hybridization of the first nucleotide sequence to the first target sequence and the second nucleotide sequence to the second target sequence, and the first thymine base and the second thymine base are configured to dimerize when exposed to ultraviolet light. The thymine bases or other bases are in proximity at any suitable distance. In some embodiments the thymine bases or other bases are in proximity when they are about one base pair distance from one another. In some embodiments, the thymine bases or other bases are in proximity when they are about 0.1 nm to about 1 nm, about 0.1 nm to about 0.75 nm, about 0.1 nm to about 0.5 nm, about 0.25 nm to about 0.5 nm, or about 0.1 nm to about 0.25 nm from one another. In some embodiments, the thymine bases or other bases are in proximity when they are about 0.5 nm from one another. In some embodiments, the thymine bases or other bases are in proximity when they are about 0.25 nm from one another. In some embodiments, the thymine bases or other bases are in proximity when they are about 0.3 nm to about 0.4 nm from one another. In some embodiments, the thymine bases or other bases are in proximity when they are about 0.35 nm from one another.

In certain embodiments, the first nucleotide sequence of the first nucleotide is complementary to the first target sequence. In certain embodiments, the second nucleotide sequence of the second nucleotide is complementary to the second target sequence. In certain embodiments, one or more of the complementarities between the first nucleotide sequence and the first target sequence and between the second nucleotide sequence and the second target sequence are imperfectly complementary or semi-complementary. Methods of producing imperfect or semi-complementary sequences include, but are not limited to, including abasic sites and mismatches in the sequences. Sequences may comprise both one or more abasic sites and one or more mismatches to control for the complementarity of the sequence.

In certain embodiments, one or more of the first nucleotide and the second nucleotide comprises one or more abasic sites. An abasic site may also be referred to as an apurinic or apyrimidinic site. At an abasic site there is neither a purine nor a pyrimidine base, though the phosphate backbone of the RNA or DNA is still present. It is understood that by introducing an abasic site at, e.g., the 5′ end of a nucleotide hybridization between the nucleotide and another nucleic acid will be destabilized. Such destabilization encourages dissociation between two reversibly hybridizable nucleic acids.

In some embodiments, the third nucleotide is a DNA molecule, the fourth nucleotide is a DNA molecule, the third nucleotide comprises a first thymine base, the fourth nucleotide comprises a second thymine base, the first thymine base and the second thymine base are configured to be brought into proximity by reversible hybridization of the third nucleotide sequence to the first nucleotide sequence and the fourth nucleotide sequence to the second nucleotide sequence, and the first thymine base and the second thymine base are configured to dimerize when exposed to ultraviolet light. The thymine bases or other bases are in proximity at any suitable distance. In some embodiments the thymine bases or other bases are in proximity when they are about one base pair distance from one another. In some embodiments, the thymine bases or other bases are in proximity when they are about 0.1 nm to about 1 nm, about 0.1 nm to about 0.75 nm, about 0.1 nm to about 0.5 nm, about 0.25 nm to about 0.5 nm, or about 0.1 nm to about 0.25 nm from one another. In some embodiments, the thymine bases or other bases are in proximity when they are about 0.5 nm from one another. In some embodiments, the thymine bases or other bases are in proximity when they are about 0.25 nm from one another. In some embodiments, the thymine bases or other bases are in proximity when they are about 0.3 nm to about 0.4 nm from one another. In some embodiments, the thymine bases or other bases are in proximity when they are about 0.35 nm from one another.

In certain embodiments, one or more of the third nucleotide and the fourth nucleotide comprises one or more abasic sites. In certain embodiments, the third nucleotide sequence of the third nucleotide is complementary to the first nucleotide sequence. In certain embodiments, the fourth nucleotide sequence of the fourth nucleotide is complementary to the second nucleotide sequence. In certain embodiments, one or more of the complementarities between the first nucleotide sequence and the third nucleotide sequence and between the second nucleotide sequence and the fourth nucleotide sequence are imperfectly complementary or semi-complementary. In certain embodiments, the first nucleotide sequence and the third nucleotide sequence comprise one or more mismatched bases. In certain embodiments, the second nucleotide sequence and the fourth nucleotide comprise one or more mismatched bases.

In some embodiments, one or more of the first seed nucleotide and the second seed nucleotide is a DNA molecule. In some embodiments, one or more of the first seed nucleotide and the second seed nucleotide is an RNA molecule. In some embodiments, the first seed nucleotide is a DNA molecule, the second seed nucleotide is a DNA molecule, the first seed nucleotide comprises a first thymine base, the second seed nucleotide comprises a second thymine base, the first thymine base and the second thymine base are configured to be brought into proximity by reversible hybridization of the first seed nucleotide sequence to the first nucleotide sequence and the second seed nucleotide sequence to the second nucleotide sequence, and the first thymine base and the second thymine base are configured to dimerize when exposed to ultraviolet light. The thymine bases or other bases are in proximity at any suitable distance. In some embodiments the thymine bases or other bases are in proximity when they are about one base pair distance from one another. In some embodiments, the thymine bases or other bases are in proximity when they are about 0.1 nm to about 1 nm, about 0.1 nm to about 0.75 nm, about 0.1 nm to about 0.5 nm, about 0.25 nm to about 0.5 nm, or about 0.1 nm to about 0.25 nm from one another. In some embodiments, the thymine bases or other bases are in proximity when they are about 0.5 nm from one another. In some embodiments, the thymine bases or other bases are in proximity when they are about 0.25 nm from one another. In some embodiments, the thymine bases or other bases are in proximity when they are about 0.3 nm to about 0.4 nm from one another. In some embodiments, the thymine bases or other bases are in proximity when they are about 0.35 nm from one another.

In certain embodiments, one or more of the first seed and the second seed comprises one or more abasic sites. In certain embodiments, the first seed sequence of the first seed nucleotide is complementary to the first nucleotide sequence. In certain embodiments, the second seed sequence of the second seed nucleotide is complementary to the second nucleotide sequence. In certain embodiments, one or more of the complementarities between the first nucleotide sequence and the first seed sequence and between the second nucleotide sequence and the second seed sequence are imperfectly complementary or semi-complementary. In certain embodiments, the first nucleotide sequence and the first seed sequence comprise one or more mismatched bases. In certain embodiments, the second nucleotide sequence and the second seed sequence comprise one or more mismatched bases.

In some embodiments, the first enzymatic sequence and the second enzymatic sequence are configured to form a Deoxyribozyme (DNAzyme) or a Ribozyme (RNAzyme). In some embodiments, the first enzymatic sequence and the second enzymatic sequence are configured to form a peroxidase-mimicking G-quadruplex Deoxyribozyme (DNAzyme). In some embodiments, the third enzymatic sequence and the fourth enzymatic sequence are configured to form a Deoxyribozyme (DNAzyme) or a Ribozyme (RNAzyme). In some embodiments, the third enzymatic sequence and the fourth enzymatic sequence are configured to form a peroxidase-mimicking G-quadruplex Deoxyribozyme (DNAzyme). In some embodiments, the one or more substrates comprises 2,T-azino-bis(3-ethylbenzothiozoline-6-sulfonic acid) (ABTS) or 3,3′,5,5′-tetramethylbenzidine (TMB), wherein the system further comprises hydrogen peroxide (H₂O₂), and wherein the system further comprises hemin.

In at least one aspect, the method is directed towards detecting a nucleic acid target comprising a first target sequence and a second target sequence. In some embodiments, the nucleic acid target is a DNA molecule. In some embodiments, the nucleic acid target is an RNA molecule.

In some embodiments, the system or method is directed towards a method comprising providing a first nucleotide comprising a first nucleotide sequence and a first enzymatic sequence coupled to the first nucleotide sequence; hybridizing the first nucleotide sequence to the first target sequence; providing a second nucleotide comprising a second nucleotide sequence and a second enzymatic sequence coupled to the second nucleotide sequence; hybridizing the second nucleotide sequence to the second target sequence; dimerizing the first nucleotide and the second nucleotide to form an enzymatically active dimer; providing one or more substrates; and detecting one or more of a decrease in amount of the one or more substrates, a decrease in the concentration of the one or more substrates, an increase in amount of one or more products, and an increase in concentration of one or more products. In some embodiments, the enzymatically active dimer is configured to convert one or more substrates into one or more products. In some embodiments, the method further comprises dissociating the enzymatically active dimer from the nucleic acid target; providing a first seed nucleotide comprising a first seed sequence; hybridizing the first seed sequence the first nucleotide sequence; providing a second seed nucleotide comprising a second seed sequence; hybridizing configured to reversibly hybridize to the second nucleotide sequence; and dimerizing the first seed nucleotide and the second seed nucleotide to form a seed dimer.

In some embodiments, the method further comprises dissociating the enzymatically active dimer from the nucleic acid target; providing a third nucleotide comprising a third nucleotide sequence and a third enzymatic sequence coupled to the third nucleotide sequence; hybridizing the third nucleotide sequence to the first nucleotide sequence; providing a fourth nucleotide comprising a fourth nucleotide sequence and a fourth enzymatic sequence coupled to the fourth nucleotide sequence; hybridizing the fourth nucleotide sequence to the second nucleotide sequence; and dimerizing the third nucleotide and the fourth nucleotide to form a second enzymatically active dimer. In some embodiments, the second enzymatically active dimer is configured to convert the one or more substrates into the one or more product.

In some embodiments, the first nucleotide comprises a first nucleotide thymine base. In some embodiments, the second nucleotide comprises a second nucleotide thymine base. In some embodiments, the step of dimerizing the first nucleotide and the second nucleotide comprises bringing the first nucleotide thymine base into proximity with the second nucleotide thymine base, providing an ultraviolet light source, and forming one or more bonds coupling the first nucleotide thymine base and the second nucleotide thymine base. The thymine bases or other bases are in proximity at any suitable distance. In some embodiments the thymine bases or other bases are in proximity when they are about one base pair distance from one another. In some embodiments, the thymine bases or other bases are in proximity when they are about 0.1 nm to about 1 nm, about 0.1 nm to about 0.75 nm, about 0.1 nm to about 0.5 nm, about 0.25 nm to about 0.5 nm, or about 0.1 nm to about 0.25 nm from one another. In some embodiments, the thymine bases or other bases are in proximity when they are about 0.5 nm from one another. In some embodiments, the thymine bases or other bases are in proximity when they are about 0.25 nm from one another. In some embodiments, the thymine bases or other bases are in proximity when they are about 0.3 nm to about 0.4 nm from one another. In some embodiments, the thymine bases or other bases are in proximity when they are about 0.35 nm from one another.

In some embodiments, the third nucleotide comprises a third nucleotide thymine base. In some embodiments, the fourth nucleotide comprises a fourth nucleotide thymine base. In some embodiments, the step of dimerizing the third nucleotide and the fourth nucleotide comprises bringing the third nucleotide thymine base into proximity with the fourth nucleotide thymine base, providing an ultraviolet light source, and forming one or more bonds coupling the third nucleotide thymine base and the fourth nucleotide thymine base. The thymine bases or other bases are in proximity at any suitable distance. In some embodiments the thymine bases or other bases are in proximity when they are about one base pair distance from one another. In some embodiments, the thymine bases or other bases are in proximity when they are about 0.1 nm to about 1 nm, about 0.1 nm to about 0.75 nm, about 0.1 nm to about 0.5 nm, about 0.25 nm to about 0.5 nm, or about 0.1 nm to about 0.25 nm from one another. In some embodiments, the thymine bases or other bases are in proximity when they are about 0.5 nm from one another. In some embodiments, the thymine bases or other bases are in proximity when they are about 0.25 nm from one another. In some embodiments, the thymine bases or other bases are in proximity when they are about 0.3 nm to about 0.4 nm from one another. In some embodiments, the thymine bases or other bases are in proximity when they are about 0.35 nm from one another.

In some embodiments, the first seed nucleotide comprises a first seed thymine base. In some embodiments, the second seed nucleotide comprises a second seed nucleotide thymine base. In some embodiments, the step of dimerizing the first seed nucleotide and the second seed nucleotide comprises bringing the first seed nucleotide thymine base into proximity with the second seed nucleotide thymine base, providing an ultraviolet light source, and forming one or more bonds coupling the first seed nucleotide thymine base and the second seed nucleotide thymine base. The thymine bases or other bases are in proximity at any suitable distance. In some embodiments the thymine bases or other bases are in proximity when they are about one base pair distance from one another. In some embodiments, the thymine bases or other bases are in proximity when they are about 0.1 nm to about 1 nm, about 0.1 nm to about 0.75 nm, about 0.1 nm to about 0.5 nm, about 0.25 nm to about 0.5 nm, or about 0.1 nm to about 0.25 nm from one another. In some embodiments, the thymine bases or other bases are in proximity when they are about 0.5 nm from one another. In some embodiments, the thymine bases or other bases are in proximity when they are about 0.25 nm from one another. In some embodiments, the thymine bases or other bases are in proximity when they are about 0.3 nm to about 0.4 nm from one another. In some embodiments, the thymine bases or other bases are in proximity when they are about 0.35 nm from one another.

In some embodiments, the first enzymatic sequence comprises a first enzymatic thymine base. In some embodiments, the second enzymatic sequence comprises a second enzymatic nucleotide thymine base. In some embodiments, the step of dimerizing the first enzymatic sequence and the second enzymatic sequence comprises bringing the first enzymatic thymine base into proximity with the second enzymatic thymine base, providing an ultraviolet light source, and forming one or more bonds coupling the first enzymatic thymine base and the second enzymatic thymine base. The thymine bases or other bases are in proximity at any suitable distance. In some embodiments the thymine bases or other bases are in proximity when they are about one base pair distance from one another. In some embodiments, the thymine bases or other bases are in proximity when they are about 0.1 nm to about 1 nm, about 0.1 nm to about 0.75 nm, about 0.1 nm to about 0.5 nm, about 0.25 nm to about 0.5 nm, or about 0.1 nm to about 0.25 nm from one another. In some embodiments, the thymine bases or other bases are in proximity when they are about 0.5 nm from one another. In some embodiments, the thymine bases or other bases are in proximity when they are about 0.25 nm from one another. In some embodiments, the thymine bases or other bases are in proximity when they are about 0.3 nm to about 0.4 nm from one another. In some embodiments, the thymine bases or other bases are in proximity when they are about 0.35 nm from one another.

In some embodiments, the step of detecting one or more of the decrease in amount of the one or more substrates, the decrease in the concentration of the one or more substrates, the increase in amount of one or more products, and the increase in concentration of one or more products comprises detecting the increase in the amount of a 2,2′-azino-bis(3-ethylbenzothiozoline-6-sulfonic acid) (ABTS) radical or detecting the increase in the concentration of an ABTS radical. In some embodiments, the step of detecting one or more of the decrease in amount of the one or more substrates, the decrease in the concentration of the one or more substrates, the increase in amount of one or more products, and the increase in concentration of one or more products comprises detecting the increase in the amount of a 3,3′,5,5′-tetramethylbenzidine (TMB) radical or detecting the increase in the concentration of a TMB radical.

In some embodiments, the Deoxyribozyme (DNAzyme) is split between the two enzymatic sequences at a naturally occurring di-thymine sequence. For example, the peroxidase-mimicking G-quadruplex Deoxyribozyme (DNAzyme) comprises a sequence of 5′-TGGGTAGGGCGGGTTGGGA-3′ (SEQ ID NO: 5). It will be readily recognized that the di-thymine sequence (underlined) is a location to split the Deoxyribozyme (DNAzyme) sequence that, upon dimerization, will produce an enzymatically active Deoxyribozyme (DNAzyme).

Any suitable conditions or components may also be included in the system or method. For example, in some embodiments, the reaction conditions of the system will comprise 25 mM HEPES-NH₄OH (pH of about 8.0), 20 mM KCl, 200 mM NaCl, 1% DMSO, 50 nM hemin, 2 mM H₂O₂, and 2 mM ABTS. In some embodiments, the nucleic acids of the system are present at concentrations of about 1 nM to about 250 nM, about 1 nM to about 100 nM, about 100 nM to about 250 nM, about 1 nM to about 50 nM, about 50 nM to about 100 nM, about 100 nM to about 150 nM, about 150 nM to about 200 nM, about 200 nM to about 250 nM, about 50 nM to about 75 nM, about 75 nM to about 100 nM, about 100 nM to about 125 nM, about 125 nM to about 150 nM, about 80 nM, about 90 nM, about 100 nM, about 110 nM, or about 120 nM. In some embodiments, hemin is present in at least 0.5x the concentrations of the first and second nucleotide so that sufficient hemin is present to bind enzymatically active dimers comprising the first nucleotide and the second nucleotide. It will be recognized that in embodiments comprising third and fourth nucleotides that are configured to form a second enzymatically active dimer, the concentration of hemin may be increased to provide sufficient hemin to bind both the first and second enzymatically active dimers.

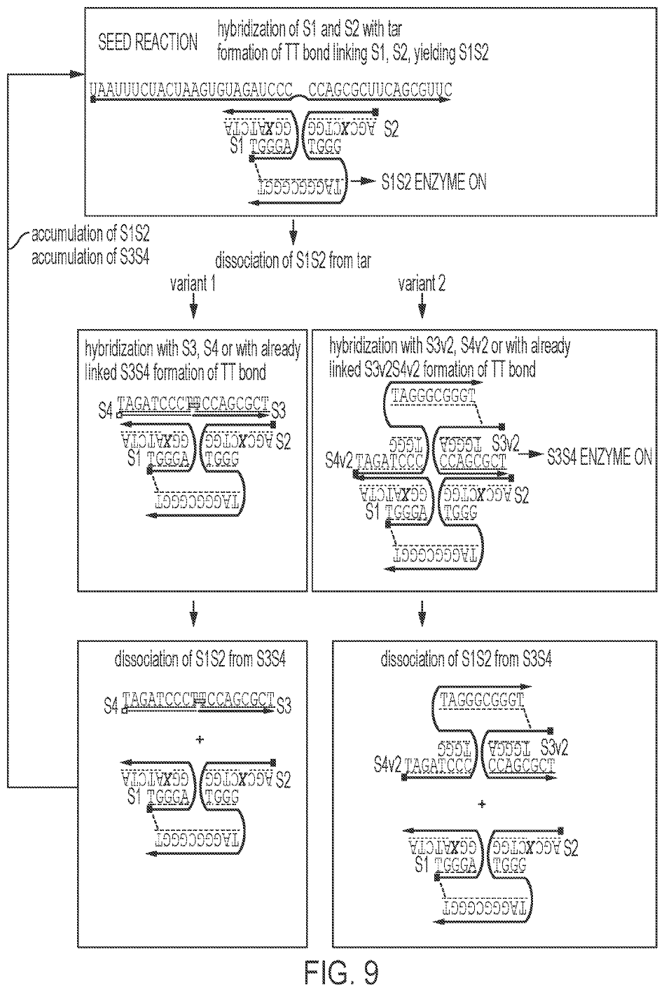

FIG. 9 depicts exemplary embodiments of an aspect of the system or method. FIG. 9 depicts an exemplary embodiment of the system or method to detect the N gene gRNA of the SARS-CoV-2 virus with the sequence:

(SEQ ID NO: 1)
5′-UAAUUUCUACUAAGUGUAGAUCCCCCAGCGCUUCA GCGUUC-3’.

In FIG. 9 two nucleotides are introduced (S1 (5′-TGGGAGGXATCTA-3′ (SEQ ID NO: 6)) and S2 (5′-AGCXCTGGTGGGTAGGGCGGGT-3′ (SEQ ID NO: 7))). The first sequence (5′-GGXATCTA-3′) of the first nucleotide (S1) and the first sequence (5′-AGCXCTGG-3′) of the second nucleotide (S2) are semi-complementary to two separate sequences present on the SARS-CoV-2 virus target. Each of the two nucleotides in the example possess an abasic or mismatch base (X). The second sequence (5′-TGGGA-3′) of the first nucleotide (S1) and the second sequence (5′-TGGGTAGGGCGGGT-3′ (SEQ ID NO: 8)) are enzymatic sequences that are configured to form an enzymatically active Deoxyribozyme (DNAzyme). The 5′ end of one nucleotide (S1) is a thymine base (T). The 3′ end of the other nucleotide (S2) is a thymine base (T). The two nucleotides reversibly hybridize to the target SARS-CoV-2 target. Upon hybridization to the target, the 3′ thymine of S1 and the 5′ thymine of S2 are held in proximity to one another. Exposure of the S1-Tar-S2 complex to an ultraviolet light results in dimerization between the two nucleotide thymines (square and line) dimerizing the two nucleotides (S1S2). The dimerized, enzymatically active S1S2 nucleic acid dissociates from (melts off of) the target SARS-CoV-2. The dissociated target is free to complex another pair of non-dimerized first and second nucleotides, providing signal amplification.

In one embodiment depicted in the left boxes, following dissociation, the nucleotide dimer (S1S2) hybridizes to two semi-complementary nucleotides (S3 and S4). The third nucleotide (S3) comprises a sequence (5′-TCCAGCGCT-3′) is semi-complementary to the first sequence of the second nucleotide and a fourth nucleotide (S4) comprises a sequence (5′-TAGATCCCT-3′) is semi-complementary complementary to the first sequence of firs nucleotide. The 5′ end of the third nucleotide (S3) is a thymine base (T). The 3′ end of the fourth nucleotide (S4) is a thymine base (T). The two nucleotides reversibly hybridize to the enzymatically active dimer (S1S2). Upon hybridization, the 3′ thymine of S4 and the 5′ thymine of S3 are held in proximity to one another. Exposure of the S3-S1S2-S4 complex to an ultraviolet light results in dimerization between the third and fourth nucleotide (S3 and S4) thymines (hatched line) dimerizing the two nucleotides (S3 S4). The dimerized, enzymatically active S1S2 nucleic acid dissociates from (melts off of) the dimerized seed dimer (S3 S4). The dissociated, enzymatically active S1S2 dimer is free to complex another pair of non-dimerized third and fourth nucleotides, providing signal amplification. Following dissociation, the seed dimer (S3 S4) is free to hybridize a pair of non-dimerized first and second nucleotides (S1 and S2). Such dimerization in the presence of an ultraviolet light, provides further dimerized, enzymatically active dimers (S1S2). This also provides signal amplification.

In one embodiment depicted in the right boxes, following dissociation, the nucleotide dimer (S1S2) hybridizes to two semi-complementary nucleotides (S3v2 and S4v2). The third nucleotide (S3v2) comprises a first sequence (5′-CCAGCGCT-3′) that is semi-complementary to the first sequence of the second nucleotide and the fourth nucleotide (S4v2) comprises a first sequence (5′-TAGATCCC-3′) that is semi-complementary complementary to the first sequence of first nucleotide. The third nucleotide (S3v2) comprises a second sequence (5′-TGGGA-3′), and the fourth nucleotide (S4v2) comprises a second sequence (5′-TGGGTAGGGCGGGT-3′ (SEQ ID NO: 8)). The second sequence (5′-TGGGA-3′) of the third nucleotide (S3v2) and the second sequence (5′-TGGGTAGGGCGGGT-3′ (SEQ ID NO: 8)) of the fourth nucleotide (S4v2) are enzymatic sequences that are configured to form an enzymatically active Deoxyribozyme (DNAzyme). The 5′ end of the third nucleotide (S3v2) is a thymine base (T). The 3′ end of the fourth nucleotide (S4v2) is a thymine base (T). The two nucleotides reversibly hybridize to the enzymatically active dimer (S1S2). Upon hybridization, the 3′ thymine of S4v2 and the 5′ thymine of S3v2 are held in proximity to one another. Exposure of the S3v2-S1S2-S4v2 complex to an ultraviolet light results in dimerization between the third and fourth nucleotide (S3v2 and S4v2) thymines (hatched line) dimerizing the two nucleotides (S3v2S4v2). The dimerized, enzymatically active S1S2 nucleic acid dissociates from (melts off of) the dimerized seed dimer (S3v2S4v2). The dissociated, enzymatically active S1S2 dimer is free to complex another pair of non-dimerized third and fourth nucleotides, providing signal amplification. Following dissociation, the seed dimer (S3v2S4v2) is free to hybridize a pair of non-dimerized first and second nucleotides (S1 and S2). Such dimerization in the presence of an ultraviolet light, provides further dimerized, enzymatically active dimers (S1S2). This also provides signal amplification.

In some embodiments depicted in FIG. 9, the enzymatically active dimers are configured to form a peroxidase-mimicking G-quadruplex Deoxyribozyme (DNAzyme) that can enzymatically convert 2,2′-azino-bis(3-ethylbenzothiozoline-6-sulfonic acid) (ABTS) into a radical that is detectable signal (e.g., clear to green). In some embodiments depicted in FIG. 9, the enzymatically active dimers are configured to enzymatically convert 3,3′,5,5′-tetramethylbenzidine (TMB) into a detectable form.

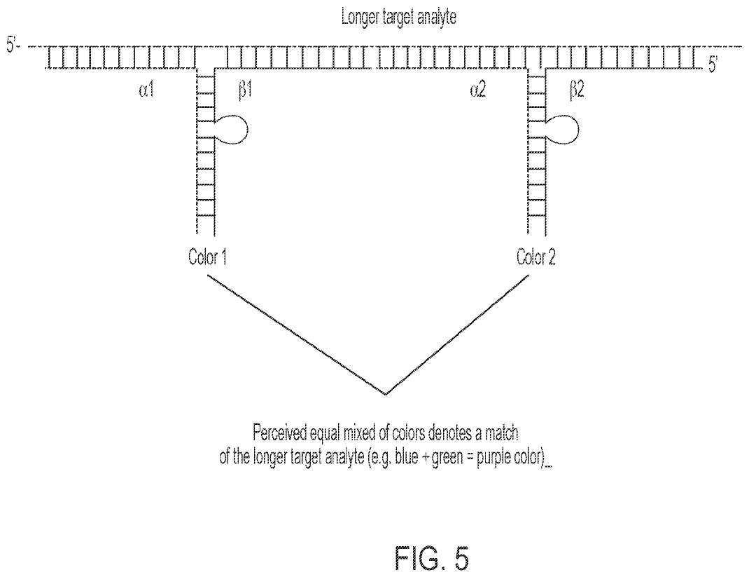

In at least one aspect, one or more of the described systems or methods may be combined with the same or a different system or method described to detect a second analyte. In at least one embodiment, the system or method is directed towards one of the above systems or methods for detecting a target comprising a third target sequence and a fourth target sequence. In some embodiments, the same nucleic acid comprises a plurality of the targets. For example, FIG. 5 depicts an embodiment where two targets are present on a single nucleic acid. In FIG. 5, a longer region of interest is subdivided into two targets that can be detected by the system or method in at least one embodiment. In some embodiments, different nucleic acids comprise each target separately. For example, a first target may be a SARS-CoV-2 nucleotide sequence that would be present on a first nucleic acid and a second target may be an influenza nucleotide sequence that would be present on a second nucleic acid sequence.

In some embodiments, one of the above systems further comprises a fifth nucleotide comprising a fifth nucleotide sequence configured to reversibly hybridize to the third target sequence; a sixth nucleotide comprising a sixth nucleotide sequence configured to reversibly hybridize to the fourth target sequence; a third reporter comprising a third reporter moiety and a third reporter sequence coupled to the third reporter moiety; and a fourth reporter comprising a fourth reporter moiety and a fourth reporter sequence coupled to the fourth reporter moiety. In some embodiments, the third reporter sequence is configured to reversibly hybridize the fifth nucleotide sequence. In some embodiments, the fourth reporter sequence is configured to reversibly hybridize the sixth nucleotide sequence. In some embodiments, the fourth reporter sequence is configured to reversibly hybridize to the third reporter sequence. In some embodiments, the fifth nucleotide and the sixth nucleotide are configured to dimerize. In some embodiments, the third reporter and the fourth reporter are configured to dimerize. In some embodiments, reversible hybridization of the third reporter sequence to the fourth reporter sequence is configured to bring the third reporter moiety and fourth reporter moiety into proximity. In some embodiments, reversible hybridization of one or more of the third reporter sequence to the fifth nucleotide sequence and the fourth reporter sequence to the sixth nucleotide sequence is configured to separate (bring out of proximity) the third reporter moiety and the fourth reporter moiety.

The reporter moieties are in proximity at any suitable distance. In some embodiments the reporter moieties are in proximity when they are about 0.1 nm to about 20 nm, about 0.5 nm to about 15 nm, about 1 nm to about 10 nm, about 2 nm to about 10 nm, about 1 nm, about 2 nm, about 3 nm, about 4 nm, about 5 nm, about 6 nm, about 7 nm, about 8 nm, about 9 nm, about 10 nm, about 11 nm, about 12 nm about 13 nm about 14 nm, or about 15 nm from one another. In some embodiments, both of the reporter moieties are fluorophores, and they are in proximity when they are about 1 nm to about 10 nm, 1 nm, about 2 nm, about 3 nm, about 4 nm, about 5 nm, about 6 nm, about 7 nm, about 8 nm, about 9 nm, or about 10 nm from one another. In some embodiments, one of the reporter moieties is a fluorophore and another reporter moiety is a quencher, and they are in proximity when they are about 1 nm to about 10 nm, about 2 nm to about 10 nm, about 1 nm, about 2 nm, about 3 nm, about 4 nm, about 5 nm, about 6 nm, about 7 nm, about 8 nm, about 9 nm, or about 10 nm from one another.

In some embodiments, one of the above methods further comprises providing a fifth nucleotide comprising a fifth nucleotide sequence; hybridizing the fifth nucleotide sequence to the third target sequence; providing a sixth nucleotide comprising a sixth nucleotide sequence; hybridizing the sixth nucleotide sequence to the fourth target sequence; dimerizing the fifth nucleotide and the sixth nucleotide to form a second dimer; dissociating the second dimer from the target; providing a second reporter complex comprising a third reporter comprising a third reporter moiety and a third reporter sequence coupled to the third reporter moiety, and a fourth reporter comprising a fourth reporter moiety and a fourth reporter sequence coupled to the fourth reporter moiety; dissociating the third reporter sequence from the fourth reporter sequence; hybridizing the third reporter sequence to the fifth nucleotide sequence; hybridizing the fourth reporter sequence to the to the sixth nucleotide; and detecting a change in a second signal produced by one or more of the third reporter moiety and the third reporter moiety. In some embodiments, the fourth reporter sequence is hybridized to the third reporter sequence. In some embodiments, the method further comprises dimerizing the third reporter and the fourth reporter to form a second reporter dimer. In some embodiments, the method further comprises dissociating the second dimer from the second reporter dimer.

In some embodiments, one of the above systems further comprises a fifth nucleotide comprising a fifth nucleotide sequence configured to reversibly hybridize to the third target sequence; a sixth nucleotide comprising a sixth nucleotide sequence configured to reversibly hybridize to the fourth target sequence; a third probe comprising a fifth probe sequence and a sixth probe sequence; a fourth probe comprising a seventh probe sequence and an eighth probe sequence; and a second reporter comprising a third reporter sequence configured to reversibly hybridize the sixth probe sequence, a fourth reporter sequence coupled to the third reporter sequence, a third reporter moiety coupled to third reporter sequence, and a fourth reporter moiety coupled to the fourth reporter sequence. In some embodiments, the fifth probe sequence is configured to reversibly hybridize the fifth nucleotide sequence. In some embodiments, the seventh probe sequence is configured to reversibly hybridize the sixth nucleotide sequence. In some embodiments, the fourth reporter sequence is configured to reversibly hybridize the eighth probe sequence. In some embodiments, the fourth reporter sequence is configured to reversibly hybridize to the third reporter sequence. In some embodiments, the fifth nucleotide and the sixth nucleotide are configured to dimerize. In some embodiments, reversible hybridization of the third reporter sequence to the fourth reporter sequence is configured to bring the third reporter moiety and fourth reporter moiety into proximity. In some embodiments, reversible hybridization of one or more of the third reporter sequence to the sixth probe sequence and the fourth reporter sequence to the eighth probe sequence is configured to separate (bring out of proximity) the third reporter moiety and the fourth reporter moiety.

The reporter moieties are in proximity at any suitable distance. In some embodiments the reporter moieties are in proximity when they are about 0.1 nm to about 20 nm, about 0.5 nm to about 15 nm, about 1 nm to about 10 nm, about 2 nm to about 10 nm, about 1 nm, about 2 nm, about 3 nm, about 4 nm, about 5 nm, about 6 nm, about 7 nm, about 8 nm, about 9 nm, about 10 nm, about 11 nm, about 12 nm about 13 nm about 14 nm, or about 15 nm from one another. In some embodiments, both of the reporter moieties are fluorophores, and they are in proximity when they are about 1 nm to about 10 nm, 1 nm, about 2 nm, about 3 nm, about 4 nm, about 5 nm, about 6 nm, about 7 nm, about 8 nm, about 9 nm, or about 10 nm from one another. In some embodiments, one of the reporter moieties is a fluorophore and another reporter moiety is a quencher, and they are in proximity when they are about 1 nm to about 10 nm, about 2 nm to about 10 nm, about 1 nm, about 2 nm, about 3 nm, about 4 nm, about 5 nm, about 6 nm, about 7 nm, about 8 nm, about 9 nm, or about 10 nm from one another.

In some embodiments, one of the above methods further comprises providing a fifth nucleotide comprising a fifth nucleotide sequence; hybridizing the fifth nucleotide sequence to the third target sequence; providing a sixth nucleotide comprising a sixth nucleotide sequence; hybridizing the sixth nucleotide sequence to the fourth target sequence; dimerizing the fifth nucleotide and sixth nucleotide to form a second dimer; dissociating the second dimer from the target; providing a third probe comprising a fifth probe sequence and a sixth probe sequence; hybridizing the fifth probe sequence to the fifth nucleotide sequence; providing a fourth probe comprising a seventh probe sequence and an eighth probe sequence; hybridizing the seventh probe sequence to the sixth nucleotide sequence; providing a second reporter comprising a third reporter sequence, a fourth reporter sequence coupled to the third reporter sequence, a third reporter moiety coupled to third reporter sequence, and a fourth reporter moiety coupled to the fourth reporter sequence; dissociating the third reporter sequence from the fourth reporter sequence; hybridizing the third reporter sequence to the sixth probe sequence; hybridizing the fourth reporter sequence to the eighth probe sequence; and detecting a change in a second signal produced by one or more of the third reporter moiety and the fourth reporter moiety. In some embodiments, the fourth reporter sequence is hybridized to the third reporter sequence. In some embodiments, the method further comprises dimerizing the third probe and the fourth probe to form a second probe dimer.

In some embodiments, one of the above systems further comprises a fifth nucleotide comprising a fifth nucleotide sequence and a third enzymatic sequence coupled to the fifth nucleotide sequence; a sixth nucleotide comprising a sixth nucleotide sequence and a fourth enzymatic sequence coupled to the sixth nucleotide sequence; and one or more second substrates. In some embodiments, the fifth nucleotide sequence is configured to reversibly hybridize to the third target sequence. In some embodiments, the sixth nucleotide sequence is configured to reversibly hybridize to the fourth target sequence. In some embodiments, the fifth nucleotide and the sixth nucleotide are configured to dimerize. In some embodiments, the dimerized fifth nucleotide and sixth nucleotide is configured to convert the one or more second substrates into one or more second products. In some embodiments, the system further comprises a seventh nucleotide comprising a seventh nucleotide sequence and a fifth enzymatic sequence coupled to the seventh nucleotide sequence; and an eighth nucleotide comprising an eighth nucleotide sequence and a sixth enzymatic sequence coupled to the eighth nucleotide sequence. In some embodiments, the seventh nucleotide sequence is configured to reversibly hybridize to the fifth nucleotide sequence. In some embodiments, the eighth nucleotide sequence is configured to reversibly hybridize to the sixth nucleotide sequence. In some embodiments, the seventh nucleotide and the eighth nucleotide are configured to dimerize. In some embodiments, the dimerized seventh nucleotide and eighth nucleotide is configured to convert the one or more second substrates into one or more second products. In some embodiments, the system further comprises a third seed nucleotide comprising a third seed sequence configured to reversibly hybridize to the fifth nucleotide sequence; and a fourth seed nucleotide comprising a fourth seed sequence configured to reversibly hybridize to the sixth nucleotide sequence. In some embodiments, the third seed nucleotide and the fourth seed nucleotide are configured to dimerize.

In some embodiments, one of the above methods further comprises providing a fifth nucleotide comprising a fifth nucleotide sequence and a third enzymatic sequence coupled to the fifth nucleotide sequence; hybridizing the fifth nucleotide sequence to the third target sequence; providing a sixth nucleotide comprising a sixth nucleotide sequence and a fourth enzymatic sequence coupled to the sixth nucleotide sequence; hybridizing the sixth nucleotide sequence to the fourth target sequence; dimerizing the fifth nucleotide and the sixth nucleotide to form a third enzymatically active dimer; providing the one or more second substrates; and detecting one or more of a decrease in amount of the one or more second substrates, a decrease in the concentration of the one or more second substrates, an increase in amount of the one or more second products, and an increase in concentration of the one or more second products. In some embodiments, the third enzymatically active dimer is configured to convert one or more second substrates into one or more second products. In some embodiments, the method further comprises dissociating the third enzymatically active dimer from the target; providing a third seed nucleotide comprising a third seed sequence; hybridizing the third seed sequence to the fifth nucleotide sequence; providing a fourth seed nucleotide comprising a fourth seed sequence; hybridizing the fourth seed sequence to the sixth nucleotide sequence; and dimerizing the third seed nucleotide and the fourth seed nucleotide to form a second seed dimer. In some embodiments, the method further comprises dissociating the third enzymatically active dimer from the target; providing a seventh nucleotide comprising a seventh nucleotide sequence and a fifth enzymatic sequence coupled to the seventh nucleotide sequence; hybridizing the seventh nucleotide sequence to the fifth nucleotide sequence; providing an eighth nucleotide comprising an eighth nucleotide sequence and a sixth enzymatic sequence coupled to the eighth nucleotide sequence; hybridizing the eighth nucleotide sequence to the sixth nucleotide sequence; and dimerizing the seventh nucleotide and the eighth nucleotide to form a fourth enzymatically active dimer. In some embodiments, the fourth enzymatically active dimer is configured to convert the one or more second substrates into the one or more second products.

In some embodiments, a first and a second analyte may be different targets present on the same molecule. For example, the first analyte may be a first target sequence on a nucleic acid and a second analyte may be a second target sequence on the same nucleic acid. As a further example, the first analyte may be a portion of a protein (e.g., an enzymatic portion of a protein) and the second analyte may be another portion of the protein (e.g., a structural portion of the protein). It will be readily understood that by targeting target sequences on the same nucleic acid will permit detection of longer sequences. In some embodiments, the detection is done separately (e.g., each analyte of the same molecule is detected in a separate reservoir). In some embodiments, the detection is done together (e.g., each analyte of the same molecule is detected in the same reservoir). In some embodiments, the detection is done both separately and together (e.g., two or more analytes of the same molecule are detected in the same reservoir and one or more different analytes of the same molecule are detected in a different reservoir).

In some embodiments, different analytes of the same molecule may overlap. For example, a first sequence target of a molecule may share its terminal sequence with the incipient sequence of a second target of the molecule. In some embodiments, the overlap among two or more analytes may provide the system to detect analytes of a standard length (e.g., of the same or about the same length). It will be readily understood that overlap may provide stability and enhanced confidence in the readout of the system or method.

In some embodiments, the detection of analytes provides the same detectable signal (e.g., the same fluorophore emitting light at the same wavelength). In some embodiments, a positive detection of the molecule is determined by all sensors (e.g., reservoirs) providing a positive (e.g. fluorescent) signal.

In some embodiments, the detection of analytes provides a different detectable signal (e.g., detection of a first analyte provides fluorescence from a first fluorophore at a first wavelength and detection of a second analyte provides fluorescence from a second fluorophore at a second wavelength). In some embodiments, the different detectable signals contrast with one another. In some embodiments, the detection using different detectable signals is provided for in the same reservoir. In some embodiments, detection of the target molecule is determined by a composite signal. FIG. 5 depicts an exemplary system where detection of a target molecule is determined by a composite signal. The target molecule comprises two separate analyte sequences. The system or method provides for detection of the first analyte sequence by producing a first signal (“Color 1”). Upon detection of the first analyte sequence and only the first analyte sequence, the Color 1 signal (e.g., blue) is produced indicating only the first analyte sequence is present. The system or method further provides for detection of the second analyte sequence by producing a second signal (“Color 2”). Upon detection of the second analyte sequence and only the second analyte sequence, the Color 2 signal (e.g., green) is produced indicating only the second analyte sequence is present. Upon detection of both the first analyte sequence and the second analyte sequence in the same sample, both the Color 1 and the Color 2 signals are produced. The composite of the Color 1 and Color 2 signals (e.g., purple) provides an indication that the complete target molecule is present.

In some embodiments, the system or method provides for detection of multiple analytes. In some embodiments, the detection of multiple analytes is provided separately in a single form-factor (e.g., in separate reservoirs). In some embodiments, the detection of multiple analytes is provided in together in a single form-factor (e.g., in a single reservoir).

In some embodiments, the second analyte is of a the same or a similar molecular type as the first analyte. For instance, in embodiments where the first analyte is a nucleic acid (e.g., a DNA or an RNA), the second analyte may also be a nucleic acid (e.g., a second DNA or RNA). As a further example, the first analyte may be a virus protein and the second analyte may be antibodies associated with infection by the virus. In some embodiments, the first and second analyte may be the same analyte (e.g., the same DNA comprising the same DNA sequence). In such embodiments, the system or method may provide for redundant detection of the same analyte by separate sensors. It will be readily understood that such redundant, independent detection of the same analyte provides an increased confidence in detections of the analyte. In some embodiments, the second analyte is of a different molecular type as the first analyte. For instance, in embodiments where the first analyte is a nucleic acid, the second analyte may be a protein, polypeptide, or an oligonucleotide.

In some embodiments, the system or method detects an analyte associated with an infectious agent (e.g., a virus) and an analyte associated with an immune response to the infectious agent (e.g., an antibody). It will be understood that the detecting the combination of an infectious agent analyte and an immune response analyte may be of critical importance in monitoring how quickly and where a vector-borne disease is spreading and correlating such information with the rate at which a population is able to become immune against it. While previously available detection systems (e.g., antibody detections) permit reactive responses to infectious agents, the instant system or method provides an advantage in providing proactive approaches to dealing with infectious diseases by providing both immune response detection and infectious agent detection (e.g., detection of a virus itself from a person's saliva, an object's surfaces, and/or the environment)

In at least one aspect, the system or method disclosed herein is directed towards determining one or more of the concentrations of one or more cations, anions, and salts and the amount of one or more cations, anions, and salts of a composition comprising the nucleic acid target. The phosphate backbone of a nucleic acid carries a negative charge. In hybridization of two strands of nucleic acid, the negative charges of the two phosphate backbones repel one another. However, in the presence of appropriate electrolytes (e.g., cations carrying positive charges), the negative charge of the phosphate backbone is partially or fully neutralized. This neutralization reduces the energy required to bring two nucleic acid strands together to become hybridized. In some embodiments, the system or method makes use of this charge profile to detect electrolyte levels in a sample. In some embodiments, the system or method is directed towards detecting electrolyte levels as a heath biomarker (e.g., the system or method may approximate whole-body hydration levels during rest or exertion).

In some embodiments, the system or method will detect electrolyte levels in a sample from the body. For example, biofluids such as, but not limited to saliva or sweat may be assessed by the system or method. In some embodiments, the system or method is configured to ensure that a sample diffuses into reservoirs rapidly. In some embodiments, the volume of the sample may be any volume. In some embodiments, the volume of the sample is a volume that could be produced (e.g., sweated) rapidly after the start of exertion (e.g., exercise). In some embodiments, the volume of the sample is between about 0.1 μl and about 5 μl, between about 0.1 μl and about 1 μl, between about 1 μl and about 2.5 μl, between about 2.5 μl and about 5 μl, about 1 μl, about 2 μl, about 3 μl, about 4 μl, or about 5 μl, or the volume of the sample is in a range bound by any two values disclosed here. In some embodiments, the system or method may further comprise iontophoretic methods to induce sample production. For example, iontophoretic methods may be used to produce sweat even when a subject is at rest.

In some embodiments, the system or method will be configured to assess the concentration of multiple cations, anions, and/or salts from a single sample. For example, the system may comprise a device including one or more reservoir, wherein each reservoir contains a biosensor system configured to determine the concentration of a specific electrolyte target (e.g., Na⁺, K^{+t}, etc.). In some embodiments, the system comprises a reservoir configured to determine the total ionic strength of multiple electrolytes (e.g., most or all species). In some embodiments, the system includes one or more reservoirs to detect multiple electrolytes (e.g., a reservoir to detect Na⁺ and K^{+t}) and one or more reservoirs to detect specific electrolytes (e.g., a reservoir to detect Na⁺). In some embodiments, the system may further comprise a separate reservoir comprising a sensor system to serve as a control. For example, where saliva is analyzed by the system or method, a positive control may be provided by a reservoir configured to detect a protein found in saliva. In some embodiments, one or more of the reservoirs may be protected by a porous membrane.

In some embodiments, the electrolyte target may be a specific electrolyte that is preferentially extracted from a sample. In some embodiments, extraction of a specific electrolyte may be accomplished by protecting a reservoir with an ion-selective membrane. Such a membrane would separate a sensor from electrolytes not being targeted but present in a sample. Such approaches are known in the art. See Barboiu, Mihail, and Arnaud Gilles. “From natural to bioassisted and biomimetic artificial water channel systems.” Accounts of chemical research 46.12 (2013): 2814-2823; Langecker, Martin, et al. “Synthetic lipid membrane channels formed by designed DNA nanostructures.” Science 338.6109 (2012): 932-936. In one embodiment, extraction of a specific electrolyte may be accomplished by protecting a reservoir with a membrane incorporating one or more ion channels (e.g., channels for K^{+t}, Na⁺, etc.). Such a membrane would permit a sensor to analyze the total ionic strength of a sample. By placing unequal amounts of different channel types, the transient ion stoichiometry may be modified inside the reservoir with respect to the ion stoichiometry in a sample outside the reservoir. In such embodiments, total ionic strength values that correspond more closely with relevant health conditions may be assessed.

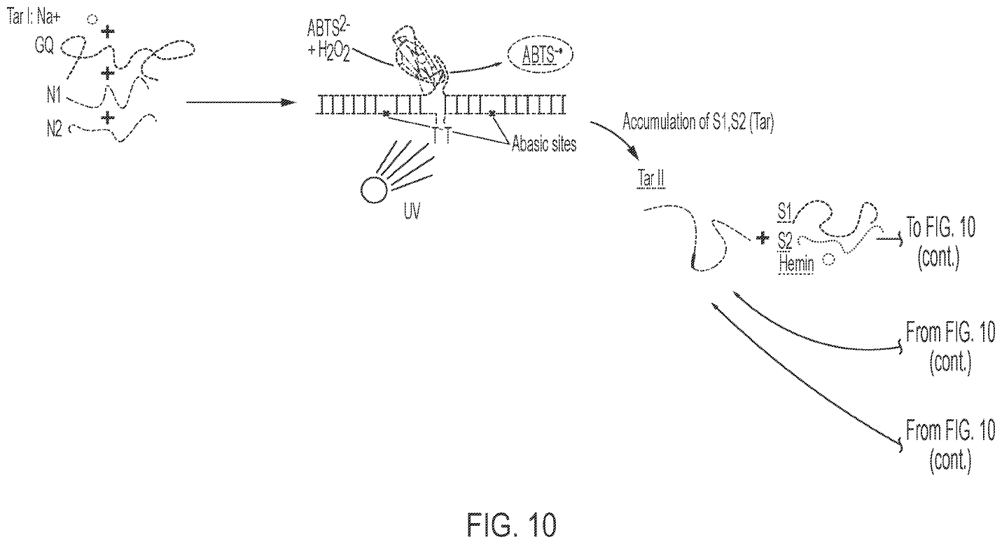

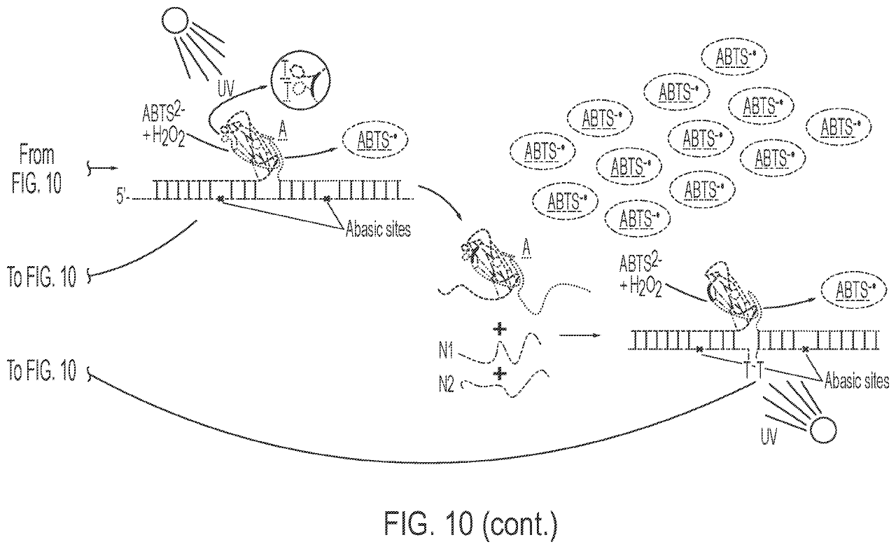

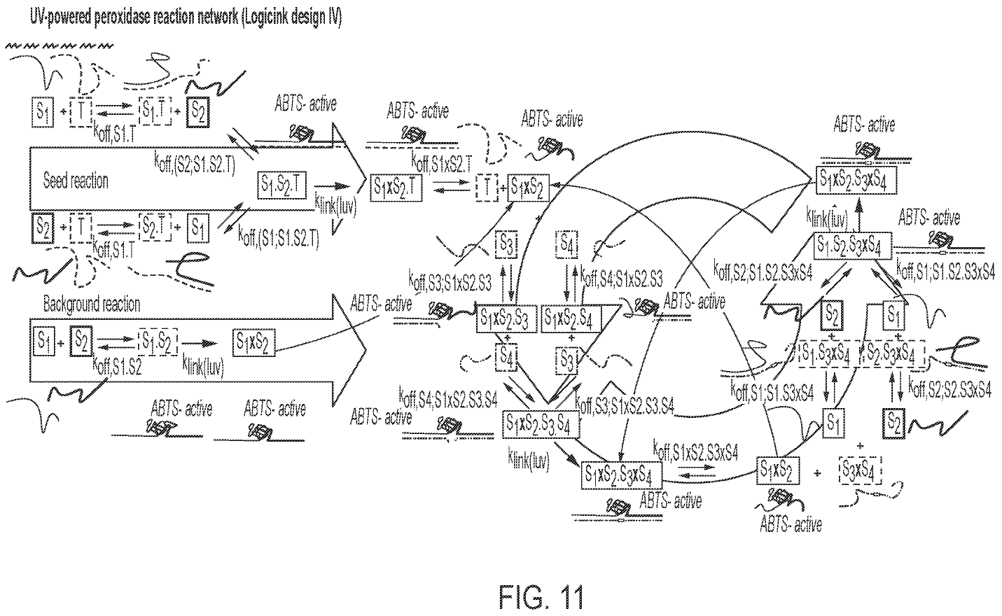

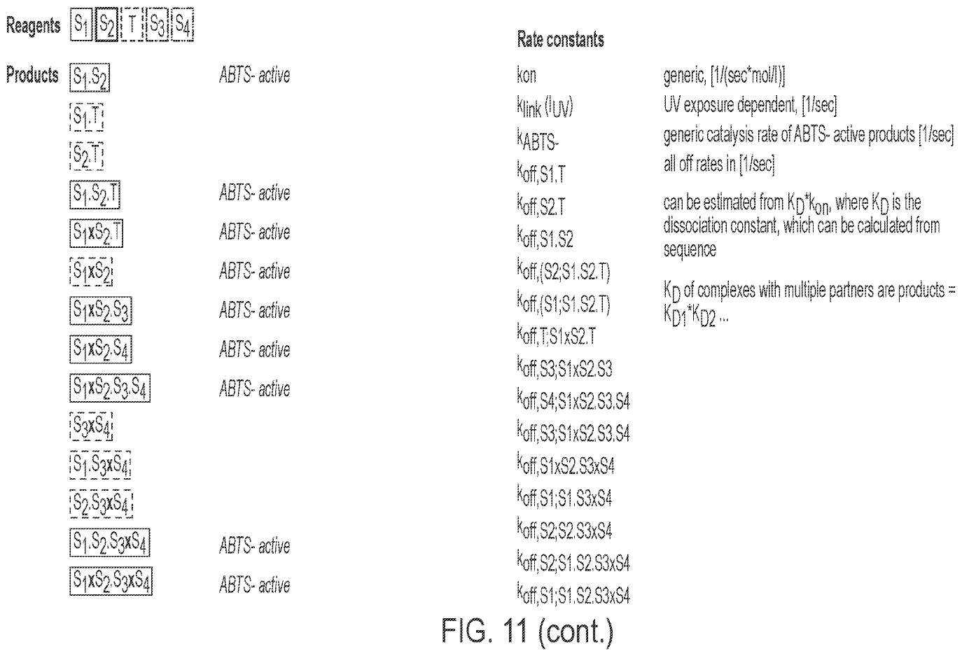

In at least one embodiment, the system or method is based on a Deoxyribozyme (DNAzyme) or Ribozyme (RNAzyme). In some embodiments, the Deoxyribozyme (DNAzyme) is a peroxidase-mimicking G-quadruplex Deoxyribozyme (DNAzyme) that catalyzes generation of a colorimetric signal. Any suitable Deoxyribozyme (DNAzyme) or Ribozyme (RNAzyme) that is capable of producing a detectable signal may be used. The system or method may include any cofactors (e.g., hemin). In some embodiments, the Deoxyribozyme (DNAzyme) or Ribozyme (RNAzyme) is split at a site that can be dimerized (e.g., at two neighboring thymines). For example, FIG. 10 depicts an exemplary embodiment. In the depicted embodiment, the split site is between two adjacent thymines. These thymines are dimerizable in the presence of ultraviolet light. In some embodiments, the Deoxyribozyme (DNAzyme) activity may be boosted by adding a 3′ terminal adenine base. In some embodiments, the system or method provides for exponential signal amplification.

In some embodiments, the system or method is directed towards a system comprising a nucleic acid target comprising a first target sequence and a second target sequence; a first nucleotide comprising a first nucleotide sequence and a first enzymatic sequence coupled to the first nucleotide sequence; a second nucleotide comprising a second nucleotide sequence and a second enzymatic sequence coupled to the second nucleotide sequence; and one or more substrates. In some embodiments, the first nucleotide sequence is configured to reversibly hybridize to the first target sequence when a sufficient concentration of one or more electrolytes are present. In some embodiments, the second nucleotide sequence is configured to reversibly hybridize to the second target sequence when a sufficient concentration of one or more electrolytes are present. In some embodiments, the first nucleotide and the second nucleotide are configured to dimerize. In some embodiments, the dimerized first nucleotide and second nucleotide is configured to convert the one or more substrates into one or more products. In some embodiments, the system further comprises a first seed nucleotide comprising a first seed sequence configured to reversibly hybridize to the first nucleotide sequence; and a second seed nucleotide comprising a second seed sequence configured to reversibly hybridize to the second nucleotide sequence. In some embodiments, the first seed nucleotide and the second seed nucleotide are configured to dimerize.

In some embodiments, the system further comprises a third nucleotide comprising a third nucleotide sequence and a third enzymatic sequence coupled to the third nucleotide sequence; and a fourth nucleotide comprising a fourth nucleotide sequence and a fourth enzymatic sequence coupled to the fourth nucleotide sequence. In some embodiments, the third nucleotide sequence is configured to reversibly hybridize to the first nucleotide sequence wherein the fourth nucleotide sequence is configured to reversibly hybridize to the second nucleotide sequence. In some embodiments, the third nucleotide and the fourth nucleotide are configured to dimerize. In some embodiments, the dimerized third nucleotide and fourth nucleotide is configured to convert the one or more substrates into one or more products.

In some embodiments, one or more of the nucleic acid target, the first nucleotide, the second nucleotide, the third nucleotide, and the fourth nucleotide is a DNA molecule. In some embodiments, one or more of the nucleic acid target, the first nucleotide, the second nucleotide, the third nucleotide, and the fourth nucleotide is an RNA molecule.

In some embodiments, the first nucleotide is a DNA molecule, the second nucleotide is a DNA molecule, the first nucleotide comprises a first thymine base, the second nucleotide comprises a second thymine base, the first thymine base and the second thymine base are configured when a sufficient concentration of one or more electrolytes are present to be brought into proximity by reversible hybridization of the first nucleotide sequence to the first target sequence and the second nucleotide sequence to the second target sequence, and the first thymine base and the second thymine base are configured to dimerize when exposed to ultraviolet light. The thymine bases or other bases are in proximity at any suitable distance. In some embodiments the thymine bases or other bases are in proximity when they are about one base pair distance from one another. In some embodiments, the thymine bases or other bases are in proximity when they are about 0.1 nm to about 1 nm, about 0.1 nm to about 0.75 nm, about 0.1 nm to about 0.5 nm, about 0.25 nm to about 0.5 nm, or about 0.1 nm to about 0.25 nm from one another. In some embodiments, the thymine bases or other bases are in proximity when they are about 0.5 nm from one another. In some embodiments, the thymine bases or other bases are in proximity when they are about 0.25 nm from one another. In some embodiments, the thymine bases or other bases are in proximity when they are about 0.3 nm to about 0.4 nm from one another. In some embodiments, the thymine bases or other bases are in proximity when they are about 0.35 nm from one another.

In certain embodiments, the first nucleotide sequence of the first nucleotide is complementary to the first target sequence. In certain embodiments, the second nucleotide sequence of the second nucleotide is complementary to the second target sequence. In certain embodiments, one or more of the complementarities between the first nucleotide sequence and the first target sequence and between the second nucleotide sequence and the second target sequence are imperfectly complementary or semi-complementary. Methods of producing imperfect or semi-complementary sequences include, but are not limited to, including abasic sites and mismatches in the sequences. Sequences may comprise both one or more abasic sites and one or more mismatches to control for the complementarity of the sequence.

In certain embodiments, one or more of the first nucleotide and the second nucleotide comprises one or more abasic sites. An abasic site may also be referred to as an apurinic or apyrimidinic site. At an abasic site there is neither a purine nor a pyrimidine base, though the phosphate backbone of the RNA or DNA is still present. It is understood that by introducing an abasic site at, e.g., the 5′ end of a nucleotide hybridization between the nucleotide and another nucleic acid will be destabilized. Such destabilization encourages dissociation between two reversibly hybridizable nucleic acids.

In some embodiments, the third nucleotide is a DNA molecule, the fourth nucleotide is a DNA molecule, the third nucleotide comprises a first thymine base, the fourth nucleotide comprises a second thymine base, the first thymine base and the second thymine base are configured to be brought into proximity by reversible hybridization of the third nucleotide sequence to the first nucleotide sequence and the fourth nucleotide sequence to the second nucleotide sequence when a sufficient concentration of one or more electrolytes are present, and the first thymine base and the second thymine base are configured to dimerize when exposed to ultraviolet light. The thymine bases or other bases are in proximity at any suitable distance. In some embodiments the thymine bases or other bases are in proximity when they are about one base pair distance from one another. In some embodiments, the thymine bases or other bases are in proximity when they are about 0.1 nm to about 1 nm, about 0.1 nm to about 0.75 nm, about 0.1 nm to about 0.5 nm, about 0.25 nm to about 0.5 nm, or about 0.1 nm to about 0.25 nm from one another. In some embodiments, the thymine bases or other bases are in proximity when they are about 0.5 nm from one another. In some embodiments, the thymine bases or other bases are in proximity when they are about 0.25 nm from one another. In some embodiments, the thymine bases or other bases are in proximity when they are about 0.3 nm to about 0.4 nm from one another. In some embodiments, the thymine bases or other bases are in proximity when they are about 0.35 nm from one another.

In certain embodiments, one or more of the third nucleotide and the fourth nucleotide comprises one or more abasic sites. In certain embodiments, the third nucleotide sequence of the third nucleotide is complementary to the first nucleotide sequence. In certain embodiments, the fourth nucleotide sequence of the fourth nucleotide is complementary to the second nucleotide sequence. In certain embodiments, one or more of the complementarities between the first nucleotide sequence and the third nucleotide sequence and between the second nucleotide sequence and the fourth nucleotide sequence are imperfectly complementary or semi-complementary. In certain embodiments, the first nucleotide sequence and the third nucleotide sequence comprise one or more mismatched bases. In certain embodiments, the second nucleotide sequence and the fourth nucleotide comprise one or more mismatched bases.

In some embodiments, one or more of the first seed nucleotide and the second seed nucleotide is a DNA molecule. In some embodiments, one or more of the first seed nucleotide and the second seed nucleotide is an RNA molecule. In some embodiments, the first seed nucleotide is a DNA molecule, the second seed nucleotide is a DNA molecule, the first seed nucleotide comprises a first thymine base, the second seed nucleotide comprises a second thymine base, the first thymine base and the second thymine base are configured to be brought into proximity by reversible hybridization of the first seed nucleotide sequence to the first nucleotide sequence and the second seed nucleotide sequence to the second nucleotide sequence when a sufficient concentration of one or more electrolytes are present, and the first thymine base and the second thymine base are configured to dimerize when exposed to ultraviolet light. The thymine bases or other bases are in proximity at any suitable distance. In some embodiments the thymine bases or other bases are in proximity when they are about one base pair distance from one another. In some embodiments, the thymine bases or other bases are in proximity when they are about 0.1 nm to about 1 nm, about 0.1 nm to about 0.75 nm, about 0.1 nm to about 0.5 nm, about 0.25 nm to about 0.5 nm, or about 0.1 nm to about 0.25 nm from one another. In some embodiments, the thymine bases or other bases are in proximity when they are about 0.5 nm from one another. In some embodiments, the thymine bases or other bases are in proximity when they are about 0.25 nm from one another. In some embodiments, the thymine bases or other bases are in proximity when they are about 0.3 nm to about 0.4 nm from one another. In some embodiments, the thymine bases or other bases are in proximity when they are about 0.35 nm from one another.

In certain embodiments, one or more of the first seed and the second seed comprises one or more abasic sites. In certain embodiments, the first seed sequence of the first seed nucleotide is complementary to the first nucleotide sequence. In certain embodiments, the second seed sequence of the second seed nucleotide is complementary to the second nucleotide sequence. In certain embodiments, one or more of the complementarities between the first nucleotide sequence and the first seed sequence and between the second nucleotide sequence and the second seed sequence are imperfectly complementary or semi-complementary. In certain embodiments, the first nucleotide sequence and the first seed sequence comprise one or more mismatched bases. In certain embodiments, the second nucleotide sequence and the second seed sequence comprise one or more mismatched bases.

In some embodiments, the first enzymatic sequence and the second enzymatic sequence are configured to form a Deoxyribozyme (DNAzyme) or a Ribozyme (RNAzyme). In some embodiments, the first enzymatic sequence and the second enzymatic sequence are configured to form a peroxidase-mimicking G-quadruplex Deoxyribozyme (DNAzyme). In some embodiments, the third enzymatic sequence and the fourth enzymatic sequence are configured to form a Deoxyribozyme (DNAzyme) or a Ribozyme (RNAzyme). In some embodiments, the third enzymatic sequence and the fourth enzymatic sequence are configured to form a peroxidase-mimicking G-quadruplex Deoxyribozyme (DNAzyme). In some embodiments, the one or more substrates comprises 2,T-azino-bis(3-ethylbenzothiozoline-6-sulfonic acid) (ABTS) or 3,3′,5,5′-tetramethylbenzidine (TMB), wherein the system further comprises hydrogen peroxide (H₂O₂), and wherein the system further comprises hemin.

In at least one aspect, the system or method is directed towards a method for determining one or more of the concentrations of one or more cations, anions, and salts and the amount of one or more cations, anions, and salts of a composition.

In some embodiments, the system or method is directed towards a method comprising providing a sample; hybridizing a first nucleotide sequence of a first nucleotide further comprising a first enzymatic sequence coupled to the first nucleotide sequence to the first target sequence; hybridizing a second nucleotide sequence of a second nucleotide further comprising a second enzymatic sequence coupled to the second nucleotide sequence to the second target sequence; dimerizing the first nucleotide and the second nucleotide to form an enzymatically active dimer; providing one or more substrates; and detecting one or more of a decrease in amount of the one or more substrates, a decrease in the concentration of the one or more substrates, an increase in amount of one or more products, and an increase in concentration of one or more products. In some embodiments, the first nucleotide sequence is configured to require a sufficient concentration of one or more electrolytes present to hybridize to the first target sequence. In some embodiments, the second nucleotide sequence is configured to require a sufficient concentration of one or more electrolytes present to hybridize to the second target sequence. In some embodiments, the enzymatically active dimer is configured to convert one or more substrates into one or more products. In some embodiments, the method further comprises dissociating the enzymatically active dimer from the nucleic acid target; providing a first seed nucleotide comprising a first seed sequence; hybridizing the first seed sequence the first nucleotide sequence; providing a second seed nucleotide comprising a second seed sequence; hybridizing configured to reversibly hybridize to the second nucleotide sequence; and dimerizing the first seed nucleotide and the second seed nucleotide to form a seed dimer. In some embodiments, the first seed sequence is configured to require a sufficient concentration of one or more electrolytes present to hybridize to the first nucleotide sequence. In some embodiments, the second seed sequence is configured to require a sufficient concentration of one or more electrolytes present to hybridize to the second nucleotide sequence.

In some embodiments, the method further comprises dissociating the enzymatically active dimer from the nucleic acid target; providing a third nucleotide comprising a third nucleotide sequence and a third enzymatic sequence coupled to the third nucleotide sequence; hybridizing the third nucleotide sequence to the first nucleotide sequence; providing a fourth nucleotide comprising a fourth nucleotide sequence and a fourth enzymatic sequence coupled to the fourth nucleotide sequence; hybridizing the fourth nucleotide sequence to the second nucleotide sequence; and dimerizing the third nucleotide and the fourth nucleotide to form a second enzymatically active dimer. In some embodiments, the second enzymatically active dimer is configured to convert the one or more substrates into the one or more product. In some embodiments, the third nucleotide sequence is configured to require a sufficient concentration of one or more electrolytes present to hybridize to the first nucleotide sequence. In some embodiments, the fourth nucleotide sequence is configured to require a sufficient concentration of one or more electrolytes present to hybridize to the second nucleotide sequence.

In some embodiments, the first nucleotide comprises a first nucleotide thymine base. In some embodiments, the second nucleotide comprises a second nucleotide thymine base. In some embodiments, the step of dimerizing the first nucleotide and the second nucleotide comprises bringing the first nucleotide thymine base into proximity with the second nucleotide thymine base, providing an ultraviolet light source, and forming one or more bonds coupling the first nucleotide thymine base and the second nucleotide thymine base. The thymine bases or other bases are in proximity at any suitable distance. In some embodiments the thymine bases or other bases are in proximity when they are about one base pair distance from one another. In some embodiments, the thymine bases or other bases are in proximity when they are about 0.1 nm to about 1 nm, about 0.1 nm to about 0.75 nm, about 0.1 nm to about 0.5 nm, about 0.25 nm to about 0.5 nm, or about 0.1 nm to about 0.25 nm from one another. In some embodiments, the thymine bases or other bases are in proximity when they are about 0.5 nm from one another. In some embodiments, the thymine bases or other bases are in proximity when they are about 0.25 nm from one another. In some embodiments, the thymine bases or other bases are in proximity when they are about 0.3 nm to about 0.4 nm from one another. In some embodiments, the thymine bases or other bases are in proximity when they are about 0.35 nm from one another.

In some embodiments, the third nucleotide comprises a third nucleotide thymine base. In some embodiments, the fourth nucleotide comprises a fourth nucleotide thymine base. In some embodiments, the step of dimerizing the third nucleotide and the fourth nucleotide comprises bringing the third nucleotide thymine base into proximity with the fourth nucleotide thymine base, providing an ultraviolet light source, and forming one or more bonds coupling the third nucleotide thymine base and the fourth nucleotide thymine base. The thymine bases or other bases are in proximity at any suitable distance. In some embodiments the thymine bases or other bases are in proximity when they are about one base pair distance from one another. In some embodiments, the thymine bases or other bases are in proximity when they are about 0.1 nm to about 1 nm, about 0.1 nm to about 0.75 nm, about 0.1 nm to about 0.5 nm, about 0.25 nm to about 0.5 nm, or about 0.1 nm to about 0.25 nm from one another. In some embodiments, the thymine bases or other bases are in proximity when they are about 0.5 nm from one another. In some embodiments, the thymine bases or other bases are in proximity when they are about 0.25 nm from one another. In some embodiments, the thymine bases or other bases are in proximity when they are about 0.3 nm to about 0.4 nm from one another. In some embodiments, the thymine bases or other bases are in proximity when they are about 0.35 nm from one another.

In some embodiments, the first seed nucleotide comprises a first seed thymine base. In some embodiments, the second seed nucleotide comprises a second seed nucleotide thymine base. In some embodiments, the step of dimerizing the first seed nucleotide and the second seed nucleotide comprises bringing the first seed nucleotide thymine base into proximity with the second seed nucleotide thymine base, providing an ultraviolet light source, and forming one or more bonds coupling the first seed nucleotide thymine base and the second seed nucleotide thymine base. The thymine bases or other bases are in proximity at any suitable distance. In some embodiments the thymine bases or other bases are in proximity when they are about one base pair distance from one another. In some embodiments, the thymine bases or other bases are in proximity when they are about 0.1 nm to about 1 nm, about 0.1 nm to about 0.75 nm, about 0.1 nm to about 0.5 nm, about 0.25 nm to about 0.5 nm, or about 0.1 nm to about 0.25 nm from one another. In some embodiments, the thymine bases or other bases are in proximity when they are about 0.5 nm from one another. In some embodiments, the thymine bases or other bases are in proximity when they are about 0.25 nm from one another. In some embodiments, the thymine bases or other bases are in proximity when they are about 0.3 nm to about 0.4 nm from one another. In some embodiments, the thymine bases or other bases are in proximity when they are about 0.35 nm from one another.

In some embodiments, the first enzymatic sequence comprises a first enzymatic thymine base. In some embodiments, the second enzymatic sequence comprises a second enzymatic nucleotide thymine base. In some embodiments, the step of dimerizing the first enzymatic sequence and the second enzymatic sequence comprises bringing the first enzymatic thymine base into proximity with the second enzymatic thymine base, providing an ultraviolet light source, and forming one or more bonds coupling the first enzymatic thymine base and the second enzymatic thymine base. The thymine bases or other bases are in proximity at any suitable distance. In some embodiments the thymine bases or other bases are in proximity when they are about one base pair distance from one another. In some embodiments, the thymine bases or other bases are in proximity when they are about 0.1 nm to about 1 nm, about 0.1 nm to about 0.75 nm, about 0.1 nm to about 0.5 nm, about 0.25 nm to about 0.5 nm, or about 0.1 nm to about 0.25 nm from one another. In some embodiments, the thymine bases or other bases are in proximity when they are about 0.5 nm from one another. In some embodiments, the thymine bases or other bases are in proximity when they are about 0.25 nm from one another. In some embodiments, the thymine bases or other bases are in proximity when they are about 0.3 nm to about 0.4 nm from one another. In some embodiments, the thymine bases or other bases are in proximity when they are about 0.35 nm from one another.

In some embodiments, the step of detecting one or more of the decrease in amount of the one or more substrates, the decrease in the concentration of the one or more substrates, the increase in amount of one or more products, and the increase in concentration of one or more products comprises detecting the increase in the amount of a 2,2′-azino-bis(3-ethylbenzothiozoline-6-sulfonic acid) (ABTS) radical or detecting the increase in the concentration of an ABTS radical. In some embodiments, the step of detecting one or more of the decrease in amount of the one or more substrates, the decrease in the concentration of the one or more substrates, the increase in amount of one or more products, and the increase in concentration of one or more products comprises detecting the increase in the amount of a 3,3′,5,5′-tetramethylbenzidine (TMB) radical or detecting the increase in the concentration of a TMB radical.

FIG. 10 depicts an exemplary embodiment of an aspect of the system or method.

The system of FIG. 10 is divided in two parts. The initial target in part I is sodium cation (Na⁺). When the cation is present at a sufficient concentration, the first nucleotide (N1) and the second nucleotide (N2) are able to hybridize to a G-quadruplex (GQ), which selectively requires sodium cation, rather than hemin as shown in previous literature. Both N1 and N2 possess terminal thymine bases, and upon hybridization with GQ the thymine bases are brought into proximity with one another. Upon exposure to ultraviolet light the terminal thymine bases dimerize. GQ and the N1N2 dimer dissociate (melt off). Thus, GQ is available to hybridize another N1 and another N2, thereby providing signal amplification.

The N1N2 dimer of part I is the second target. As discussed above in detail, a dimer such as N1N2 can be used to produce a Deoxyribozyme-based (DNAzyme-based) readout as depicted in FIG. 10. Further, signal amplification may be provided by the methods discussed above and in FIGS. 7-9. Modification of these examples to detect and an N1N2 dimer associated with an electrolyte concentration will be readily apparent to those of skill in the art.

In some embodiments, the system or method may be modified so that seeding reactions may be calibrated to target other electrolytes, or other types of molecules. In such embodiments, a nucleic acid dimer may be provided to part II of the system or method to permit a detectable readout.

Use of modified G-quadruplex Deoxyribozymes (DNAzymes) is a powerful generalizable method to target a range of analytes while keeping a common amplification system. Such system commonality is highly beneficial when mass scaling production.

This approach is not mutually exclusive of the extraction methods relying on the use of ion-selective membranes. In some embodiments, a seeding reaction may give further control over the range affecting the sensor output. In some embodiments, the system or method does not require an ion-selective membrane.

In some embodiments, the system or method may provide progressive electrolyte information. For example, a subject exerting themselves (e.g., performing exercise) may provide a sample (e.g., lick a sensor or place a sensor on their skin) at the start of the activity. At a later timepoint, the subject may provide a second sample, and continue exerting themselves. In this manner, the system or method may provide temporal information concerning the hydration and/or electrolyte concentration. Samples may be taken at predetermined time periods. The system or method may be coupled to a computer device (e.g., a mobile phone) to provide reminders to a subject to test their hydration and/or electrolyte levels.

In some embodiments, a single device may provide multiple sensors. In some embodiments, each sensor is protected separately (e.g., by a thin cover with a tab). The protectors may be individually removed, and the sensor may be individually exposed to a sample (e.g., saliva) so as to avoid contaminating the other sensors. A subject may remove the separate protectors periodically while exerting themselves. In some embodiments, the subject is not exerting themselves. In some embodiments, the subject is sedentary people. For example, elderly subjects may use the system or method to monitor approximate hydration levels.

It will be readily understood that any of the systems contemplated in this application may further comprise an ultraviolet light source. In some embodiments, the system of the system or method comprising a module comprising a sensor configured to detect ultraviolet light; a pathway configured to output a comparison comparing the cumulative ultraviolet light detected by the sensor to a predetermined threshold; and a display configured to display a value associated with one or more of the ultraviolet light detected by the sensor, the cumulative ultraviolet light detected by the sensor, and the comparison.

In some embodiments, ultraviolet irradiation may be provided by ambient ultraviolet light (e.g., light from the sun). Any suitable dosage of ultraviolet light is contemplated. In some embodiments, the dosage of ultraviolet light is about 1 joule/cm² to about 100 joule/cm², about 1 joule/cm² to about 50 joule/cm², about 50 joule/cm² to about 100 joule/cm², about 1 joule/cm² to about 25 joule/cm², about 25 joule/cm² to about 50 joule/cm², about 75 joule/cm² to about 100 joule/cm², about 1 joule/cm², about 10 joule/cm², about 20 joule/cm², about 30 joule/cm², about 40 joule/cm², about 50 joule/cm², about 60 joule/cm², about 70 joule/cm², about 80 joule/cm², about 90 joule/cm², or about 100 joule/cm². In at least one embodiment, the dosage of ultraviolet light is about 40 joule/cm². Embodiments using an ultraviolet dose to induce dimerization can be used in to detect multiple analytes.

In some embodiments, signal amplification occurs rapidly. In some embodiments, signal amplification occurs over a period of time. For example, in some embodiments, a sample is provided (e.g., a user licks a device comprising the system) before going to sleep at night. The signal amplification occurs throughout the night, and, upon the user's waking up in the morning, the system or method provides the user with a readily understandable readout regarding the user's status (e.g., they are negative for an analyte associated with an infection agent). In some embodiments, signal amplification occurring over a period of time provides a high signal to noise ratio. It will be understood that a high signal to noise ratio allows a high degree of sensitivity. In some embodiments, the high degree of sensitivity is provided in colorimetric sensors.

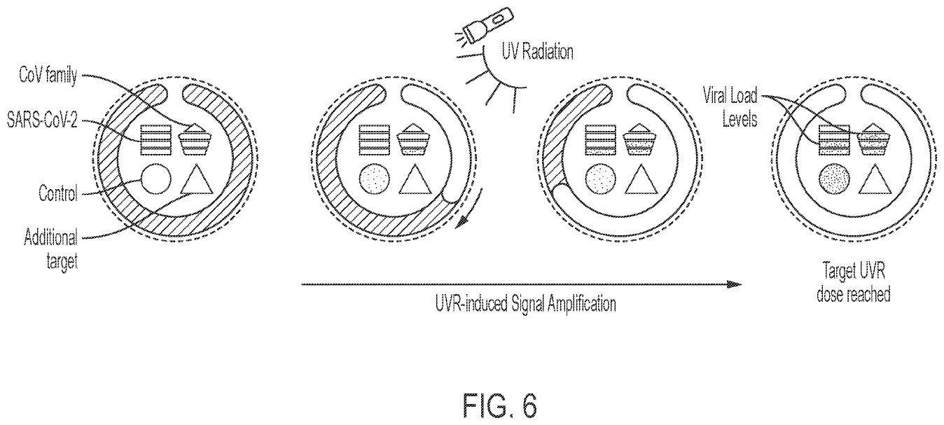

Any of the designs or methods disclosed herein may be in any appropriate form-factor or use-case including, but not limited to, a skin wearable, a sticker on an object's surface, a two-dimensional applique, or a substrate (e.g. a polymeric film substrate). In some embodiments, the form-factor or use-case may further define one or more reservoir protected by a porous membrane. A reservoir may contain a biosensor system focused on a one or more specific analytes, for example a specific RNA or DNA sequence being targeted. For example, FIG. 6 depicts an exemplary form-factor comprising four separate reservoirs. In FIG. 6, the four reservoirs correspond to a biosensor system for detecting an analyte associated with SARS-CoV-2 (i.e., SARS-CoV-2), an analyte associated with virus from the CoV family, an additional analyte target, and a control target. In some embodiments the reservoirs act as homogeneous assays where all biosensor reactions occur concurrently. The reservoirs provide separation among the biosensor systems to avoid cross-reaction effects.

The reservoir may be protected with a lid or seal that can be peeled or partially peeled back. In some embodiments, the lid or seal is replaced after a source (e.g., saliva deposited by a lick) is provided. The form-factor may comprise a control on the borders of one or more of the reservoirs that provides an indication upon the application of a source. For example, the border may change color (e.g., from clear to blue) when presented with a detectable characteristic of the source (e.g., a pH associated with a source such as saliva). Preferably, the indication is instantaneous or near-instantaneous. The indication corresponds to a sufficient amount of source provided to the associated reservoir.

In certain embodiments, the form-factor may comprise a design configured to dimerize one or more pairs by use of an ultraviolet irradiation. In such embodiments, the form-factor may comprise an indicator corresponding to a sufficient exposure to ultraviolet light. For example, an indicator may change color upon exposure to an ultraviolet light source. In certain embodiments, the indicator's color or degree of its color change can provide a quantitative estimate of the sufficiency of the ultraviolet irradiance. For example, FIG. 6 depicts an exemplary form-factor comprising an outer ring that changes color when exposed to an ultraviolet light source. As the ring is exposed to ultraviolet light is changes color from a dark color to a lighter color indicating progressive cumulative exposure to irradiance. A change from the dark color to the light color along the entire ring or some predetermined portion of the ring is associated with sufficient and complete irradiance.

In certain embodiments, the form-factor provides a readily understandable readout of the presence or absence of the analyte. In some embodiments, the readily understandable readout is provided as a color change of one or multiple bars, wherein a color change in no bars is associated with the absence of the analyte of interest, a color change in all bars is associated with a high level of presence of the analyte of interest, and a color change in some but not all bars is associated with an intermediate presence of the analyte of interest. In some embodiments, the associated level of presence of an analyte corresponds to an estimate of a viral load. FIG. 6 depicts an exemplary form-factor comprising semi-quantitative indicators for the determination of an analytes' presence. In FIG. 6, the square indicator and pentagram indicator are semi-quantitative, possessing multiple bars associated with analyte presence, wherein the more bars that undergo a color change from a light color to a dark color correlates to the amount of the analyte (e.g., viral load indicator) present in a sample.

### EXAMPLES

### Example 1

Detection of RNA Targets by Molecular Beacons

RNA targets may be detected using the instant system or method employing one or more molecular beacons.

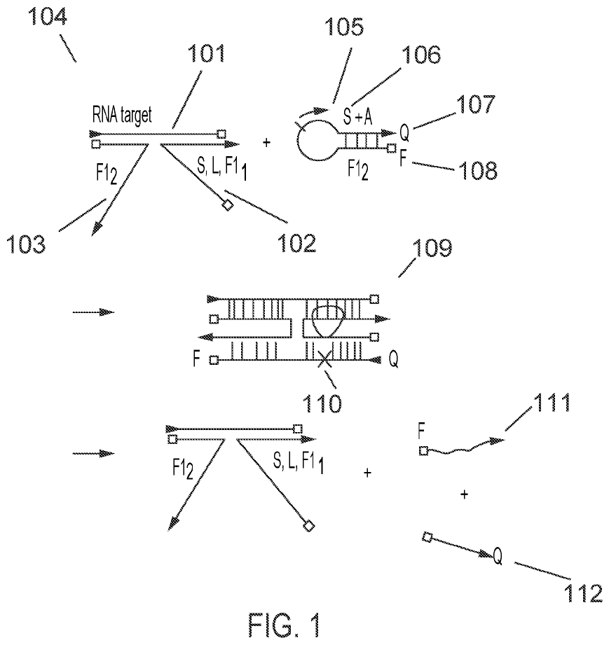

FIG. 1 depicts an RNA target 101. Partially complementary nucleic acids are added. The first partially complementary nucleic acid 102 (F1₁) and the second partially complementary nucleic acid 103 (F1₂), partially hybridize to the RNA target 101. This forms a probe-target complex 104.

A pool of molecular beacons 105 are added to the probe-target complex 104. The molecular beacons 105 each have a spacer 106 (S) and a catalytic coop (C). The molecular beacon 105 has a fluorophore 107 (F) attached at one terminal end and a quenching molecule 108 (Q) at the opposite end that are in proximity when the beacon is free.

The molecular beacon is partially complementary to the sequence of the first partially complementary nucleic acid 102 and the second partially complementary nucleic acid 103 that are not complementary to the RNA target 101. Thus, the molecular beacon 104 can co-hybridize with the bound 102 and 103 probes based on its own partial complementarity. This forms a four-nucleic acid complex 109.

Upon hybridization between the molecular beacon 105 and the probe-target complex 104, the four-nucleic acid complex 109 reveals a cite for enzymatic restriction 109 (X) on the molecular beacon 105. At this enzymatic restriction cite 110, enzymatic cleavage will occur. This leads to separating the molecular beacon end attached to the fluorophore 111 and the end attached to the quencher 112.

Upon release from the partially complementary nucleic acids, the fluorophore end of the molecular beacon 111 and the quencher end of the molecular beacon 112 will diffuse, thereby separating (bringing out of proximity) the fluorophore 107 from the quencher 108 and resulting in a detectable signal.

Because multiple molecular beacons can be processed by a single RNA target, the signal may be amplified in proportion to the number of RNA targets present in a sample.

### Example 2

Detection of SARS-CoV-2 Virus

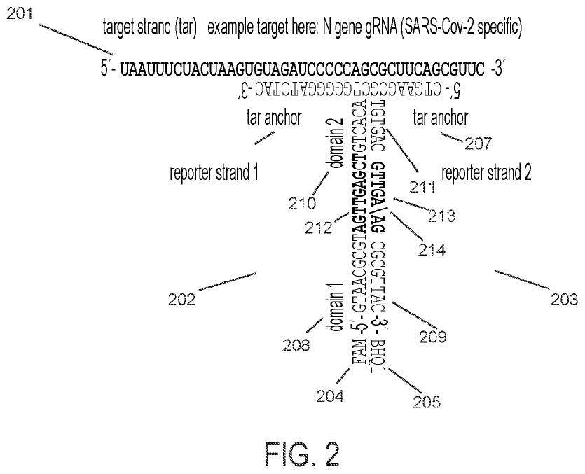

The instant system or method may be used to detect the presence of the SARS-CoV-2 virus as depicted in FIG. 2.

A target nucleic acid 201 from the genomic RNA of the SARS-CoV-2 virus is provided to the indicator. The indicator possesses a sensor comprising reporter strand 1 202 and reporter strand 2 203 in nearly molecular equivalents.

Report strand 1 202 possesses a fluorophore 204 (FAM). Reporter strand 2 203 possesses a quencher 205 (BHQ1).

Report strand 1 and reporter strand 2 have target anchors 206, 207. The target anchors 206, 207 are partially complementary to the target nucleic acid 201. The partially complementary target anchors 206, 207 hybridize with the target sequence 201.

Reporter strand 1 and report strand 2 both have a first domain 208, 209 (domain 1) and a second domain 210, 211 (domain 2). The first domain of reporter strand 1 208 is complementary to the first domain of reporter strand 2 209. These first domains 208, 209 hybridize to one another. The second domain of reporter strand 1 210 is complementary to the second domain of reporter strand 2 211. These second domains 210, 211 hybridize to one another. And so the reporter strands 202, 203 are able to hybridize at their first domains 208, 209 and further at their second domains 210, 211.

Reporter strand 1 and reporter strand 2 both have a non-complementary sequence 212, 213 (shown in bold in FIG. 2). These non-complementary sequences 212, 213 separate the first domain 208, 209 and the second domain 210, 211 of the same reporter strand. These non-complementary sequences 212, 213 do not hybridize to one another.

In the absence of the SARS-CoV-2 virus gRNA target sequence, the two reporter strands will hybridize with one another at the first domains 208, 209 and the second domains 210, 211. This hybridization will bring the quencher 205 into proximity with the fluorophore 204. However, the non-complementary regions 212, 213 will not produce a catalytic loop for cleavage. The resulting quenching the emissions of the fluorophore is a detectable signal indicating the absence of the SARS-CoV-2 virus.

In the presence of the SARS-CoV-2 virus gRNA target sequence 201, the reporter strands 202, 203 will further partially hybridize with the target sequence by the target anchors 206, 207. This additional hybridization will further stabilize the reporter strand 1 202-reporter strand 2 203 hybridization. This will allow for catalytic loop formation at the non-complementary region of reporter strand 2 213.

In the presence of Deoxyribozyme (DNAzyme) and about 1 mM Zn²⁺, restriction occurs at the catalytic loop 214 (“I” in FIG. 2) at the non-complementary region of reporter strand 2 213. The resulting fragments of reporter strand 2 along with the BHQ1 quencher dissociates from the hybrid complex, thereby increasing the detectable fluorescent emission from the fluorophore, FAM. The increase detectable increase of emissions of the fluorophore acts as an indicator of the presence of the SARS-CoV-2 virus.

As will be apparent to one of ordinary skill in the art from a reading of this disclosure, the present disclosure can be embodied in forms other than those specifically disclosed above. The particular embodiments described above are, therefore, to be considered as illustrative and not restrictive. Those skilled in the art will recognize, or be able to ascertain, using no more than routine experimentation, numerous equivalents to the specific embodiments described herein.

All references and publications recited are incorporated by reference.

1. A system of detecting a nucleic acid target comprising a first target sequence and a second target sequence, wherein the system comprises:
a first nucleotide comprising a first nucleotide sequence configured to reversibly hybridize the first target sequence;
a second nucleotide comprising a second nucleotide sequence configured to reversibly hybridize the second target sequence;
wherein the first nucleotide and the second nucleotide are configured to dimerize to form a first dimer upon reversible hybridization of the first nucleotide sequence to the first target sequence and reversible hybridization of the second nucleotide sequence to the second target sequence;
a first reporter comprising a first reporter moiety and a first reporter sequence coupled to the first reporter moiety, wherein the first reporter sequence is configured to reversibly hybridize the first nucleotide sequence; and
a second reporter comprising a second reporter moiety and a second reporter sequence coupled to the second reporter moiety, wherein the second reporter sequence is configured to reversibly hybridize the second nucleotide sequence, and wherein the second reporter sequence is configured to reversibly hybridize the first reporter sequence;
wherein the first reporter and the second reporter are configured to dimerize to form a reporter dimer upon reversible hybridization of the first reporter sequence to the first nucleotide sequence of the first dimer and reversible hybridization of the second reporter sequence to the second nucleotide sequence of the first dimer,
wherein the first reporter moiety is configured to produce a first reporter moiety signal, wherein the second reporter moiety is configured to alter the first reporter moiety signal when the first reporter moiety and the second reporter moiety are in proximity,
wherein reversible hybridization of the first reporter sequence to the second reporter sequence is configured to bring the first reporter moiety and the second reporter moiety into proximity, and
wherein reversible hybridization of one or more of the first reporter sequence to the first nucleotide sequence of the first dimer and the second reporter sequence to the second nucleotide sequence of the first dimer is configured to separate the first reporter moiety and the second reporter moiety.

2. A system of detecting a nucleic acid target comprising a first target sequence and a second target sequence, wherein the system comprises:
a first nucleotide comprising a first nucleotide sequence configured to reversibly hybridize the first target sequence;
a second nucleotide comprising a second nucleotide sequence configured to reversibly hybridize the second target sequence;
wherein the first nucleotide and the second nucleotide are configured to dimerize to form a first dimer upon reversible hybridization of the first nucleotide sequence to the first target sequence and reversible hybridization of the second nucleotide sequence to the second target sequence;
a first probe comprising a first probe sequence and a second probe sequence, wherein the first probe sequence is configured to reversibly hybridize the first nucleotide sequence;
a second probe comprising a third probe sequence and a fourth probe sequence, wherein the third probe sequence is configured to reversibly hybridize the second nucleotide sequence;
wherein the first probe and the second probe are configured to dimerize to form a first probe dimer upon reversible hybridization of the first probe sequence to the first nucleotide sequence of the first dimer and reversible hybridization of the third probe sequence to the second nucleotide sequence of the first dimer; and
a reporter comprising:
a first reporter sequence configured to reversibly hybridize the second probe sequence,
a second reporter sequence coupled to the first reporter sequence, wherein the second reporter sequence is configured to reversibly hybridize the fourth probe sequence, and wherein the second reporter sequence is configured to reversibly hybridize the first reporter sequence,
a first reporter moiety coupled to first reporter sequence, and
a second reporter moiety coupled to the second reporter sequence;
wherein the first reporter moiety is configured to produce a first reporter moiety signal, wherein the second reporter moiety is configured to alter the first reporter moiety signal when the first reporter moiety and the second reporter moiety are in proximity,
wherein reversible hybridization of the first reporter sequence to the second reporter sequence is configured to bring the first reporter moiety and the second reporter moiety into proximity, and
wherein reversible hybridization of one or more of the first reporter sequence to the second probe sequence of the first probe dimer and the second reporter sequence to the fourth probe sequence of the first probe dimer is configured to bring the first reporter moiety and the second reporter moiety out of proximity.

3. A system of detecting a nucleic acid target comprising a first target sequence and a second target sequence, wherein the system comprises:
a first nucleotide comprising a first nucleotide sequence and a first enzymatic sequence coupled to the first nucleotide sequence, wherein the first nucleotide sequence is configured to reversibly hybridize the first target sequence;
a second nucleotide comprising a second nucleotide sequence and a second enzymatic sequence coupled to the second nucleotide sequence, wherein the second nucleotide sequence is configured to reversibly hybridize the second target sequence;
wherein the first nucleotide and the second nucleotide are configured to dimerize to form a first enzymatically active dimer upon reversible hybridization of the first nucleotide sequence to the first target sequence and reversible hybridization of the second nucleotide sequence to the second target sequence; and
one or more first substrates;
wherein the first enzymatically active dimer is configured to convert the one or more first substrates into one or more first products.

4. The system of claim 3, further comprising:
a third nucleotide comprising a third nucleotide sequence and a third enzymatic sequence coupled to the third nucleotide sequence, wherein the third nucleotide sequence is configured to reversibly hybridize the first nucleotide sequence; and
a fourth nucleotide comprising a fourth nucleotide sequence and a fourth enzymatic sequence coupled to the fourth nucleotide sequence, wherein the fourth nucleotide sequence is configured to reversibly hybridize the second nucleotide sequence;
wherein the third nucleotide and the fourth nucleotide are configured to dimerize to form a second enzymatically active dimer upon reversible hybridization of the third nucleotide sequence to the first nucleotide sequence of the first enzymatically active dimer and the fourth nucleotide sequence to the second nucleotide sequence of the first enzymatically active dimer, and
wherein the second enzymatically active dimer is configured to convert the one or more first substrates into one or more first products.

5. The system of any of claims 3 and 4, further comprising:
a first seed nucleotide comprising a first seed sequence configured to reversibly hybridize the first nucleotide sequence; and
a second seed nucleotide comprising a second seed sequence configured to reversibly hybridize the second nucleotide sequence;
wherein the first seed nucleotide and the second seed nucleotide are configured to dimerize to form a first seed dimer upon reversible hybridization of the first seed nucleotide to the first nucleotide sequence of the first enzymatically active dimer and reversible hybridization of the second seed sequence to the second nucleotide sequence of the first enzymatically active dimer; and
wherein the first nucleotide and the second nucleotide are configured to dimerize to form the first enzymatically active dimer upon reversible hybridization of the first nucleotide sequence to the first seed sequence and reversible hybridization of the second nucleotide sequence to the second seed sequence.

6. The system of any of claims 1-5, wherein one or more of the nucleic acid target, the first nucleotide, and the second nucleotide is a DNA molecule.

7. The system of claim 6, wherein the first nucleotide is a DNA molecule, wherein the second nucleotide is a DNA molecule,
wherein the first nucleotide comprises a first thymine base,
wherein the second nucleotide comprises a second thymine base,
wherein the first thymine base and the second thymine base are configured to be brought into proximity by the reversible hybridization of the first nucleotide sequence to the first target sequence and the reversible hybridization of the second nucleotide sequence to the second target sequence of any of claims 1-3, and
wherein the first thymine base and the second thymine base are configured to dimerize when exposed to ultraviolet light.

8. The system of claim 2, wherein one or more of the first probe and the second probe is a DNA molecule.

9. The system of claim 8, wherein the first probe is a DNA molecule,
wherein the second probe is a DNA molecule,
wherein the first probe comprises a first thymine base,
wherein the second probe comprises a second thymine base,
wherein the first thymine base and the second thymine base are configured to be brought into proximity by the reversible hybridization of the first nucleotide sequence to the first probe sequence and the reversible hybridization of the second nucleotide sequence to the third probe sequence of claim 2, and
wherein the first thymine base and the second thymine base are configured to dimerize when exposed to ultraviolet light.

10. The system of claim 1, wherein one or more of the first reporter and the second reporter is a DNA molecule.

11. The system of claim 10, wherein the first reporter is a DNA molecule, wherein the second reporter is a DNA molecule,
wherein the first reporter comprises a first thymine base,
wherein the second reporter comprises a second thymine base,
wherein the first thymine base and the second thymine base are configured to be brought into proximity by the reversible hybridization of the first reporter sequence to the first nucleotide sequence and the reversible hybridization of the second reporter sequence to the second nucleotide sequence of claim 1, and
wherein the first thymine base and the second thymine base are configured to dimerize when exposed to ultraviolet light.

12. The system of claim 2, wherein the reporter is a DNA molecule.

13. The system of claim 4, wherein one or more of the third nucleotide and the fourth nucleotide is a DNA molecule.

14. The system of claim 13, wherein the third nucleotide is a DNA molecule, wherein the fourth nucleotide is a DNA molecule,
wherein the third nucleotide comprises a first thymine base,
wherein the fourth nucleotide comprises a second thymine base,
wherein the first thymine base and the second thymine base are configured to be brought into proximity by the reversible hybridization of the third nucleotide sequence to the first nucleotide sequence and the reversible hybridization of the fourth nucleotide sequence to the second nucleotide sequence of claim 4, and
wherein the first thymine base and the second thymine base are configured to dimerize when exposed to ultraviolet light.

15. The system of claim 5, wherein one or more of the first seed nucleotide and the second seed nucleotide is a DNA molecule.

16. The system of claim 15, wherein the first seed nucleotide is a DNA molecule, wherein the second seed nucleotide is a DNA molecule,
wherein the first seed nucleotide comprises a first thymine base,
wherein the second seed nucleotide comprises a second thymine base,
wherein the first thymine base and the second thymine base are configured to be brought into proximity by the reversible hybridization of the first seed nucleotide sequence to the first nucleotide sequence and the reversible hybridization of the second seed nucleotide sequence to the second nucleotide sequence of claim 5, and
wherein the first thymine base and the second thymine base are configured to dimerize when exposed to ultraviolet light.

17. The system of any of claims 1-5, wherein one or more of the nucleic acid target, the first nucleotide, and the second nucleotide is an RNA molecule.

18. The system of claim 2, wherein one or more of the first probe and the second probe is an RNA molecule.

19. The system of claim 1, wherein one or more of the first reporter and the second reporter is an RNA molecule.

20. The system of claim 2, wherein the reporter is an RNA molecule.

21. The system of claim 4, wherein one or more of the third nucleotide and the fourth nucleotide is an RNA molecule.

22. The system of claim 5, wherein one or more of the first seed nucleotide and the second seed nucleotide is an RNA molecule.

23. The system of claim 3, wherein the first enzymatic sequence and the second enzymatic sequence are configured to form a deoxyribozyme or a ribozyme.

24. The system of claim 4, wherein the third enzymatic sequence and the fourth enzymatic sequence are configured form a deoxyribozyme or a ribozyme.

25. The system of any of claims 1-24, wherein one or more of the first nucleotide and the second nucleotide comprises one or more abasic sites configured to decrease the energy associated with dissociating the first nucleotide or the second nucleotide and a hybridization partner.

26. The system of any of claims 1-25, wherein the first nucleotide sequence comprises one or more mismatch bases compared to the first target sequence configured to decrease the energy associated with dissociating the first nucleotide sequence and the first target sequence, and/or
wherein the second nucleotide sequence comprises one or more mismatch bases compared to the second target sequence configured to decrease the energy associated with dissociating the first nucleotide sequence and the first target sequence.

27. The system of any of claims 1, 2, 8-12, and 18-20, wherein the first reporter sequence comprises one or more mismatch bases compared to the second reporter sequence configured to decrease the energy associated with dissociating the first reporter sequence and the second reporter sequence.

28. The system of any of claims 2, 8, 12, 18, and 20, wherein the first probe sequence comprises one or more mismatch bases compared to the first nucleotide sequence configured to decrease the energy associated with dissociating the first probe sequence and the first nucleotide sequence,
wherein the second probe sequence comprises one or more mismatch bases compared to the first reporter sequence configured to decrease the energy associated with dissociating the second probe sequence and the first reporter sequence,
wherein the third probe sequence comprises one or more mismatch bases compared to the second nucleotide sequence configured to decrease the energy associated with dissociating the third probe sequence and the second nucleotide sequence, and/or wherein the fourth probe sequence comprises one or more mismatch bases compared to the second reporter sequence configured to decrease the energy associated with dissociating the fourth probe sequence and the second nucleotide sequence.

29. The system of any of claims 4, 13, 21, and 24 wherein the first nucleotide sequence comprises one or more mismatch bases compared to the third nucleotide sequence configured to decrease the energy associated with dissociating the first nucleotide sequence and the third nucleotide sequence, and/or
wherein the second nucleotide sequence comprises one or more mismatch bases compared to the fourth nucleotide sequence configured to decrease the energy associated with dissociating the second nucleotide sequence and the fourth nucleotide sequence.

30. The system of any of claims 5, 15, 16, and 22 wherein the first nucleotide sequence comprises one or more mismatch bases compared to the first seed sequence configured to decrease the energy associated with dissociating the first nucleotide sequence and the first seed sequence, and/or
wherein the second nucleotide sequence comprises one or more mismatch bases compared to the second seed sequence configured to decrease the energy associated with dissociating the second nucleotide sequence and the second seed sequence.

31. The system of any of claims 1, 2, 8-12, 18-20, and 28 wherein the first reporter moiety in proximity with the second reporter moiety is configured to increase fluorescence at a predetermined wavelength.

32. The system of any of claims 1, 2, 8-12, 18-20, 27, and 28 wherein the first reporter moiety and the second reporter moiety are configured for Förster resonance energy transfer.

33. The system of any of claims 1, 2, 8-12, 18-20, 27, and 28 wherein the first reporter moiety in proximity with the second reporter moiety is configured to decrease fluorescence at a predetermined wavelength.

34. The system of any of claims 1, 2, 8-12, 18-20, 27, and 28 wherein the first reporter moiety is a fluorophore, and wherein the second reporter moiety is a quencher.

35. The system of any of claims 3-5, 13-16, 21-24, 29, and 30, wherein the first enzymatic sequence and the second enzymatic sequence are configured to form a peroxidase-mimicking G-quadruplex deoxyribozyme.

36. The system of any of claims 4, 13, 14, 21, 24, and 29, wherein the third enzymatic sequence and the fourth enzymatic sequence are configured to form a peroxidase-mimicking G-quadruplex deoxyribozyme.

37. The system of any of claims 35 and 36, wherein the one or more first substrates comprises 2,2′-azino-bis(3-ethylbenzothiozoline-6-sulfonic acid) (ABTS) or 3,3′,5,5′-tetramethylbenzidine (TMB), wherein the system further comprises hydrogen peroxide (H₂O₂), and wherein the system further comprises hemin.

38. The system of any of the claims 1-37, wherein a second target comprises a third target sequence and a fourth target sequence, and wherein the system further comprises:
a fifth nucleotide comprising a fifth nucleotide sequence configured to reversibly hybridize the third target sequence;
a sixth nucleotide comprising a sixth nucleotide sequence configured to reversibly hybridize the fourth target sequence;
wherein the fifth nucleotide and the sixth nucleotide are configured to dimerize to form a second dimer upon reversible hybridization of the fifth nucleotide sequence to the third target sequence and reversible hybridization of the sixth nucleotide sequence to the fourth target sequence;
a third reporter comprising a third reporter moiety and a third reporter sequence coupled to the third reporter moiety, wherein the third reporter sequence is configured to reversibly hybridize the fifth nucleotide sequence; and
a fourth reporter comprising a fourth reporter moiety and a fourth reporter sequence coupled to the fourth reporter moiety, wherein the fourth reporter sequence is configured to reversibly hybridize the sixth nucleotide sequence, and wherein the fourth reporter sequence is configured to reversibly hybridize the third reporter sequence;
wherein the third reporter and the fourth reporter are configured to dimerize to form a second reporter dimer upon reversible hybridization of the third reporter sequence to the fifth nucleotide sequence of the second dimer and reversible hybridization of the fourth reporter sequence to the sixth nucleotide sequence of the second dimer,
wherein the third reporter moiety is configured to produce a second reporter moiety signal, wherein the fourth reporter moiety is configured to alter the second reporter moiety signal when the third reporter moiety and the fourth reporter moiety are in proximity,
wherein reversible hybridization of the third reporter sequence to the fourth reporter sequence is configured to bring the third reporter moiety and the fourth reporter moiety into proximity, and
wherein reversible hybridization of one or more of the third reporter sequence to the fifth nucleotide sequence of the second dimer and the fourth reporter sequence to the sixth nucleotide sequence of the second dimer is configured to bring the third reporter moiety and the fourth reporter moiety out of proximity.

39. The system of any of the claims 1-37, wherein a second target comprises a third target sequence and a fourth target sequence, and wherein the system further comprises:
a fifth nucleotide comprising a fifth nucleotide sequence configured to reversibly hybridize the third target sequence;
a sixth nucleotide comprising a sixth nucleotide sequence configured to reversibly hybridize the fourth target sequence;
wherein the fifth nucleotide and the sixth nucleotide are configured to dimerize to form a second dimer upon reversible hybridization of the fifth nucleotide sequence to the third target sequence and reversible hybridization of the sixth nucleotide sequence to the fourth target sequence;
a third probe comprising a fifth probe sequence and a sixth probe sequence, wherein the fifth probe sequence is configured to reversibly hybridize the fifth nucleotide sequence;
a fourth probe comprising a seventh probe sequence and an eighth probe sequence, wherein the seventh probe sequence is configured to reversibly hybridize the sixth nucleotide sequence;
wherein the third probe and the fourth probe are configured to dimerize to form a second probe dimer upon reversible hybridization of the fifth probe sequence to the fifth nucleotide sequence of the second dimer and reversible hybridization of the seventh probe sequence to the sixth nucleotide sequence of the second dimer; and
a second reporter comprising:
a third reporter sequence configured to reversibly hybridize the sixth probe sequence,
a fourth reporter sequence coupled to the third reporter sequence, wherein the fourth reporter sequence is configured to reversibly hybridize the eighth probe sequence, and wherein the fourth reporter sequence is configured to reversibly hybridize the third reporter sequence,
a third reporter moiety coupled to third reporter sequence, and
a fourth reporter moiety coupled to the fourth reporter sequence;
wherein the third reporter moiety is configured to produce a second reporter moiety signal, wherein the fourth reporter moiety is configured to alter the second reporter moiety signal when the third reporter moiety and the fourth reporter moiety are in proximity,
wherein reversible hybridization of the third reporter sequence to the fourth reporter sequence is configured to bring the third reporter moiety and fourth reporter moiety into proximity, and wherein reversible hybridization of one or more of the third reporter sequence to the sixth probe sequence of the second probe dimer and the fourth reporter sequence to the eighth probe sequence of the second probe dimer is configured to bring the third reporter moiety and the fourth reporter moiety out of proximity.

40. The system of any of the claims 1-37, wherein a second target comprises a third target sequence and a fourth target sequence, and wherein the system further comprises:
a fifth nucleotide comprising a fifth nucleotide sequence and a third enzymatic sequence coupled to the fifth nucleotide sequence, wherein the fifth nucleotide sequence is configured to reversibly hybridize the third target sequence;
a sixth nucleotide comprising a sixth nucleotide sequence and a fourth enzymatic sequence coupled to the sixth nucleotide sequence, wherein the sixth nucleotide sequence is configured to reversibly hybridize the fourth target sequence;
wherein the fifth nucleotide and the sixth nucleotide are configured to dimerize to form a third enzymatically active dimer upon reversible hybridization of the fifth nucleotide sequence to the third target sequence and reversible hybridization of the sixth nucleotide sequence to the fourth target sequence; and
one or more second substrates;
wherein the third enzymatically active dimer is configured to convert the one or more second substrates into one or more second products, and
wherein the one or more second products are different from the one or more first products.

41. The system of claim 40, further comprising:
a seventh nucleotide comprising a seventh nucleotide sequence and a fifth enzymatic sequence coupled to the seventh nucleotide sequence, wherein the seventh nucleotide sequence is configured to reversibly hybridize the fifth nucleotide sequence; and
an eighth nucleotide comprising an eighth nucleotide sequence and a sixth enzymatic sequence coupled to the eighth nucleotide sequence, wherein the eighth nucleotide sequence is configured to reversibly hybridize the sixth nucleotide sequence;
wherein the seventh nucleotide and the eighth nucleotide are configured to dimerize to form a fourth enzymatically active dimer upon reversible hybridization of the seventh nucleotide sequence to the fifth nucleotide sequence of the third enzymatically active dimer and the eighth nucleotide sequence to the sixth nucleotide sequence of the third enzymatically active dimer, and
wherein the fourth enzymatically active dimer is configured to convert the one or more second substrates into one or more second products.

42. The system of any of claims 40 or 41, further comprising:
a third seed nucleotide comprising a third seed sequence configured to reversibly hybridize the fifth nucleotide sequence; and
a fourth seed nucleotide comprising a fourth seed sequence configured to reversibly hybridize the sixth nucleotide sequence;
wherein the third seed nucleotide and the fourth seed nucleotide are configured to dimerize to form a second seed dimer upon reversible hybridization of the third seed nucleotide to the fifth nucleotide sequence of the third enzymatically active dimer and reversible hybridization of the fourth seed sequence to the sixth nucleotide sequence of the third enzymatically active dimer; and
wherein the fifth nucleotide and the sixth nucleotide are configured to dimerize to form the third enzymatically active dimer upon reversible hybridization of the fifth nucleotide sequence to the third seed sequence and reversible hybridization of the sixth nucleotide sequence to the fourth seed sequence.

43. The system of any of claims 40-42, wherein the one or more first substrates is 2,2′-azino-bis(3-ethylbenzothiozoline-6-sulfonic acid) (ABTS), wherein the one or more first products is an ABTS radical, wherein the one or more second substrates is 3,3′,5,5′-tetramethylbenzidine (TMB), and wherein the one or more second products is a TMB radical.

44. The system of any of claims 38-43, wherein the nucleic acid target comprises the second target.

45. The system of any of claims 1-43, further comprising a module comprising:
a sensor configured to detect ultraviolet light;
a pathway configured to output a comparison comparing the cumulative ultraviolet light detected by the sensor to a predetermined threshold; and
a display configured to display a value associated with one or more of the ultraviolet light detected by the sensor, the cumulative ultraviolet light detected by the sensor, and the comparison.

46. The system of any of claims 1-43, further comprising an ultraviolet light source.

47. A method of detecting a nucleic acid target comprising a first target sequence and a second target sequence, wherein the method comprises:
providing a first nucleotide comprising a first nucleotide sequence;
reversibly hybridizing the first nucleotide sequence to the first target sequence;
providing a second nucleotide comprising a second nucleotide sequence;
reversibly hybridizing the second nucleotide sequence to the second target sequence;
dimerizing the first nucleotide and the second nucleotide to form a first dimer upon reversibly hybridizing the first nucleotide sequence to the first target sequence and reversibly hybridizing the second nucleotide sequence to the second target sequence;
dissociating the first dimer and the nucleic acid target;
providing a reporter complex comprising:
a first reporter comprising a first reporter moiety and a first reporter sequence coupled to the first reporter moiety, wherein the first reporter moiety is configured to produce a first reporter moiety signal, and
a second reporter comprising a second reporter moiety and a second reporter sequence coupled to the second reporter moiety, wherein the second reporter sequence is reversibly hybridized to the first reporter sequence, wherein the second reporter moiety is configured to alter the first reporter moiety signal when the first reporter moiety and the second reporter moiety are in proximity, and wherein reversible hybridization of the first reporter sequence to the second reporter sequence is configured to bring the first reporter moiety and the second reporter moiety into proximity;
dissociating the first reporter sequence and the second reporter sequence;
reversibly hybridizing the first reporter sequence to the first nucleotide sequence;
reversibly hybridizing the second reporter sequence to the second nucleotide sequence;
bringing the first reporter moiety and the second reporter moiety out of proximity by reversibly hybridizing the first reporter sequence to the first nucleotide sequence of the first dimer and/or reversibly hybridizing the second reporter sequence to the second nucleotide sequence of the first dimer, and
detecting a change in the first reporter moiety signal.

48. The method of claim 47, further comprising dimerizing the first reporter and the second reporter to form a reporter dimer upon reversibly hybridizing the first reporter sequence to the first nucleotide sequence of the first dimer and reversibly hybridizing the second reporter sequence to the second nucleotide sequence of the first dimer.

49. The method of claim 48, further comprising dissociating the first dimer and the first reporter dimer.

50. A method of detecting a nucleic acid target comprising a first target sequence and a second target sequence, wherein the method comprises:
providing a first nucleotide comprising a first nucleotide sequence;
reversibly hybridizing the first nucleotide sequence to the first target sequence;
providing a second nucleotide comprising a second nucleotide sequence;
reversibly hybridizing the second nucleotide sequence to the second target sequence;
dimerizing the first nucleotide and the second nucleotide to form a first dimer upon reversibly hybridizing the first nucleotide sequence to the first target sequence and reversibly hybridizing the second nucleotide sequence to the second target sequence;
dissociating the first dimer and the nucleic acid target;
providing a first probe comprising a first probe sequence and a second probe sequence;
reversibly hybridizing the first probe sequence to the first nucleotide sequence;
providing a second probe comprising a third probe sequence and a fourth probe sequence;
reversibly hybridizing the third probe sequence to the second nucleotide sequence;
provide a reporter comprising:
a first reporter moiety configured to produce a first reporter moiety signal,
a first reporter sequence coupled to the first reporter moiety, wherein the first reporter sequence is configured to reversibly hybridize the second probe sequence,
a second reporter moiety configured to alter the first reporter moiety signal when the first reporter moiety and the second reporter moiety are in proximity, and
a second reporter sequence, wherein the second reporter sequence is coupled to the second reporter moiety, wherein the second reporter sequence is coupled to the first reporter sequence, wherein the second reporter sequence is reversibly hybridized to the first reporter sequence, wherein the second reporter sequence is configured to reversibly hybridize the second nucleotide sequence, and wherein reversible hybridization of the first reporter sequence to the second reporter sequence is configured to bring the first reporter moiety and the second reporter moiety into proximity; and
dissociating the first reporter sequence and the second reporter sequence;
reversibly hybridizing the first reporter sequence to the second probe sequence;
reversibly hybridizing the second reporter sequence to the fourth probe sequence;
bringing the first reporter moiety and the second reporter moiety out of proximity by reversible hybridizing the first reporter sequence to the second probe sequence and/or reversible hybridizing the second reporter sequence to the fourth probe sequence, and detecting a change in the first reporter moiety signal.

51. The method of claim 50, further comprising dimerizing the first probe and the second probe to form a first probe dimer upon reversible hybridizing the first probe sequence to the first nucleotide sequence of the first dimer and reversibly hybridizing the third probe sequence to the second nucleotide sequence of the first dimer.

52. A method of detecting a nucleic acid target comprising a first target sequence and a second target sequence, wherein the method comprises:
providing a first nucleotide comprising a first nucleotide sequence and a first enzymatic sequence coupled to the first nucleotide sequence;
reversibly hybridizing the first nucleotide sequence to the first target sequence;
providing a second nucleotide comprising a second nucleotide sequence and a second enzymatic sequence coupled to the second nucleotide sequence;
reversibly hybridizing the second nucleotide sequence to the second target sequence;
dimerizing the first nucleotide and the second nucleotide to form a first enzymatically active dimer upon reversibly hybridizing the first nucleotide sequence to the first target sequence and reversibly hybridizing the second nucleotide sequence to the second target sequence, wherein the first enzymatically active dimer is configured to convert one or more first substrates into one or more first products;
providing the one or more first substrates;
converting the one or more first substrates to the one or more first products; and
detecting one or more of a decrease in amount of the one or more first substrates, a decrease in the concentration of the one or more first substrates, an increase in amount of the one or more first products, and an increase in concentration of the one or more first products.

53. The method of claim 52, further comprising:
dissociating the first enzymatically active dimer and the nucleic acid target;
providing a first seed nucleotide comprising a first seed sequence;
reversibly hybridizing the first seed sequence the first nucleotide sequence;
providing a second seed nucleotide comprising a second seed sequence;
reversibly hybridizing the second seed nucleotide to the second nucleotide sequence; and
dimerizing the first seed nucleotide and the second seed nucleotide to form a first seed dimer upon reversibly hybridizing the first seed nucleotide to the first nucleotide sequence of the first enzymatically active dimer and reversibly hybridizing the second seed sequence to the second nucleotide sequence of the first enzymatically active dimer.

54. The method of claim 52, further comprising:
dissociating the first enzymatically active dimer and the nucleic acid target;
providing a third nucleotide comprising a third nucleotide sequence and a third enzymatic sequence coupled to the third nucleotide sequence;
reversibly hybridizing the third nucleotide sequence to the first nucleotide sequence;
providing a fourth nucleotide comprising a fourth nucleotide sequence and a fourth enzymatic sequence coupled to the fourth nucleotide sequence;
reversibly hybridizing the fourth nucleotide sequence to the second nucleotide sequence; and
dimerizing the third nucleotide and the fourth nucleotide to form a second enzymatically active dimer upon reversibly hybridizing the third nucleotide sequence to the first nucleotide sequence of the first enzymatically active dimer and reversibly hybridizing the fourth nucleotide sequence to the second nucleotide sequence of the first enzymatically active dimer, wherein the second enzymatically active dimer is configured to convert the one or more first substrates into the one or more first products.

55. The method of any of claims 47-54, wherein the first nucleotide comprises a first nucleotide thymine base;
wherein the second nucleotide comprises a second nucleotide thymine base; and
wherein the step of dimerizing the first nucleotide and the second nucleotide comprises:
bringing the first nucleotide thymine base into proximity with the second nucleotide thymine base,
providing an ultraviolet light source, and
forming one or more bonds coupling the first nucleotide thymine base and the second nucleotide thymine base.

56. The method of any of claims 47-49, wherein the first reporter comprises a first reporter thymine base;
wherein the second reporter comprises a second reporter thymine base; and
wherein the step of dimerizing the first reporter and the second reporter comprises:
bringing the first reporter thymine base into proximity with the second reporter thymine base,
providing an ultraviolet light source, and
forming one or more bonds coupling the first reporter thymine base and the second reporter thymine base.

57. The method of claim 54, wherein the third nucleotide comprises a third nucleotide thymine base;
wherein the fourth nucleotide comprises a fourth nucleotide thymine base; and
wherein the step of dimerizing the third nucleotide and the fourth nucleotide comprises:
bringing the third nucleotide thymine base into proximity with the fourth nucleotide thymine base,
providing an ultraviolet light source, and
forming one or more bonds coupling the third nucleotide thymine base and the fourth nucleotide thymine base.

58. The method of claim 53, wherein the first seed nucleotide comprises a first seed thymine base;
wherein the second seed nucleotide comprises a second seed nucleotide thymine base; and
wherein the step of dimerizing the first seed nucleotide and the second seed nucleotide comprises:
bringing the first seed nucleotide thymine base into proximity with the second seed nucleotide thymine base,
providing an ultraviolet light source, and
forming one or more bonds coupling the first seed nucleotide thymine base and the second seed nucleotide thymine base.

59. The method of any of claims 47-51 and 56, wherein the step of detecting the change in a signal produced by one or more of the first reporter moiety and the second reporter moiety comprises detecting one or more of an increase in fluorescence at a first predetermined wavelength and a decrease in fluorescence at a second predetermined wavelength.

60. The method of claim 59, wherein the increase in fluorescence at the first predetermined wavelength is due to Förster resonance energy transfer, or wherein the decrease in fluorescence at the second wavelength is due to Förster resonance energy transfer.

61. The method of claim 59, wherein the first reporter moiety is a fluorophore, wherein the second reporter moiety is a quencher, and
wherein the decrease in fluorescence at the second predetermined wavelength is due to quenching a fluorescent signal emitted by the first reporter moiety.

62. The method of claim 59, wherein the first reporter moiety is a fluorophore, wherein the second reporter moiety is a quencher, and
wherein the increase in fluorescence at the first predetermined wavelength is due to de-quenching a fluorescent signal emitted by the first reporter moiety.

63. The method of any of claims 52-54, 57, and 58, wherein the step of detecting one or more of the decrease in amount of the one or more first substrates, the decrease in the concentration of the one or more first substrates, the increase in amount of one or more first products, and the increase in concentration of one or more first products comprises detecting the increase in the amount of a 2,2′-azino-bis(3-ethylbenzothiozoline-6-sulfonic acid) (ABTS) radical, detecting the increase in the concentration of an ABTS radical, detecting the increase in the amount of a 3,3′,5,5′-tetramethylbenzidine (TMB) radical, or detecting the increase in the concentration of a TMB radical.

64. The method of any of the claims 47-63, wherein a second target comprises a third target sequence and a fourth target sequence, the method further comprising:
providing a fifth nucleotide comprising a fifth nucleotide sequence;
reversibly hybridizing the fifth nucleotide sequence to the third target sequence;
providing a sixth nucleotide comprising a sixth nucleotide sequence;
reversibly hybridizing the sixth nucleotide sequence to the fourth target sequence;
dimerizing the fifth nucleotide and the sixth nucleotide to form a second dimer, upon reversibly hybridizing the fifth nucleotide sequence to the third target sequence and reversibly hybridizing the sixth nucleotide sequence to the fourth target sequence;
dissociating the second dimer and the target;
providing a second reporter complex comprising:
a third reporter comprising a third reporter moiety and a third reporter sequence coupled to the third reporter moiety, and wherein the third reporter moiety is configured to produce a second reporter moiety signal, and
a fourth reporter comprising a fourth reporter moiety and a fourth reporter sequence coupled to the fourth reporter moiety, wherein the fourth reporter sequence is reversibly hybridized to the third reporter sequence, wherein the fourth reporter moiety is configured to alter the second reporter moiety signal when the third reporter moiety and the fourth reporter moiety are in proximity, and wherein reversible hybridization of the third reporter sequence to the fourth reporter sequence is configured to bring the third reporter moiety and the fourth reporter moiety into proximity;
dissociating the third reporter sequence and the fourth reporter sequence;
reversibly hybridizing the third reporter sequence to the fifth nucleotide sequence;
reversibly hybridizing the fourth reporter sequence to the sixth nucleotide sequence;
bringing the third reporter moiety and the fourth reporter moiety out of proximity by reversibly hybridizing the third reporter sequence to the fifth nucleotide sequence of the second dimer and/or reversibly hybridizing the fourth reporter sequence to the sixth nucleotide sequence of the second dimer, and
detecting a change in the second reporter moiety signal.

65. The method of claim 64, further comprising dimerizing the third reporter and the fourth reporter to form a second reporter dimer upon reversibly hybridizing the third reporter sequence to the fifth nucleotide sequence and reversibly hybridizing the fourth reporter sequence to the sixth nucleotide sequence.

66. The method of claim 65, further comprising dissociating the second dimer and the second reporter dimer.

67. The method of any of the claims 47-63, wherein a second target comprises a third target sequence and a fourth target sequence, the method further comprising:
providing a fifth nucleotide comprising a fifth nucleotide sequence;
reversibly hybridizing the fifth nucleotide sequence to the third target sequence;
providing a sixth nucleotide comprising a sixth nucleotide sequence;
reversibly hybridizing the sixth nucleotide sequence to the fourth target sequence;
dimerizing the fifth nucleotide and sixth nucleotide to form a second dimer upon reversibly hybridizing the fifth nucleotide sequence to the third target sequence and reversibly hybridizing the sixth nucleotide sequence to the fourth target sequence;
dissociating the second dimer and the second target;
providing a third probe comprising a fifth probe sequence and a sixth probe sequence;
reversibly hybridizing the fifth probe sequence to the fifth nucleotide sequence;
providing a fourth probe comprising a seventh probe sequence and an eighth probe sequence;
reversibly hybridizing the seventh probe sequence to the sixth nucleotide sequence;
providing a second reporter comprising:
a third reporter moiety configured to produce a second reporter moiety signal,
a third reporter sequence coupled to the third reporter moiety, wherein the third reporter sequence is configured to reversibly hybridize the sixth probe sequence,
a fourth reporter moiety configured to alter the second reporter moiety signal when the third reporter moiety and the fourth reporter moiety are in proximity, and
a fourth reporter sequence, wherein the fourth reporter sequence is coupled to the fourth reporter moiety, wherein the fourth reporter sequence is coupled to the third reporter sequence, wherein the fourth reporter sequence is reversibly hybridized to the third reporter sequence, wherein the fourth reporter sequence is configured to reversibly hybridize the eighth probe sequence, and wherein reversible hybridization of the third reporter sequence to the fourth reporter sequence is configured to bring the third reporter moiety and the fourth reporter moiety into proximity, and
dissociating the third reporter sequence and the fourth reporter sequence;
reversibly hybridizing the third reporter sequence to the sixth probe sequence;
reversibly hybridizing the fourth reporter sequence to the eighth probe sequence;
bringing the third reporter moiety and the fourth reporter moiety out of proximity by reversibly hybridizing the third reporter sequence to the sixth probe sequence and/or reversibly hybridizing the fourth reporter sequence to the eighth probe sequence, and detecting a change in the second reporter moiety signal.

68. The method of claim 67, further comprising dimerizing the third probe and the fourth probe to form a second probe dimer upon reversibly hybridizing the fifth probe sequence to the fifth nucleotide sequence and reversibly hybridizing the seventh probe sequence to the sixth nucleotide sequence.

69. The method of any of the claims 47-63, wherein a second target comprises a third target sequence and a fourth target sequence, the method further comprising:
providing a fifth nucleotide comprising a fifth nucleotide sequence and a third enzymatic sequence coupled to the fifth nucleotide sequence;
reversibly hybridizing the fifth nucleotide sequence to the third target sequence;
providing a sixth nucleotide comprising a sixth nucleotide sequence and a fourth enzymatic sequence coupled to the sixth nucleotide sequence;
reversibly hybridizing the sixth nucleotide sequence to the fourth target sequence;
dimerizing the fifth nucleotide and the sixth nucleotide to form a third enzymatically active dimer upon reversibly hybridizing the fifth nucleotide sequence to the third target sequence and reversibly hybridizing the sixth nucleotide sequence to the fourth target sequence, wherein the third enzymatically active dimer is configured to convert one or more second substrates into one or more second products, wherein the one or more second products are different from the one or more first products;
providing the one or more second substrates;
converting the one or more second substrates into the one or more second products; and
detecting one or more of a decrease in amount of the one or more second substrates, a decrease in the concentration of the one or more second substrates, an increase in amount of the one or more second products, and an increase in concentration of the one or more second products.

70. The method of claim 69, further comprising:
dissociating the third enzymatically active dimer and the second target;
providing a third seed nucleotide comprising a third seed sequence;
reversibly hybridizing the third seed sequence to the fifth nucleotide sequence;
providing a fourth seed nucleotide comprising a fourth seed sequence;
reversibly hybridizing the fourth seed sequence to the sixth nucleotide sequence; and
dimerizing the third seed nucleotide and the fourth seed nucleotide to form a second seed dimer upon reversibly hybridizing the third seed sequence to the fifth nucleotide sequence of the third enzymatically active dimer and reversibly hybridizing the fourth seed sequence to the sixth nucleotide of the third enzymatically active dimer.

71. The method of claim 69, further comprising:
dissociating the third enzymatically active dimer and the second target;
providing a seventh nucleotide comprising a seventh nucleotide sequence and a fifth enzymatic sequence coupled to the seventh nucleotide sequence;
reversibly hybridizing the seventh nucleotide sequence to the fifth nucleotide sequence;
providing an eighth nucleotide comprising an eighth nucleotide sequence and a sixth enzymatic sequence coupled to the eighth nucleotide sequence;
reversibly hybridizing the eighth nucleotide sequence to the sixth nucleotide sequence; and
dimerizing the seventh nucleotide and the eighth nucleotide to form a fourth enzymatically active dimer upon reversibly hybridizing the seventh nucleotide sequence to the fifth nucleotide sequence of the third enzymatically active dimer and reversibly hybridizing the eighth nucleotide sequence to the sixth nucleotide sequence of the third enzymatically active dimer, wherein the fourth enzymatically active dimer is configured to convert the one or more second substrates into the one or more second products.

72. The method of any of claims 69-71, wherein the one or more first substrates is 2,2′-azino-bis(3-ethylbenzothiozoline-6-sulfonic acid) (ABTS), wherein the one or more first products is an ABTS radical, wherein the one or more second substrates is 3,3′,5,5′-tetramethylbenzidine (TMB), and wherein the one or more second products is a TMB radical.

73. The method of any of claims 64-72, wherein the nucleic acid target comprises the second target.

74. The method of any of claims 47-73, further comprising determining one or more of the concentration of one or more cations, anions, and salts and the amount of one or more cations, anions, and salts of a composition comprising the nucleic acid target.
